(19) United States
(12) Patent Application Publication
(10) Pub. No.: US 2013/0129681 A1
(43) Pub. Date: May 23, 2013

(54) METHODS FOR DIAGNOSING SOLID TUMORS

(71) Applicant: NODALITY, INC., South San Francisco, CA (US)

(72) Inventors: Todd COVEY, San Carlos, CA (US); Alessandra Cesano, Redwood City, CA (US); Michael Gulrajani, Daly City, CA (US)

(73) Assignee: NODALITY, INC., South San Francisco, CA (US)

(21) Appl. No.: 13/645,325

(22) Filed: Oct. 4, 2012

Related U.S. Application Data
(60) Provisional application No. 61/542,910, filed on Oct. 4, 2011.

(51) Int. Cl. G01N 33/68

(52) U.S. Cl. 424/85.7; 435/7.1; 435/7.23

(57) ABSTRACT

An embodiment of the present invention is useful for identifying tumor cells in bladder cancer washes, pleural effusions, biliary tumor cells and circulating tumor cells in whole blood. Subsequent analysis may identify therapeutic treatments based on a single cell analysis of activatable elements in cell signaling pathways. This analysis can be useful for diagnosis, prognosis, therapy selection and monitoring of solid tumor diseases.

### CROSS-REFERENCE

This application claims the benefit of U.S. Provisional Application No. 61/542,910, filed Oct. 4, 2011, which application is incorporated herein by reference.

### BACKGROUND OF THE INVENTION

Despite great gains in knowledge over the past several decades in the fields of genetics and cellular and molecular biology, this expansion of knowledge has not translated into commensurate advances in the diagnosis or prognosis of disease, or the ability to predict or assess response to therapy. New methods for diagnosis and prognosis that harness the advances in the biologic sciences are needed.

### SUMMARY OF THE INVENTION

One embodiment of the present invention is a process for determining the signaling capability of cancer cells in an effort to aid in diagnosis, prognosis, selecting therapy, or monitoring disease. In another embodiment of the present invention is a process for determining the signaling capability of bladder cancer cells in an effort to aid in diagnosis, prognosis, selecting therapy, or monitoring disease. In one embodiment the process comprises providing cells suspected of being bladder cancer cells, from an individual, identifying and selecting single cells that have an epithelial cell marker, determining aneuploidy of the cells, detecting and excluding cells that are undergoing apoptosis, contacting the cells with EGF, determining the level of activatable elements in the single cells, comprising p-AKT and p-ERK; and contacting the cells with a PI3kinase or MAP kinase pathway inhibitor. One embodiment of the process further comprises administering a therapeutic agent to the individual based on the determination of the activatable elements of the single cells. One embodiment of the process uses reagents designed to detect EpCAM, Cytokeratin, CD45, estrogen receptor, HER2, CD44, vimentin, cadherin, or EGFR and determines aneuploidy using the DNA stain, DAPI. Another embodiment of the process determines if a cell is undergoing apoptosis by measuring c-PARP, cleaved cytokeratin 18, cleaved caspase, cleaved caspase 3, cytochrome C, apoptosis inducing factor, MCL-1, BCL-2, BCL-XL, PUMA, NOXA, Bim, Bad, Bad, Bax, p53, c-myc proto-oncogene, APO-1/Fas/CD95, Annexin-V, 7-AAD, Amine Aqua, trypan blue, or propidium iodide. The process can detect less than 9 cells per 10,000,000 cells by using a flow cytometer or mass spectrometer. The invention also provides for a process to analyze solid tumor cells circulating in whole blood and biliary tumor cells using the methods described above. Another embodiment is a process for analyzing solid tumor cells circulating in whole blood, comprising obtaining whole blood containing circulating tumor cells (CTCs), selecting individual CTCs that are epithelial cells, determining if the CTC is malignant, excluding any CTC that is undergoing apoptosis, and detecting activatable elements in the MAP Kinase or PI3 kinase signaling pathways by contacting the cells with EGF and detecting activatable elements in the MAP kinase or PI3 kinase signaling pathways.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows Signaling in HT-1376 Bladder Cancer (BC) Cell Line.

FIG. 2 shows Example of a BC sample that is functionally similar to BC cell line.

FIG. 3 compares pAKT and pERK in patient and cell line samples for MCSF-4

FIG. 4 compares pAKT and pERK in patient and cell line samples for MCSF-2.

FIG. 5 compares pAKT and pERK in patient and cell line sample for MCSF-6.

FIG. 6 compares pAKT and pERK in patient and cell line samples for MCSF-15.

### DETAILED DESCRIPTION OF THE INVENTION

The present methods incorporate information disclosed in other applications and texts. The following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

Also, patents and applications that are incorporated by reference include U.S. Pat. Nos. 7,381,535, 7,393,656, 7,563,584, 7,695,924, 7,695,926, 7,939,278, 8,148,094, 8,187,885, 8,198,037, 8,206,939, 8,214,157, 8,227,202, 8,242,248; U.S. Ser. Nos. 11/338,957, 11/655,789, 12/061,565, 12/125,759, 12/125,763, 12/229,476, 12/432,239, 12/432,720, 12/471,158, 12/501,274, 12/501,295, 12/538,643, 12/551,333, 12/581,536, 12/606,869, 12/617,438, 12/687,873, 12/688,851, 12/703,741, 12/713,165, 12/730,170, 12/778,847, 12/784,478, 12/877,998, 12/910,769, 13/082,306, 13/091,971, 13/094,731, 13/094,735, 13/094,737, 13/098,902, 13/098,923, 13/098,932, 13/098,939, 13/384,181; International Applications Nos. PCT/US2011/001565, PCT/US2011/065675, PCT/US2011/026117, PCT/US2011/029845, PCT/US2011/048332; and U.S. Ser. Nos. 60/304,434, 60/310,141, 60/646,757, 60/787,908, 60/957,160, 61/048,657, 61/048,886, 61/048,920, 61/055,362, 61/079,537, 61/079,551, 61/079,579, 61/079,766, 61/085,789, 61/087,555, 61/104,666, 61/106,462, 61/108,803, 61/113,823, 61/120,320, 61/144,68, 61/144,955, 61/146,276, 61/151,387, 61/153,627, 61/155,373, 61/156,754, 61/157,900, 61/162,598, 61/162,673, 61/170,348, 61/176,420, 61/177,935, 61/181,211, 61/182,518, 61/182,638, 61/186,619, 61/216,825, 61/218,718, 61/226,878, 61/236,281, 61/240,193, 61/240,613, 61/241,773, 61/245,000, 61/254,131, 61/263,281, 61/265,585, 61/265,743, 61/306,665, 61/306,872, 61/307,829, 61/317,187, 61/327,347, 61/350,864, 61/353,155, 61/373,199, 61/374,613, 61/381,067, 61/382,793, 61/423,918, 61/436,534, 61/440,523, 61/469,812, 61/499,127, 61/515,660, 61/521,221, 61/542,910, 61/557,831, 61/558,343, 61/565,391, 61/565,929, 61/565,935, 61/591,122, 61/640,794, 61/658,092, 61/664,426, and 61/693,429.

Some commercial reagents, protocols, software and instruments that are useful in some embodiments of the present invention are available from Becton Dickinson, see their Website <<www.bdbiosciences.com/features/products/>>, and Beckman Coulter, see their website, <<www.beckmancoulter.com/Default.asp?bhfv=7>>. Relevant articles include High-content single-cell drug screening with phosphospecific flow cytometry, Krutzik et al., Nature Chemical Biology, 23 Dec. 2007; Irish et al., FLt3 ligand Y591 duplication and Bcl-2 over expression are detected in acute myeloid leukemia cells with high levels of phosphorylated wild-type p53, Neoplasia, 2007; Irish et al. Mapping normal and cancer cell signaling networks: towards single-cell proteomics, Nature, Vol. 6 146-155, 2006; Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol (2007), 78:8 8.17.1-20; Krutzik, P. O., et al., Coordinate analysis of murine immune cell surface markers and intracellular phosphoproteins by flow cytometry, J. Immunol. 2005 Aug. 15; 175(4):2357-65; Krutzik, P. O., et al., Characterization of the murine immunological signaling network with phosphospecific flow cytometry, J Immunol. 2005 Aug. 15; 175(4):2366-73; Shulz et al., Current Protocols in Immunology 2007, 78:8.17.1-20; Stelzer et al., Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 October; 55(2):61-70; Hanahan D., Weinberg, “The Hallmarks of Cancer”, Cell (2000) January 7; 100(1) 57-70; and Krutzik et al., “High content single cell drug screening with phophosphospecific flow cytometry”, Nat. Chem. Biol. 2008 February; 4(2):132-42. Experimental and process protocols and other helpful information can be found at <<http:/proteomics.stanford.edu>>. The articles and other references cited below are also incorporated by reference in their entireties for all purposes. More specific procedures can be found in the following manuscripts: Rosen D B, Putta S, Covey T et al.,” Distinct Patterns of DNA Damage Response and Apoptosis Correlate with Jak/Stat and PI3Kinase Response Profiles in Human Acute Myelogenous Leukemia” 2010. PLoS ONE. 5 (8): e12405; Kornblau S M, Minden M D, Rosen D B, Putta S, Cohen A, Covey T, et al., “Dynamic Single-Cell Network Profiles in Acute Myelogenous Leukemia Are Associated with Patient Response to Standard Induction Therapy” 2010. Clinical Cancer Research. 16 (14): 3721-33 January 31; Rosen D B et al., “Functional Characterization of FLT3 Receptor Signaling Deregulation in AML by Single Cell Network Profiling (SCNP)” 2010. PLoS ONE. 5 (10): e13543. Covey T M, Putta S, Cesano A.“Single cell network profiling (SCNP): mapping drug and target interactions. Assay Drug Dev Technol” (2010);8:321-43.

The term “patient” or “individual” as used herein includes humans as well as other mammals. The methods generally involve determining the status of an activatable element. The methods also involve determining the status of a plurality of activatable elements.

The analysis of a cell and the determination of the status of an activatable element can comprise classifying a cell as a cell that is correlated to a patient response to a treatment. In some embodiments, the patient response is selected from the group consisting of complete response, partial response, nodular partial response, no response, progressive disease, stable disease and adverse reaction.

The classification of a rare cell according to the status of an activatable element can comprise classifying the cell as a cell that can be correlated with minimal residual disease or emerging resistance (see U.S. Ser. No. 61/048,886).

The classification of a cell according to the status of an activatable element can comprise selecting a method of treatment. Examples of treatment methods include, but are not limited to, compounds that control some of the symptoms, such as aspirin and antihistamines, compounds that stimulate red blood cell production, such as erythropoietin or darbepoietin, compounds that reduce platelet production, such as hydroxyurea, anagrelide, and interferon-alpha, compounds that increase white blood cell production, such as G-CSF, chemotherapy, biological therapy, radiation therapy, phlebotomy, blood cell transfusion, bone marrow transplantation, peripheral stem cell transplantation, umbilical cord blood transplantation, autologous stem cell transplantation, allogeneic stem cell transplantation, syngeneic stem cell transplantation, surgery, induction therapy, maintenance therapy, and other therapy.

### I. Overview

In one embodiment, the present method can identify and analyze cells that have originated in solid tumors. In another embodiment, the present method can identify and analyze cells that are later transported into other parts of the body (for example, circulating tumor cells or CTCs). In another embodiment, the cells are analyzed in whole blood. In another embodiment of the present invention, cells are analyzed to determine whether they may be bladder cancer. One embodiment of the present invention allows a researcher to look at a heterogeneous population of cells and focus on a particular subtype and thereafter characterize that subtype in ways described below. Reference will be made to bladder cancer, but it will be used as an example of one type of solid tumor. Other tumors will also be included in the present methods.

In one embodiment, cells are isolated from body samples and tested. The tests can determine, as non-limiting examples; cell type, such as distinguishing epithelial cells from leukocytes; whether cells are cancerous, or have aneuploidy; cell health/apoptosis state; and their signaling state. In one embodiment, the method of the present invention can test samples having a heterogeneous cell population and determine if there are cancer cells present in the population, their signaling state and whether they are likely to apoptose.

In one embodiment, cells are isolated from body samples, such as, but not limited to, smears, sputum, biopsies, secretions, cerebrospinal fluid, bile, sera, whole blood, ascites, plasma, lavage or rinse of cavities, such as a bladder wash, lymph fluid, urine and feces, or tissue which has been removed from organs, such as breast, lung, intestine, skin, cervix, prostate, and stomach. The biological specimen can be a fraction of the above specimen or a derivative of the specimen. For example, a tissue sample can comprise a region of functionally related cells or adjacent cells. Such samples can comprise complex populations of cells, which can be assayed as a population, or separated into sub-populations. In one embodiment, the cells are analyzed in whole blood. In another embodiment, cellular samples can be separated by centrifugation, elutriation, density gradient separation, apheresis, affinity selection, panning, FACS, centrifugation with Hypaque, etc. By using antibodies specific for markers identified with particular cell types, a relatively homogeneous population of cells may be obtained. Alternatively, a heterogeneous cell population can be used. Cells can also be separated by using filters. For example, whole blood can be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. Ser. No. 09/790,673. Once a sample is obtained, it can be used directly, cryopreserved, or maintained in appropriate culture medium for short periods of time. Methods to isolate one or more cells for use according to the methods of this invention are performed according to standard techniques and protocols well-established in the art.

One embodiment of the invention is a process whereby the level of detection of rare cells, which can be circulating tumor cells, is less than 1 cell per 100,000, 2 cells per 100,000, 3 cells per 100,000, 4 cells per 100,000, 5 cells per 100,000, 6 cells per 100,000, 7 cells per 100,000, 8 cells per 100,000, or 9 cells per 100,000. One embodiment of the invention is a process whereby the level of detection is less than 1 cell per 1,000,000, 2 cells per 1,000,000, 3 cells per 1,000,000, 4 cells per 1,000,000, 5 cells per 1,000,000, 6 cells per 1,000,000, 7 cells per 1,000,000, 8 cells per 1,000,000, or 9 cells per 1,000,000. Another embodiment of the invention can detect cells of interest at a level below 1, 2, 3, 4, 5, 6, 7, 8, 9 cells per 10,000,000 (see U.S. Ser. No. 12/432,720).

The origin of the tumor or neoplastic cells can be from solid tumors. If the original is a solid tumor, then the solid tumor may be any solid tumor amenable to sampling for direct or indirect analysis. Solid tumors include but are not limited to head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma.

In some embodiments, the invention provides a method of classifying a cell by determining the presence or absence of an increase or decrease in activation level of one or more activatable elements in the cell upon treatment with one or more modulators, and classifying the cell based on the presence or absence of the increase or decrease in the activation of the activatable element. In some embodiments of the invention, the activation level of the activatable element is determined by contacting the cell with one or more binding elements that are specific for an activation state of the activatable element. One method is termed single cell network profiling (SCNP).

SCNP can involve the use of various reagents for determining the status of activatable elements as well as other cell states. In one embodiment of the invention, a panel of reagents is used to analyze the cell of interest.

In one embodiment, SCNP offers several methods to improve the decision making and treatment of bladder cancer. In patients with advanced disease, it would provide the basis for addition of targeted therapy to existing systemic options. In patients undergoing cystectomy, pathway signatures would correlate with risk of future recurrent/metastatic disease and, therefore, serve as a tool to identify patients who will most benefit from adjuvant chemotherapy. In patients with non-muscle invasive disease, analysis of pathway activation could identify patients with those tumors that have acquired the means for future progression. Identification of very high risk patients, with a low-grade tumor stage would allow for early cystectomy when the disease is potentially curable.

In another embodiment of the invention, a researcher could detect CTCs in whole blood. The detection of CTCs has considerable utility in the clinical management of patients with solid cancers. However, the phenotypic heterogeneity of CTCs and their low numbers in the bloodstream of patients means that it is difficult to isolate these cells. In patients with metastatic breast cancer, CTC counts greater than five CTCs per 7.5 ml of blood before the start of systemic therapy were associated with a shorter median progression-free survival and overall survival (Cristofanilli et al., 2004, 2008; Botteri et al., 2010).

In one embodiment, the cells are analyzed in whole blood so that the cells are in the relevant physiological environment at testing. This environment may be important during a pharmacodynamic assay as the cells are maintained in contact with the relevant drug (after dosing) up to the time of analysis. Otherwise, the cells may not display the drug effect during testing.

Additionally, methods are provided for adjusting a cell signaling analysis based on the quality of cells in the sample. Unhealthy cells can reduce the quality of a cell sample. An unhealthy cell may not have the capacity to signal, or signal in response to a modulator, e.g., an external stimulus, at the same level as a healthy cell. For example, an unhealthy cell may not have the ability to activate an activatable element to the same level as a healthy cell. In one embodiment, a method is provided herein for reducing the signal to noise ratio in a single cell analysis (see PCT App. No. WO/2012/024546).

A. Single Cell Network Profiling (SCNP) Assay

Single cell network profiling (SCNP) is a method that can be used to analyze activatable elements such as phosphorylation sites of proteins in signaling pathways in single cells in response to modulation by signaling agonists or inhibitors (e.g., kinase inhibitors). Other examples of activatable elements include an acetylation site, a ubiquitination site, a methylation site, a hydroxylation site, a SUMOylation site, or a cleavage site. Activation of an activatable element can involve a change in cellular localization or conformation state of individual proteins, or change in ion levels, oxidation state, pH, etc. It is useful to classify cells and to provide diagnosis or prognosis as well as other activities, such as drug screening or research, based on the cell classifications. SCNP is one method that can be used in conjunction with an analysis of cell health, but there are other methods that may benefit from this analysis. Embodiments of SCNP are shown in references cited herein. See for example, U.S. Pat. No. 7,695,924.

In one embodiment, SCNP can be used to generate a cell signaling profile. In another embodiment, SCNP can be used to measure apoptosis in cells stained with an antibody with specific affinity to cleaved PARP (c-PARP), for example, after the cells have been exposed to one or more modulators, such as chemotherapy drugs or other treatments. Other cell health markers may be quantified as well. In one embodiment, the one or more cell health markers can be MCL-1 and/or c-PARP. Other markers include caspase cleavage products such as dye substrates, c-PARP, cleaved cytokeratin 18, cleaved caspase, cleaved caspase 3, cytochrome C, apoptosis inducing factor (AIF), Inhibitor of Apoptosis (IAP) family members, as well as other molecules such as Bcl-2 family members including anti-apoptotic proteins (MCL-1, BCL-2, BCL-XL), BH3-only apoptotic sensitizers (PUMA, NOXA, Bim, Bad), and pro-apoptotic proteins (Bad, Bax) (see below), p53, c-myc proto-oncogene, APO-1/Fas/CD95, growth stimulating genes, or tumor suppressor genes, mitochondrial membrane dyes, Annexin-V, 7-AAD, Amine Aqua, trypan blue, propidium iodide or other viability dyes.

A significant fraction of cells with high cleaved PARP (c-PARP) levels or low MCL-1 levels, before or without treatment with, e.g., a modulator, can indicate that some cells are undergoing apoptosis before treatment with a modulator. For some experiments, the activation state or activation level of an activatable element in an untreated sample of cells may be attributable to cells undergoing apoptosis due to one or more reasons related to sample processing (e.g., shipment conditions, cryogenic storage, thawing of cryogenically stored cells, etc.). If the apoptotic cells are not physically removed from the analysis, or data from apoptotic cells is not removed from an analysis of cell signaling data, apoptotic cells (which can be c-PARP positive or MCL-1 negative) can negatively impact the measurement of treatment (e.g., with a modulator) induced activation of an activatable element, e.g., phosphorylation of a phosphorylation site, and cause a misleading view of the signaling potential for the specific cell population being studied.

The level of apoptosis (and correlated background activation of an activatable element, e.g., phosphorylation noise) in thawed cell populations can be attributable to sample handling, including a freeze-thaw process. Therefore, in one embodiment, the use of an antibody directed to a cell health marker, e.g., an activatable element, e.g., c-PARP or MCL-1, in combination with phospho-specific and cell lineage specific antibodies allows subsequent analysis and exclusion (or non-inclusion) from an analysis of a cell signaling profile (e.g., activation level of an activatable element) of cells that are unhealthy. Incorporating an apoptosis analysis using an MCL-1 or anti-c-PARP antibody (anti-c-PARP), for example, into an assay, e.g., an assay for the activation state or activation level of an activatable element, can improve the quality and consistency of data, e.g., cell signaling data, within and across cell populations in, e.g., a SCNP assay and other assays. More generally, an apoptosis assay with an anti-c-PARP or MCL-1 antibody, for example, can be used to measure freeze-thaw quality as a quality-control test. In one embodiment apoptosis can be measured and quantified in samples that have been frozen and thawed and cells undergoing apoptosis after thawing can be excluded from an experiment before the cells are stimulated with a modulator. Other markers of apoptosis, e.g., Annexin V, can be employed to direct which cells can be physically excluded. Annexin V is an extracellular apoptosis marker.

In one embodiment, a method is provided comprising the following steps: a) thawing a sample of cells; b) contacting said sample of cells with Aqua reagent (or other amino reactive dye) to distinguish dead (nonviable cells) from live cells in said sample of cells; c) contacting said sample of cells with an experimental treatment (e.g., a modulator) to modulate phospho-proteins (e.g., signaling agonist or inhibitor can be used as a modulator); d) fixing and permeabilizing cells in said sample of cells; e) staining said sample of cells with a fluorophore conjugated antibody cocktail comprising an antibody (e.g., and antibody directed to c-PARP or MCL-1) plus a phospho-specific antibody plus a cell lineage specific antibody; f) analyzing said sample of cells with SCNP technology; g) using anti-c-PARP antibody (for example) parameter to identify and analyze cells that are not undergoing apoptosis; and h) analyzing signaling pathway protein phosphorylation levels in c-PARP “negative” cells that appear not to be apoptosing or “healthy.”

In one embodiment Annexin V is also used in combination with a antibody that binds or recognizes a cell health marker (see e.g., Koopman G, Reutelingsperger C P, Kuijten G A M et al. “Annexin V for flow cytometric detection of phosphatidylserine expression on B cells undergoing apoptosis” Blood (1994) 84 (5): 1415-20 for information on Annexin V) Annexin V stain (available, e.g., from Sigma-Aldrich and eBioscience, San Diego, Calif.) can be used for early apoptosis detection for intact cells. Live cells can be stained with Annexin V which binds phosphatidylserine, which is normally intracellular, but is shifted to the cell surface when cells are undergoing apoptosis or other forms of cell death. PARP cleavage can be a late apoptosis readout, and fixed and permeabilized cells can be used to analyze PARP cleavage. PARP cleavage can result from caspase activation because PARP is a caspase target. To assay for cleaved PARP, cells can be fixed and permeabilized and then stained for c-PARP. In one embodiment, a method is provided comprising staining for Annexin V on live cells using a Paraformaldehyde (PFA)/methanol insensitive dye; washing away excess Annexin V stain; fixing and permeabilizing cells (with PFA/methanol); and using intracellular staining for one or more intracellular cell health markers of interest, including c-PARP or MCL-1, an earlier apoptosis marker.

In one embodiment, a method is provided to use Annexin V conjugated beads or columns or other device to isolate and physically bind and remove apoptotic cells. This embodiment involves incubating a sample with Annexin-V coated purifying element (such as magnetic beads) and running the sample through a magnetic column to select for healthy cells, that is cells that are negative for Annexin-V expression (Annexin-V^{neg}), also known as negative selection. Cells could then be used in SCNP assays or used as desired for other readouts.

B. Tumor Cell Analysis

The development of metastases in a number of solid tumor cancers is associated with poor survival. Significantly improved approaches for determination of recurrence risk, earlier detection of metastatic disease, and responsiveness to treatment are needed. Circulating tumor cells (CTC) continue to elicit significant interest as a marker for these three, and potentially other uses. For example, CTC are isolated in all breast cancer stages and elevated CTC pretreatment or after one cycle of treatment, is an adverse prognostic factor. However, the absolute number of CTCs may not be the most relevant biomarker since it is believed that only some CTC are capable of establishing metastatic niches. These CTC may have more invasive and metastatic properties with increased mesenchymal and stem cell marker expression. The FDA approved positive selection technology for CTC (CellSearch) while successful in identifying CTC in a number of types of solid tumor cancers, has several limitations including poor CTC yields relative to the original sample prior to enrichment; and CTC with low epithelial marker (EpCAM) expression, such as those from basal and normal-like breast cancer subtypes, may be missed. These CTC, if they could be detected, may be of clinical relevance as the most aggressive breast cell lines and tumors, such as basal subtype, have low, or no, epithelial marker expression and increased mesenchymal and stem cell-like marker expression. These mesenchymal markers have been associated with increased aggressiveness and metastatic potential.

In one embodiment, bladder cancer cells are analyzed. Bladder cancer is any of several types of malignant growths of the urinary bladder. It is a disease in which abnormal cells multiply without control in the bladder. The bladder is a hollow, muscular organ that stores urine; it is located in the pelvis. The most common type of bladder cancer begins in cells lining the inside of the bladder and is called transitional cell carcinoma (sometimes urothelial cell carcinoma).

The gold standard for diagnosing bladder cancer is biopsy obtained during cystoscopy. Sometimes it is an incidental finding during cystoscopy. Urine cytology can be obtained in voided urine or at the time of the cystoscopy (“bladder washing”). Cytology is very specific (a positive result is highly indicative of bladder cancer) but suffers from low sensitivity (inability of a negative result to reliably exclude bladder cancer). There are newer non-invasive urine bound markers available as aids in the diagnosis of bladder cancer, including human complement factor H-related protein, high-molecular-weight carcinoembryonic antigen, and nuclear matrix protein 22 (NMP22).

The diagnosing of bladder cancer can also be done with a Cysview™ guided fluorescence cystoscopy, as an adjunct to conventional white-light cystoscopy. This procedure improves the detection of bladder cancer and reduces the rate of early tumour recurrence, compared with white-light cystoscopy alone. Cysview cystoscopy detects more cancer and reduce recurrency.

The treatment of bladder cancer depends on how deep the tumor invades into the bladder wall. Superficial tumors (those not entering the muscle layer) can be “shaved off” using an electrocautery device attached to a cystoscope. Immunotherapy in the form of BCG instillation is also used to treat and prevent the recurrence of superficial tumors.

Untreated, superficial tumors may gradually begin to infiltrate the muscular wall of the bladder. Tumors that infiltrate the bladder require more radical surgery where part or all of the bladder is removed (a cystectomy) and the urinary stream is diverted. In some cases, skilled surgeons can create a substitute bladder (a neobladder) from a segment of intestinal tissue, but this largely depends upon patient preference, age of patient, renal function, and the site of the disease.

A combination of radiation and chemotherapy can also be used to treat invasive disease. It has not yet been determined how the effectiveness of this form of treatment compares to that of radical ablative surgery.

There are approximately 71,000 new cases of bladder cancer (BC) in the U.S. and 360,000 worldwide (Jemal, A. et al. CA Cancer J Clin 59:2009). It is one of the most expensive of all cancers to treat due to the length of treatment ($98,000-$187,000 (Dx to Death) (Botteman, M. F. et al. Pharmacoeconomics 21:2003). Transitional cell carcinoma constitutes 90-95% of bladder, ureter and renal pelvis cancers. Squamous cell carcinoma can be up to 7% and Adenocarcinoma is rare, constituting less than 3%.

Bladder cancer is classified in stages; Tis, Ta, and T1 are superficial and T2, T3A, T3B, and T4 are invasive. Treatment of superficial lesions, such as T0, T1S, T1 and low grade T2, is by endoscopic resection with cystoscopy, repeated every three months. Transurethral resection (TUR) of the bladder is a surgical procedure that is used both to diagnose BC and to remove cancerous tissue. During TUR surgery, a cytoscope is passed into the bladder through the urethra. A tool called a resectoscope is used to remove the cancer for biopsy and to burn away any remaining cancer cells. Partial cystectomy can be used for patients whose tumors are not amenable to transureteral resection.

The prognosis for superficial tumors is that about 20% of patients are cured by removal of tumor, 50-70% recur one or more times, and 10-30% progress to invasive and potentially lethal disease. For muscle infiltrating tumors, 50% will develop metastatic disease. Overall, patients undergoing radical cystectomy for muscle invasive disease have approximately 50% 5 year survival.

Bladder cancer chemotherapy remains disappointing because of the toxicity, which reinforces the rationale for targeted therapy. Bladder cancer is strongly associated with aberrations of signaling pathways involved with proliferation, angiogenesis, and apoptosis. Additionally, Epithelial Growth Factor Receptor (EGFR) signaling is involved in cell cycle progression, inhibition of apoptosis, tumor cell motility, invasion and metastasis. A typical treatment regime uses MVAC, which includes methotrexate, vinblastine, doxorubicin, and cisplatin. Gemcitabine and cisplatin have been shown to be equivalent while being less toxic. Neoadjuvant and adjuvant therapy can be used to improve survival, especially in the metastatic setting.

Epidermal growth factor receptor (EGFR) is known to be overexpressed in bladder cancer. High level expression is associated with higher tumor grade and stage and with decreased recurrence-free interval (Kassouf et al. J of Uro (2008). Also, EGFR protein expression level is not generally considered as a reliable biomarker of anti-EGFR inhibitors activity.

In one embodiment of the invention, cells are screened for tumorgenicity. The cells can be populations including solid tumor cells. In another embodiment heterogeneous population of cells are analyzed using the SCNP method using a panel of reagents that can determine, among other things, cell type, such as distinguishing epithelial cells from leukocytes for example, whether cells are cancerous, aneuploidy, cell health/apoptosis state, and their signaling state. One preferred embodiment screen cell populations for bladder cancer. In one embodiment of the invention, various reagents are employed in this analysis. For example, reagents, such as binding agents may come from the following groups to determine the following categories of interest: the phenotype of a cell of interest, DNA content, apoptosis, and intracellular signaling. In one embodiment, the binding agents include the following: Phenotyping, including: Cytokeratin, EpCAM, CD45; DNA content, including: DAPI or other DNA content markers; Apoptosis, including: c-PARP; and intracellular signaling including: pERK, pAKT. The above panel is designed to provide reagents to identify epithelial cells and distinguish them from leukocytes. It also contains a DNA stain, DAPI to detect DNA content in cells and identify cell with aneuploidy status, such as cancer cells. An apoptosis marker such as c-PARP is used to determine the health of a cell. Signaling markers are used to determine the pathways that are activated in the cell. This particular panel can be employed for bladder cancer. Other panels can be used for other cancers or solid tumors. More information is provided below for each of the phenotyping markers.

In one embodiment, a cell sample is provided and analyzed as follows. A blood sample is taken and cells having epithelial cell markers are identified. For example, in one embodiment, a flow cytometer is used to identify cells with light scattering properties of whole, fully intact cells with nuclei (indicated by DAPI staining). The cells should have a nucleus and a CD45 expression low than lymphoid or myeloid cells. The cells should have high expression of cytokeratin and EpCAM.

In one embodiment of the invention, the method identifies malignant epithelial cells by looking at increased DNA content and aneuploidy (DNA Index 2.61 “Epithelial G1 “DAPI MFI/Lymphoid G1 DAPI MFI). Thereafter, the SCNP process is used to measure signaling and apoptosis (c-PARP). Basal signaling is measured using pAKT and pERK (a 2-3 fold response can be seen upon EGF modulation). Evoked signaling to measure function is determined with EGF stimulation using a PI3kinase inhibitor, for example, GDC-0941 bismesylate and pAKT and pERK are measured. One embodiment of the present invention also measures activatable elements such as PI3kinase, mTOR, MEK, RAF, RAS, SOS, GRB2, or STATs. Drug responses can be analyzed by modulating the cells in the presence of a candidate drug, such as a PI3kinase inhibitor (GDC-0941) other drugs known to modulate EGF can be used. In another aspect of the invention, other drugs can be employed with the method such as those identified in U.S. Ser. Nos. 12/687,873 and 12/703,741.

In other embodiments, it should be understood that other methods may be used to select epithelial cells than the use of the markers listed above. The same is also true for the other members of the panel. Substitutions may be made for DAPI to detect a cancer or a cell with aneuploidy, for c-PARP to detect the health of a cell, and pAKT or pErk for cell signaling.

In one embodiment, reagents are used to identify cells having extracellular phenotyping markers which indicate that a cell is an epithelial cell. In one embodiment, those markers include, but are not limited to, cytokeratin, CD45, EpCAM, estrogen receptor, HER2, CD44, Vimentin, EGFR, and N-Cahedrin.

In one embodiment, the presence or absence of these markers indicate a cell of interest, such as an epithelial cell. In one embodiment, the cells will be missing or have little of one or more of the following markers: CD45 or the proteins known to bind directly or indirectly to CD45. In one embodiment, the cells will have more of one or more of the following markers: EpCAM, cytokeratin, cahedrins, vimentin, estrogen receptor, HER2, CD44, and EGFR. One or more of each of the above markers may be employed to select cells of interest.

Cytokeratins are proteins of keratin-containing intermediate filaments found in the intracytoplasmic cytoskeleton of epithelial tissue. The term “cytokeratin” began to be used in the late 1970s (for example, see “Intermediate-sized filaments of human endothelial cells” by Franke, Schmid, Osborn and Weber, Franke W W, Schmid E, Osborn M, Weber K (1979). “Intermediate-sized filaments of human endothelial cells”. J. Cell Biol. 81 (3): 570-580) when the protein subunits of keratin intermediate filaments inside cells were first being identified and characterized. In 2006 a new systematic nomenclature for keratins was created and now the proteins previously called “cytokeratins” are simply called keratins, Schweizer J, Bowden P E, Coulombe P A, Langbein L, Lane E B, Magin T M, Maltais L, Omary M B, Parry D A, Rogers M A, Wright M W (2006). “New consensus nomenclature for mammalian keratins”. J. Cell Biol. 174 (2): 169-174. Over 25,000 published articles exist in the biomedical research literature that used the term “cytokeratin”.

There are two types of cytokeratins: the acidic type I cytokeratins and the basic or neutral type II cytokeratins. Cytokeratins are usually found in pairs comprising a type I cytokeratin and a type II cytokeratin. Basic or neutral cytokeratins include CK1, CK2, CK3, CK4, CK5, CK6, CK7, CK8 and CK9. Acidic cytokeratins are CK10, CK12, CK13, CK14, CK16, CK17, CK18, CK19 and CK20. The cytokeratins cannot be divided into low versus high molecular weight solely based on their charge. Expression of these cytokeratins is frequently organ or tissue specific. As an example, CK7 is typically expressed in the ductal epithelium of the genitourinary (GU) tract and CK20 most commonly in the gastrointestinal (G1) tract, CURRENT Diagnosis & Treatment: Obstetrics & Gynecology 2011. Chapter 52. Premalignant & Malignant Disorders of the Ovaries & Oviducts. Kathleen M. Brennan, MD, Vicki V. Baker, MD, & Oliver Dorigo, MD. Histopathologists employ such distinctions to detect the cell of origin of various tumors. The methods used by the histopathologist can be used in whole are in part with various aspects of the present invention.

The subsets of cytokeratins in which an epithelial cell expresses depends largely on the type of epithelium, the moment in the course of terminal differentiation and the stage of development. Thus this specific cytokeratin fingerprint allows the classification of all epithelia upon their cytokeratin expression profile. Furthermore this applies also to the malignant counterparts of the epithelia (carcinomas), as the cytokeratin profile tends to remain constant when an epithelium undergoes malignant transformation. The main clinical implication is that the study of the cytokeratin profile by immunohistochemistry techniques is a tool of immense value widely used for tumor diagnosis and characterization in surgical pathology, Walid M S, Osborne T J, Robinson J S (2009). “Primary brain sarcoma or metastatic carcinoma?” Indian J Cancer 46 (2): 174-175. The methods used tumor diagnosis and characterization can be used in whole are in part with various aspects of the present invention.

CD45 antigen, also known as the Protein tyrosine phosphatase receptor type C (PTPRC) is an enzyme that, in humans, is encoded by the PTPRC gene, Tchilian E Z, Beverley P C (2002). “CD45 in memory and disease.” Arch. Immunol. Ther. Exp. (Warsz.) 50 (2): 85-93. PTPRC, also known as CD45 antigen (CD stands for cluster of differentiation), which was originally called leukocyte common antigen, Ishikawa H, Tsuyama N, Abroun S, et al. (2004). “Interleukin-6, CD45 and the src-kinases in myeloma cell proliferation.” Leuk. Lymphoma 44 (9): 1477-81.

The protein encoded by this gene is a member of the protein tyrosine phosphatase (PTP) family. PTPs are known to be signaling molecules that regulate a variety of cellular processes including cell growth, differentiation, mitotic cycle, and oncogenic transformation. This PTP contains an extracellular domain, a single transmembrane segment and two tandem intracytoplasmic catalytic domains, and thus belongs to receptor type PTP. This gene is specifically expressed in hematopoietic cells. This PTP has been shown to be an essential regulator of T-cell and B-cell antigen receptor signaling. It functions through either direct interaction with components of the antigen receptor complexes or by activating various Src family kinases required for the antigen receptor signaling. This PTP also suppresses JAK kinases, and, thus, functions as a negative regulator of cytokine receptor signaling. Four alternatively spliced transcripts variants of this gene, which encode distinct isoforms, have been reported.

It is a type I transmembrane protein that is in various forms present on all differentiated hematopoietic cells except erythrocytes and plasma cells that assists in the activation of those cells (a form of co-stimulation). It is expressed in lymphomas, B-cell chronic lymphocytic leukemia, hairy cell leukemia, and acute nonlymphocytic leukemia. A monoclonal antibody to CD45 is used routinely clinical immunohistochemistry laboratories to differentiate between histological sections from lymphomas and carcinomas, Leong, Anthony S-Y; Cooper, Kumarason; Leong, F Joel W-M (2003). Manual of Diagnostic Cytology (2 ed.). Greenwich Medical Media, Ltd. pp. 121-124.

EpCAM is epithelial cell adhesion molecule that in humans is encoded by the EPCAM gene, Linnenbach A J, Seng B A, Wu S, Robbins S, Scollon M, Pyrc J J, Druck T, Huebner K (April 1993). “Retroposition in a family of carcinoma-associated antigen genes”. Mol Cell Biol 13 (3): 1507-15; and Calabrese G, Crescenzi C, Morizio E, Palka G, Guerra E, Alberti S (April 2001). “Assignment of TACSTD1 (alias TROP1, M4S1) to human chromosome 2p21 and refinement of mapping of TACSTD2 (alias TROP2, M1S1) to human chromosome 1p32 by in situ hybridization”. Cytogenet Cell Genet 92 (1-2): 164-5. EpCAM has also been designated as TACSTD1 (tumor-associated calcium signal transducer 1) and CD326 (cluster of differentiation 326).

EpCAM is a pan-epithelial differentiation antigen that is expressed on almost all carcinomas. Its constitutional function is being elucidated. It is intricately linked with the Cadherin-Catenin pathway and hence the fundamental WNT pathway responsible for intracellular signaling and polarity. It has been used as an immunotherapeutic target in the treatment of gastrointestinal, urological and other carcinomas, Chaudry M A, Sales K, Ruf P, Lindhofer H, Winslet M C (April 2007). “EpCAM an immunotherapeutic target for gastrointestinal malignancy: current experience and future challenges”. Br. J. Cancer 96 (7): 1013-9. It is expressed in undifferentiated pluripotent stem cells. Sundberg, M; Jansson, L; Ketolainen, J; Pihlajamaki, H; Suuronen, R; Skottman, H; Inzunza, J; Hovatta, O et al. (2009). “CD marker expression profiles of human embryonic stem cells and their neural derivatives, determined using flow-cytometric analysis, reveal a novel CD marker for exclusion of pluripotent stem cells.” Stem Cell Research 2 (2): 113-24.

EpCAM is a carcinoma-associated antigen and is a member of a family that includes at least two type I membrane proteins. This antigen is expressed on most normal epithelial cells and gastrointestinal carcinomas and functions as a homotypic calcium-independent cell adhesion molecule. The antigen is being used as a target for immunotherapy treatment of human carcinomas.

Edrecolomab, catumaxomab and other monoclonal antibodies are designed to bind to it, Punt C J, Nagy A, Douillard J Y, Figer A, Skovsgaard T, Monson J, Barone C, Fountzilas G, Riess H, Moylan E, Jones D, Dethling J, Colman J, Coward L, MacGregor S (August 2002). “Edrecolomab alone or in combination with fluorouracil and folinic acid in the adjuvant treatment of stage III colon cancer: a randomised study” Lancet 360 (9334): 671-7.

The epidermal growth factor receptor (EGFR; ErbB-1; HER1 in humans) is the cell-surface receptor for members of the epidermal growth factor family (EGF-family) of extracellular protein ligands. See Herbst R S (2004). “Review of epidermal growth factor receptor biology”. Int. J. Radiat. Oncol. Biol. Phys. 59 (2 Suppl): 21-6. The epidermal growth factor receptor is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Mutations affecting EGFR expression or activity could result in cancer. See Zhang H, Berezov A, Wang Q, Zhang G, Drebin J, Murali R, Greene M I “ErbB receptors: from oncogenes to targeted cancer therapies” (August 2007). J. Clin. Invest. 117 (8): 2051-8. Epidermal Growth Factor was discovered by Stanley Cohen of Vanderbilt University along with Rita Levi-Montalcini for which both received the Nobel prize in Physiology or Medicine in 1986.

EGFR (epidermal growth factor receptor) exists on the cell surface and is activated by binding of its specific ligands, including epidermal growth factor and transforming growth factor α (TGFα) among others. ErbB2 has no known direct activating ligand, and may be in an activated state constitutively or become active upon heterodimerization with other family members such as EGFR. Upon activation by its growth factor ligands, EGFR undergoes a transition from an inactive monomeric form to an active homodimer—although there is some evidence that preformed inactive dimers may also exist before ligand binding. In addition to forming homodimers after ligand binding, EGFR may pair with another member of the ErbB receptor family, such as ErbB2/Her2/neu, to create an activated heterodimer. There is also evidence to suggest that clusters of activated EGFRs form, although it remains unclear whether this clustering is important for activation itself or occurs subsequent to activation of individual dimers.

EGFR dimerization stimulates its intrinsic intracellular protein-tyrosine kinase activity. As a result, autophosphorylation of several tyrosine (Y) residues in the C-terminal domain of EGFR occurs. These include Y992, Y1045, Y1068, Y1148 and Y1173 as shown in the diagram to the left. See Downward J, Parker P, Waterfield M D (1984). “Autophosphorylation sites on the epidermal growth factor receptor” Nature 311 (5985): 483-5. This autophosphorylation elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and JNK pathways, leading to DNA synthesis and cell proliferation. See Oda K, Matsuoka Y, Funahashi A, Kitano H (2005). “A comprehensive pathway map of epidermal growth factor receptor signaling”. Mol. Syst. Biol. 1 (1): 2005.0010. Such proteins can modulate cell phenotypes and states such as inducing changes in migration, adhesion, and proliferation. Activation of the receptor is important for the innate immune response in human skin. See Sorensen, O. E., Tapa, D. R., Roupé, K. M., et al.“Injury-induced innate immune response in human skin mediated by transactivation of the epidermal growth factor receptor”. (2006) J Clin Invest. 116 (7): 1878-1885. The kinase domain of EGFR can also cross-phosphorylate tyrosine residues of other receptors it is aggregated with, and can itself be activated in that manner.

Mutations that lead to overexpression of EGFR (also known as upregulation or overactivity) have been associated with a number of cancers, including lung cancer, anal cancers (see Walker, F.; Abramowitz, L.; Benabderrahmane, D.; Duval, X.; Descatoire, V. R.; Hénin, D.; Lehy, T. R. S.; Aparicio, T. “Growth factor receptor expression in anal squamous lesions: modifications associated with oncogenic human papillomavirus and human immunodeficiency virus” (2009) Human Pathology 40 (11): 1517-1527 and glioblastoma multiforme. In this latter case a more or less specific mutation of EGFR, called EGFRvIII is often observed. See Kuan, C. T., Wikstrand, C. J., Bigner, D. D. “EGF mutant receptor vIII as a molecular target in cancer therapy” Endocr. Relat. Cancer (2001), June 8 (2): 83-96. Mutations, amplifications or misregulations of EGFR or family members are implicated in about 30% of all epithelial cancers.

Mutations involving EGFR could lead to its constant activation, which could result in uncontrolled cell division—a predisposition for cancer. See Lynch T J, Bell D W, Sordella R, Gurubhagavatula S, Okimoto R A, Brannigan B W, Harris P L, Haserlat S M, Supko J G, Haluska F G, Louis D N, Christiani D C, Settleman J, Haber D A (May 2004) “Activating mutations in the epidermal growth factor receptor underlying responsiveness of non-small-cell lung cancer to gefitinib”. N. Engl. J. Med. 350 (21): 2129-39. Consequently, mutations of EGFR have been identified in several types of cancer, and it is the target of an expanding class of anticancer therapies.

The identification of EGFR as an oncogene has led to the development of anticancer therapeutics directed against EGFR, including gefitinib and erlotinib for lung cancer, and cetuximab for colon cancer.

Many therapeutic approaches are aimed at targeting EGFR. For example, Cetuximab and panitumumab are examples of monoclonal antibody inhibitors used to inhibit EGFR. However the former is of the IgG1 type monoclonal antibody, the latter of the IgG2 type monoclonal antibody; consequences on antibody-dependent cellular cytotoxicity can be quite different. Other monoclonals in clinical development that are directed towards EGFR include but are not limited to, zalutumumab, nimotuzumab, and matuzumab. These monoclonal antibodies inhibit EGFR by blocking the extracellular ligand binding domain. With the binding site blocked, signal molecules can no longer attach there and activate EGFR.

Another therapeutic approach is using small molecules to inhibit the EGFR tyrosine kinase, which act on the cytoplasmic side of the receptor. Without kinase activity, EGFR is unable to activate itself, which is a prerequisite for binding of downstream adaptor proteins. Ostensibly, the binding of the small molecules, halts the signaling cascade in cells that rely on this pathway for growth, tumor proliferation and migration is diminished. Examples of small molecule kinase inhibitors include but are not limited to, gefitinib, erlotinib, and lapatinib (mixed EGFR and ERBB2 inhibitor).

Cadherins (named for “calcium-dependent adhesion”) are a class of type-1 transmembrane proteins. They play important roles in cell adhesion, ensuring that cells within tissues are bound together. They are dependent on calcium (Ca²⁺) ions to function, hence their name. The cadherin superfamily includes cadherins, protocadherins, desmogleins, and desmocollins, and more. See Hulpiau P, van Roy F “Molecular evolution of the cadherin superfamily” (February 2009) Int. J. Biochem. Cell Biol. 41 (2): 349-69. In structure, they share cadherin repeats, which are the extracellular Ca²⁺-binding domains. There are multiple classes of cadherin molecule, each designated with a prefix (in general, noting the type of tissue with which it is associated). It has been observed that cells containing a specific cadherin subtype tend to cluster together to the exclusion of other types, both in cell culture and during development. For example, cells containing N-cadherin tend to cluster with other N-cadherin-expressing cells. However, it has been noted that the mixing speed in the cell culture experiments can have an effect on the extent of homotypic specificity. In addition, several groups have observed heterotypic binding affinity (i.e., binding of different types of cadherin together) in various assays. One current model proposes that cells distinguish cadherin subtypes based on kinetic specificity rather than thermodynamic specificity, as different types of cadherin homotypic bonds have different lifetimes.

Vimentin is a type III intermediate filament (IF) protein that is expressed in mesenchymal cells. IF proteins are found in all metazoan cells as well as bacteria. See Eriksson J E, Dechat T, Grin B, Helfand B, Mendez M, Pallari H M, Goldman R D (2009). “Introducing intermediate filaments: from discovery to disease.” J Clin Invest 119 (7): 1763-71. IF, along with tubulin-based microtubules and actin-based microfilaments, comprise the cytoskeleton. All IF proteins are expressed in a highly developmentally-regulated fashion; vimentin is the major cytoskeletal component of mesenchymal cells. Because of this, vimentin is often used as a marker of mesenchymally-derived cells or cells undergoing an epithelial-to-mesenchymal transition (EMT) during both normal development and metastatic progression.

A vimentin monomer, like all other intermediate filaments, has a central α-helical domain, capped on each end by non-helical amino (head) and carboxy (tail) domains. Two monomers are likely co-translationally expressed in a way that facilitates their formation of a coiled-coil dimer, which is the basic subunit of vimentin assembly. It has been used as a sarcoma tumor marker to identify mesenchyme.

The CD44 antigen is a cell-surface glycoprotein involved in cell-cell interactions, cell adhesion and migration. In humans, the CD44 antigen is encoded by the CD44 gene. See Spring F A, Dalchau R, Daniels G L, Mallinson G, Judson P A, Parsons S F, Fabre J W, Anstee D J (May 1988). “The Ina and Inb blood group antigens are located on a glycoprotein of 80,000 MW (the CDw44 glycoprotein) whose expression is influenced by the In(Lu) gene”. Immunology 64 (1): 37-43. CD44 is expressed in a large number of mammalian cell types. The standard isoform, designated CD44s, comprising exons 1-5 and 16-20 is expressed in most cell types. CD44 splice variants containing variable exons are designated CD44v. Some epithelial cells also express a larger isoform (CD44E), which includes exons v8-10.

CD44 is a receptor for hyaluronic acid and can also interact with other ligands, such as osteopontin, collagens, and matrix metalloproteinases (MMPs). CD44 function is controlled by its posttranslational modifications. One critical modification involves discrete sialofucosylations rendering the selectin-binding glycoform of CD44 called HCELL (for Hematopoietic Cell E-selectin/L-selectin Ligand). The HCELL glycoform was originally discovered on human hematopoietic stem cells and leukemic blasts, (see Dimitroff C J, Lee J Y, Rafii S, Fuhlbrigge R C, Sackstein R (June 2001) “CD44 is a major E-selectin ligand on human hematopoietic progenitor cells”. J. Cell Biol. 153 (6): 1277-86.) and was subsequently identified on cancer cells. See Burdick M M, Chu J T, Godar S, Sackstein R (May 2006). “HCELL is the major E- and L-selectin ligand expressed on LS174T colon carcinoma cells” J. Biol. Chem. 281 (20): 13899-905. HCELL functions as a “bone homing receptor”, directing migration of human hematopoietic stem cells and mesenchymal stem cells to bone marrow. Ex vivo glycan engineering of the surface of live cells has been used to enforce HCELLexpression on any cell that expresses CD44. CD44 glycosylation also directly controls its binding capacity to fibrin and immobilized fibrogen.

The CD44 protein participates in a wide variety of cellular functions including lymphocyte activation, recirculation and homing, hematopoiesis, and tumor metastasis. Transcripts for this gene undergo complex alternative splicing that results in many functionally distinct isoforms, however, the full length nature of some of these variants has not been determined. Alternative splicing is the basis for the structural and functional diversity of this protein, and may be related to tumor metastasis. Splice variants of CD44 on colon cancer cells display sialofucosylated HCELL glycoforms that serve as P-selectin, L-selectin, and E-selectin ligands and fibrin, but not fibrinogen, receptors under hemodynamic flow conditions pertinent to the process of cancer metastasis. CD44 gene transcription is at least in part activated by beta-catenin and Wnt signalling (also linked to tumour development).

HER2/neu (also known as ErbB-2) stands for “Human Epidermal growth factor Receptor 2” and is a protein giving higher aggressiveness in breast cancers. HER2 is a cell membrane surface-bound receptor tyrosine kinase and is normally involved in the signal transduction pathways leading to cell growth and differentiation. It is a member of the ErbB protein family, more commonly known as the epidermal growth factor receptor family. HER2/neu has also been designated as CD340 (cluster of differentiation 340) and p185. It is encoded by the ERBB2 gene. HER2 is thought to be an orphan receptor, with none of the EGF family of ligands able to activate it. However, ErbB receptors dimerise on ligand binding, and HER2 is the preferential dimerisation partner of other members of the ErbB family.

Estrogen receptor refers to a group of receptors that are activated by the hormone17β-estradiol (estrogen). See Dahlman-Wright K, Cavailles V, Fuqua S A, Jordan V C, Katzenellenbogen J A, Korach K S, Maggi A, Muramatsu M, Parker M G, Gustafsson J A (2006) “International Union of Pharmacology.LXIV.Estrogen receptors”. Pharmacol. Rev. 58 (4): 773-81. Two types of estrogen receptor exist: ER, which is a member of the nuclear hormone family of intracellular receptors, and the estrogen G protein-coupled receptor GPR30 (GPER), which is a G protein-coupled receptor. The following refers to the nuclear hormone receptor ER. The main function of the estrogen receptor is as a DNA-binding transcription factor that regulates gene expression. However, the estrogen receptor has additional functions independent of DNA binding.

There are two different forms of the estrogen receptor, usually referred to as alph (α) and beta (β), each encoded by a separate gene (ESR1 and ESR2 respectively). Hormone-activated estrogen receptors form dimers, and, since the two forms are coexpressed in many cell types, the receptors may form ERα (αα) or ERβ (ββ) homodimers or ERαβ (αβ) heterodimers. Estrogen receptor alpha and beta show significant overall sequence homology, and both are composed of five domains, known as the, A/B, C, D, E and F domains. The domain structures of ERα and ERβ, including some of the known phosphorylation sites involved in ligand-independent regulation. The N-terminal A/B domain is able to transactivate gene transcription in the absence of bound ligand (e.g., the estrogen hormone). While this region is able to activate gene transcription without ligand, this activation is weak and more selective compared to the activation provided by the E domain. The C domain, also known as the DNA-binding domain, binds to estrogen response elements in DNA. The D domain is a hinge region that connects the C and E domains. The E domain contains the ligand binding site as well as binding sites for coactivator and corepressor proteins. The E-domain in the presence of bound ligand is able to activate gene transcription. The C-terminal F domain function is not entirely clear and is variable in length.

Due to alternative RNA splicing, several ER isoforms are known to exist. At least three ERalpha and five ERbeta isoforms have been identified. The ERbeta isoforms receptor subtypes can transactivate transcription only when a heterodimer with the functional ERβ1 receptor of 59 kDa is formed. The ERβ3 receptor was detected at high levels in the testis. The two other ERalpha isoforms are 36 and 46 kDa. Both ERs are widely expressed in different tissue types, however there are some notable differences in their expression patterns: The ERα is found in endometrium, breast cancer cells, ovarian stroma cells, and the hypothalamus. In males, ERα protein is found in the epithelium of the efferent ducts. The expression of the ERβ protein has been documented in kidney, brain, bone, heart, lungs, intestinal mucosa, prostate, and endothelial cells. The ERs are regarded to be cytoplasmic receptors in their un-bound state, but visualization research has shown that a fraction of the ERs resides in the nucleus. The “ERα” primary transcript gives rise to several alternatively spliced variants of unknown function.

Estrogen and the ERs have also been implicated in over 70% of breast cancers, ovarian cancer, colon cancer, prostate cancer, and endometrial cancer. Advanced colon cancer is associated with a loss of ERβ, the predominant ER in colon tissue, and colon cancer is treated with ERβ-specific agonists. See Harris H A, Albert L M, Leathurby Y, Malamas M S, Mewshaw R E, Miller C P, Kharode Y P, Marzolf J, Komm B S, Winneker R C, Frail D E, Henderson R A, Zhu Y, Keith J C “Evaluation of an estrogen receptor-beta agonist in animal models of human disease” (2003) Endocrinology 144 (10): 4241-9.

In another embodiment, DNA binding dyes are used to determine aneuploidy or cancer state. Example od DNA binding dye include but are not limited to, EMA, DAPI, SYTOX®Blue, SYTOX®Red, SYTOX®Green, 7-AAD, Phenanthridiniums (propidium iodide, ethidium bromide), Acridines (acradine orange, proflavine HCl), actinomycins (7-actinomycin D), Antracyclines (daunomycin), Chromomycinones (mithramycin, chromomycin, olivomycin), and Bisbenzimadazoles (Hoechst 33258 and 33342). See Telford, W., Cytometry 13:137-143 (1992).

In one embodiment, 4′,6-diamidino-2-phenylindole (DAPI) is used to measure the state of the cellular DNA as an indicator for cancer state. DAPI is a fluorescent stain that binds strongly to A-T rich regions in DNA. It is used extensively in fluorescence microscopy. DAPI can pass through an intact cell membrane therefore it can be used to stain both live and fixed cells, though it passes through the membrane less efficiently in live cells and therefore the effectiveness of the stain is lower.

DAPI was first synthesized in 1971 in the laboratory of Otto Dann as part of a search for drugs to treat trypanosomiasis although it was unsuccessful as a drug. Further investigation indicated it bound strongly to DNA and became more fluorescent when it did so. This led to its use in identifying mitochondrial DNA in ultracentrifugation in 1975, the first recorded use of DAPI as a fluorescent DNA stain. See Kapuscinski, J., “DAPI: a DNA-specific fluorescent probe” (September 1995) Biotech Histochem 70 (5): 220-33.

Strong fluorescence when bound to DNA led to the rapid adoption of DAPI for fluorescent staining of DNA for fluorescence microscopy. Its use for detecting DNA in plant, metazoa and bacteria cells and virus particles was demonstrated in the late 1970s, and quantitative staining of DNA inside cells was demonstrated in 1977. Use of DAPI as a DNA stain for flow cytometry was also demonstrated around this time.

When bound to double-stranded DNA, DAPI has an absorption maximum at a wavelength of 358 nm (ultraviolet) and its emission maximum is at 461 nm (blue). Therefore for fluorescence microscopy DAPI is excited with ultraviolet light and is detected through a blue/cyan filter. The emission peak is fairly broad DAPI will also bind to RNA, though it is not as strongly fluorescent. Its emission shifts to around 500 nm when bound to RNA.

DAPI (magenta) bound to the minor groove of DNA (green and blue). From PDB 1D30. DAPI's blue emission is convenient for microscopists who wish to use multiple fluorescent stains in a single sample. There is some fluorescence overlap between DAPI and green-fluorescent molecules like fluorescein and green fluorescent protein (GFP) but the effect of this is small. Use of spectral unmixing can account for this effect if extremely precise image analysis is required.

Outside of analytical fluorescence light microscopy DAPI is also popular for labeling of cell cultures to detect the DNA of contaminating mycoplasma or virus. The labelled mycoplasma or virus particles in the growth medium fluoresce once stained by DAPI making them easy to detect.

DAPI can be used for both fixed and live cell staining, though the concentration of DAPI needed for live cell staining is generally much higher than for fixed cells. Zink D, Sadoni N, Stelzer E. “Visualizing Chromatin and Chromosomes in Living Cells.” (2003) Methods 29 (1): 42-50. It is labeled non-toxic in its MSD and though was not shown to have mutagenicity to E. coli it is labeled as a known mutagen in manufacturer information. As it is a DNA binding compound it is likely to have some low level mutagenic properties and care should be taken in its handling and disposal.

Epithelial cells stained with DAPI (blue) and two antibodies (green and red) via immunofluorescence. The Hoechst stains are similar to DAPI in that they are also blue-fluorescent DNA stains which are compatible with both live- and fixed-cell applications.

Other reagents which measure apoptosis and signaling are discussed below.

C. Isolation in Whole Blood

In one embodiment, the cells are isolated in whole blood to maintain the cellular environment that may affect the cell. Methods for analyzing CTCs have been proposed which separate them from blood. See for example, RosetteSep® made by StemCell Technologies. See Example 9 for an example. However, one embodiment of the present invention tests CTC, or other cells, in whole blood. For example, after collection, the blood could be prepared by reagents for the SCNP process (see U.S. Ser. No. 12/432,239). The blood can be contacted with the appropriate modulators soon after collection and then treated with other reagents in the process. Red blood cells may be lysed and the cells of interest can be fixed for subsequent analysis. At this point, the cells may be separated using an antibody labeled magnetic bead with washing to remove the cells of interest. In one embodiment, the cells are processed in a flow cytometer. An example of the time to process 0.05 mL blood is 0.33 minutes and up to 33.33 minutes for 5 mL blood. Recent advances in flow cytometry have shortened the time. For example, the Attune acoustic focusing cytometer (Life Technologies, Carlsbad, Calif.) is able to process cells more quickly.

In one embodiment, subsequent analysis with cell markers and gating or cell isolation techniques, allows for identification of the cells of interest and further analysis of their properties. One example of a method to test for CTCs or other cells is the following. Use whole blood and exclude hematopoietic cells from epithelial cells using CD45. Use tumor type specific markers to separate tumor cells, such as CTCs from other cells: Prostate specific antigen is one example. Use epithelial markers to separate normal from tumor cells: EpCAM, Cytokeratins (CK 7, 8, 18, 19, 20). However, aggressive or metastatic tumor cells can undergo an epithelial to mesenchymal transition (EMT) which facilitates migration and invasion into secondary metastatic sites. Therefore, there is lower level cell adhesion marker expression, altered morphology, gain or increase of mesenchymal marker protein expression and a loss or reuction of epithelial marker expression. So, epithelial markers may need to be interpreted in light of this observation. In other embodiments, it is useful to undergo sample enrichment such that the cells of interset are deliberately concentrated or the undesired cells are filtered out. One embodiment cell isolation or gating can use positive or negative selection, density gradient and/or cell size filtration. By way of example only, in one embodiment there is positive selection of EGFR expression using anti-EpCAM antibodies attached to magnetic beads. In another embodiment there is negative selection, such as using anti-CD45 attached to beads to filter out cell that express CD45.

The invention also provides for statistical methods to be applied to various aspects of the invention. In some embiodments, statistical methods may also be employed to select cells of interest. For example, one statistical method can be the following: R=(100/CV)², where R is the number of events meeting the required criteria for a CTC. The CV value is from a known control. Also, the statistical variation expected around the “true” value must be taken into account (SD). Example: if desired CV is 10% then r=100 events. The true value actually be somewhere between 80-120 events in 100,000 total events, if the estimated frequency was 1 in 1000. Low-normal leukocyte range is 5×10⁹/L. 10 mL blood=5×10⁷ leukocytes. If 1 in 10 million then 10 mL of blood would contain 50 CTCs. Other factors influencing stats: recovery/preservation of CTCs during sample prep and enrichment, intra operator/interlab variability. In one embodiment, methods can be used to facilitate identification of cells of interest. For example, flow cytometry can be employed as it has high specificity, but has a lower sensitivity (1 in 100,000). But newer cytometers with high flow rates can improve this rate, for example, the Accutune. In comparison, PCR can be used as it has high sensitivity (1 in 1 million), but amplification can result in false positives. In some embodiments identification of cells of interest can be preformed by flow cytometry. In other embodiments cell identification can be performed by immunochromatography. Immunochromatography. In other embiodments cell identification can be performed by immunohistochemistry in conjuction with laser-capture microdissection. In other embiodments cell identification can ber performed by high content cell screening.

D. Cell health

“Cell health” can be measured by assaying for cell viability e.g., by measuring cell membrane integrity and/or assaying one or more markers of apoptosis, necrosis, and/or autophagy (see PCT App. No. WO/2012/024546).

A cell health marker can be an apoptosis, necrosis, and/or autophagy marker. A cell health marker can be cell viability. In one embodiment, cell viability is not a cell health marker. A cell health marker can be an intracellular cell health marker. A cell health marker can precede the loss of cellular membrane integrity in the process of cell death in the cell.

A healthy cell can be a cell that lacks a cell health marker. A healthy cell can be cell that is viable and lacks an apoptosis, necrosis, and/or autophagy marker. A healthy cell can be a cell that comprises an intact cell membrane and lacks an apoptosis, necrosis, and/or autophagy marker. A healthy cell can be a viable, nonapoptic cell. For the purpose of this definition, a healthy cell can include an otherwise diseased cell that does not have these markers.

An unhealthy cell can be a cell that comprises a cell health marker. An unhealthy cell can be a cell that comprises one or more apoptosis, necrosis, and/or autophagy markers. An unhealthy cell can be a nonviable cell. An unhealthy cell can be a viable cell that comprises one or more apoptosis, necrosis, and/or autophagy markers. An unhealthy cell can be a cell that comprises an intact cell membrane and comprises one or more cell health markers. An unhealthy cell can be a cell that comprises an intact cell membrane and comprises one or more apoptosis, necrosis, and/or autophagy markers. In one embodiment, an unhealthy cell is a cell undergoing apoptosis. In one embodiment, the cell undergoing apoptosis can have an intact cell membrane. In one embodiment, the cell undergoing apoptosis is a viable cell.

In one embodiment, a method is provided to detect and exclude unhealthy (e.g., apoptotic cells) from an analysis, e.g., a cell signaling profile analysis, e.g., measurement of the activation level of an activatable element. In one embodiment, a method is provided to differentiate between healthy, non-apoptotic leukemia cells and unhealthy, apoptotic leukemia cells. Use of the method does not require that the cells be diseased, such as a leukemia cell. Rather, the method can be used to examine whether cells are unhealthy (e.g., apoptotic) or healthy (e.g., nonapoptotic).

Apoptosis is a complex pathway in which cells undergo cell death. There are methods, indicators and reagents to detect cells that are undergoing apoptosis. Examples of apoptosis markers include, e.g., caspases, caspase cleavage products such as fluorogenic dye caspase substrates, c-PARP, cleaved cytokeratin 18, cleaved caspases, cleaved caspase 3, cytochrome C, apoptosis inducing factor (AIF), Inhibitor of Apoptosis (IAP) family members, Bcl-2 family members (e.g., anti-apoptotic proteins (e.g., MCL-1, BCL-2, BCL-XL), BH3-only apoptotic sensitizers (e.g., PUMA, NOXA, Bim, Bad), pro-apoptotic proteins (e.g., Bad, Bax), p53, c-myc proto-oncogene, APO-1/Fas/CD95, growth stimulating genes, or tumor suppressor genes. In one embodiment, the presence a molecule (e.g., c-PARP) in a cell is an apoptotic and/or necrosis marker. In another embodiment, the absence of a molecule in a cell is an apoptotic and/or necrosis marker. In another embodiment, an apoptosis and/or necrosis marker is an intracellular apoptosis and/or necrosis cell marker.

In one embodiment, a cell health marker is a necrosis marker. In one embodiment, the necrosis marker includes, e.g., beta-glucuronidase, BV2, cardiac troponin, cardiac troponin I, C-reactive protein, creatine kinase MB (CPK-MB), Factor VIII procoagulant, H-FAMP (heart-type fatty acid-binding protein), HNK1-, isocitrate dehydrogenase (ICDH), IL-6, IL-18, lactate dehydrogenase, myoglobin, myosin, procalcitonin, serum immunoreactive prolyl 4-hydroxylase (S-IRPH), tenascin-C, TNF-alpha, TNF-R1, troponin I, and troponin T.

In one embodiment, a cell health marker is an autophagy marker. The autophagy can be macro-autophagy, micro-autophagy, and chaperone-mediated autophagy. In one embodiment, the autophagy marker includes, e.g. AMPK, mTOR, ULK1, ULK2 and other Atg family members (e.g., ATG16L), PIK3C3, BECN1, Vps34, Beclin-1, MAP1LC3A,B,C, GABARAP, GABARAPL1, GABARAPL2, UVRAG, IRGM, CLN3, Parkin, p62, and LAMP2 and other autophagy proteins as described in Behrends, Harper et al. “Network organization of the human autophagy system” Nature. 2010 Jul. 1; 466(7302):68-76 and Glick, Macleod et al. Autophagy: cellular and molecular mechanisms. J. Pathol. 2010 May; 221(1):3-12.

In one embodiment, the one or more cell health markers, e.g., one or more intracellular cell health markers, can be one or more activatable elements. In some embodiments, the one or more cell health markers can comprise 1, 2, 3, 4, 5, 6, 7, 8, 9, or more cell health markers, e.g., intracellular cell health markers. In another embodiment, the one or more cell health markers, e.g., intracellular cell health markers, can comprise 1-10, 1-5, 2-10, 2-7, or 2-5 cell health markers. In another embodiment, the cell health marker is an extracellular marker.

Reagents that can be used to determine whether a cell is undergoing apoptosis include, e.g., mitochondrial membrane dyes, Annexin-V, 7-AAD (7-aminoactinomycin), Amine Aqua, trypan blue, propidium iodide or other viability dyes and other compounds that are recited below or are in the cell apoptosis pathway.

Cells can become unhealthy (e.g., have one or more cell health markers, e.g., one or more apoptosis, necrosis, and/or autophagy markers) over time due to sample treatment, removal from the body, storage, etc. For example, cells in a sample can display one or more cell health markers, e.g., one or more intracellular cell health markers, over time due to the means by which the cells are handled. For example, sample treatment, removal of the cells from the body, storage of the cells, etc., can cause a cell to display one or more cell health markers. The one or more cell health markers can be one or more intracellular cell health markers.

The percentage of healthy cells in a sample can decrease over time due to sample treatment, removal from the body, storage, etc. Due to the practicalities of sample collection, cells may be frozen for transport and/or storage prior to assaying by a functional assay, e.g., single cell network profiling (SCNP), as described below. In one embodiment, a sample of frozen cells is thawed, the cells are stimulated with a modulator, fixed, permeabilized, stained with fluorophore-conjugated antibodies that bind a phosphorylated protein, and then analyzed by flow cytometry. After thawing, the percentage of unhealthy cells (e.g., cells with a cell health marker) in a sample may increase. Some cells may become unhealthy (e.g., display one or more apoptosis, necrosis, and/or autophagy markers) that may affect the results of an assay, e.g., a measurement of the activation level of an activatable element. In one embodiment, a method is provided that accounts for the affect on the activation level of the activatable element of the unhealthy cells.

In one embodiment, an unhealthy cell is a nonviable cell. Cell viability can be determined by analyzing cells by dye exclusion, by profiling DNA content, and by assaying for morphological changes. In one embodiment, a DNA stain can be used to analyze cells by dye exclusion, by profiling DNA content, or by assaying for morphological changes.

In one embodiment, cell viability is determined by determining the integrity of the membrane of a cell. A cell can be considered dead (nonviable) when the plasma membrane of the cell has lost its integrity. When a cell plasma membrane is intact, an exclusion dye cannot cross the intact cell plasma membrane. When a cell plasma membrane is not intact, an exclusion dye can cross the plasma membrane. In one embodiment, cell membrane integrity can be determined with a cell exclusion dye. In one embodiment, a cell that does not stain with a cell exclusion dye is a viable cell. In another embodiment, a cell that is stained with a cell exclusion dye is a nonviable cell. In one embodiment, the cell exclusion dye is trypan blue, Eosin, 7-AAD, Amine Aqua, an amine-reactive fluorescent dye, erythrosine, or propidium iodide.

In another embodiment, cell membrane integrity can be assayed by assaying for DNA content of a cell. If a cell is permeabilized, low molecular weight DNA can leak out. In one embodiment, measuring the DNA content of a cell with a DNA stain can be used to determine if a cell is nonviable. Since a non-viable cell, (which can be a fixed or permeabilized cell) can have less DNA than a viable cell, a non-viable cell can contain less DNA staining when DNA of the cell is stained using, e.g., a fluorochrome. A cell with lower DNA staining than that of a cell in the G1 phase of the cell cycle (cells in so called “sub-G1 peaks” or “G0” cells of a flow cytometry analysis) can be considered apoptotic or non viable. The distinction between apoptotic and non-viable could be further determined in combination with a fixation insensitive viability dye such as Amine Aqua or equivalent amine reactive dye which survives the fixation process. The reduction in staining/DNA content of these cells can be measured by flow cytometry or by analyzing fixed cells.

A G2-phase cell can exhibit a reduced DNA content, but such a cell can be indistinguishable from a G1-phase cell that has similar DNA content of a G1-cell in a flow cytometry analysis. Therefore, an apoptotic G2-phase cell may not be detected as apoptotic, e.g., in a flow cytometry analysis. The inability to distinguish an apoptotic G2-phase cell from a G1-cell in a sample of cells can result in an underestimation of the apoptotic population of cells in the sample of cells.

Assays that examine chromatin morphology can be used to distinguish between viable and nonviable cells. Hoechst 33342 is a nucleic acid stain. Acridine orange (Hoechst 33342) plus DRAQ5 can penetrate the plasma membrane of a cell and stain DNA in the cell without permeablization of the membrane of the cell. In contrast to the nuclei of normal cells, the nuclei of apoptotic cells have highly condensed chromatin that can be uniformly stained by Hoechst 33342. DRAQ5 can also measure DNA content of live cells and be used to identify and potentially exclude unhealthy cells with lower “sub-G1 peaks” from a live cell assay.

The percent of viable cells in a sample can decrease over time due to sample treatment, removal from the body, storage, etc. Due to the practicalities of sample collection, cells may be frozen for transport and/or storage prior to assaying by a functional assay, e.g., single cell network profiling (SCNP), as described below. In one embodiment, a sample of frozen cells is thawed, the cells are stimulated with a modulator, fixed and permeabilized, the cells are stained with fluorophore-conjugated antibodies that bind a phosphorylated protein, and the cells are then analyzed with flow cytometry. After thawing, the percentage of viable cells (cells without an intact cell membrane) in a sample may decrease.

E. Cell Health Markers

In one embodiment, an unhealthy cell is a cell undergoing apoptosis or cell mediated cytotoxicity. A cell undergoing apoptosis or cell mediated cytotoxicity can be characterized by cleavage of genomic DNA into discrete fragments prior to membrane disintegration of the cell. Genomic DNA can be assayed in at least the following ways: by assaying for apoptotic DNA “ladders” (with 180 bp multiples as “rungs” of the ladder) derived from a population of cells, or by quantification of histone complexed DNA fragments with an ELISA, or by quantifying total cellular DNA Content. For more information on apoptosis and DNA damage (see WO/2012/024546).

In one embodiment, a cell health marker is a caspase. Caspases can be indicators of cell health. Proteases can be involved in the early stages of apoptosis. The appearance of caspases can set off a cascade of events that can disable a multitude of cell functions. Caspase activation can be analyzed in different ways, such as by an in vitro enzyme assay, e.g., active Caspase 3 in cellular lysates. They may also be analyzed by detection of an in vivo caspase substrate. For instance, caspase 3 can be activated during early stages of apoptosis. A caspase 3 substrate is PARP (Poly-ADP-Ribose-Polymerase), and c-PARP can be detected with an anti-PARP antibody. Caspases can also be analyzed by M30 CytoDeath (available from Hoffman La Roche), which measures caspase-cleaved cytokeratin 18 (not detectable in native CK18 of normal cells) with M30 antibody. Some caspase cleavage dyes fluoresce when cleaved. Such dyes can be added to a cell, be cleaved in vivo, and fluorescence can be detected. An example of a caspase cleavage dye is CellEvent Caspase-3/7 Green Detection Reagent from Invitrogen, which is an intrinsically non-fluorescent as a four amino acid peptide (DEVD) conjugated to a nucleic acid binding dye. The DEVD sequence can be a cleavage site for caspase-3/7 and the conjugated dye can be non-fluorescent until it is cleaved from the peptide and bound to DNA.

In another embodiment, a cell health marker is the release of cytochrome C and AIF (apoptosis inducing factor) into the cytoplasm by mitochondria. During apoptosis, mitochondrial permeability can be altered and apoptosis specific protease activators can be released from mitochondria. Specifically, the discontinuity of the outer mitochondrial membrane results in the redistribution of cytochrome C to the cytosol followed by subsequent depolarization of the inner mitochondrial membrane. Cytochrome C (Apaf-2) release further promotes caspase activation by binding Apaf-1 and therefore activating Apaf-3 (caspase 9). AIF released in the cytoplasm can have proteolytic activity and can by itself be sufficient to induce apoptosis.

In another embodiment, a cell health marker, e.g., an early indicator of apoptosis in mammalian cells, is the loss of the phospholipid membrane asymmetry of the cell. This results in exposure of phosphatidylserine on the outer surface of the plasma membrane. The change in membrane asymmetry can be analyzed using Annexin V antibody binding followed by quantification with flow cytometry. An assay using Annexin V can be an extracellular assay.

In another embodiment, a cell health marker is an apoptosis related protein. Cell health may be determined by the detection of apoptosis related proteins. These proteins include: the Bcl-2 protein family (increased apoptosis with high Bax levels or low Bcl-2 levels); p53; c-myc proto-oncogene; cell surface receptors (such as APO-1/Fas/CD95 for example); growth stimulating genes (such as Ras for example); and tumor suppressor genes (such as Rb for example). Another embodiment includes measuring changes in surface or lineage markers such as CD45, CD34, and CD11b, further proteins are listed in WO/2012/024546.

One embodiment measures sample viability or cell health and calculates the sample's “percent healthy” (percentage of healthy viable, non-apoptotic cells) to exclude samples of low sample viability or cell health from further analysis. In one embodiment, a threshold of percent healthy is used, such as about 5%, 10%, 20%, 25%, or 30%, as a cut off for the percentage of healthy cells in a cell sample required for that sample to be further analyzed for signaling biology and other cellular readouts. For example, if less than about 5%, 10%, 20%, 25%, or 30% of the cells in a sample are healthy, the sample can be excluded from an analysis, e.g., an analysis of signaling biology and other cellular readouts. If more than about 5%, 10%, 20%, 25%, or 30% of the cells in a sample are healthy, the sample can be included in an analysis e.g., an analysis of signaling biology and other cellular readouts. In one embodiment, a threshold is used of about 35%, 40%, 45%, 50%, 55% or 60% as the cutoff prior to measuring signaling biology and other cellular readouts as shown below. For example, if less than about 35%, 40%, 45%, 50%, 55% or 60% of the cells in a sample are healthy, the sample can be excluded from an analysis, e.g., an analysis of signaling biology and other cellular readouts. If more than about 35%, 40%, 45%, 50%, 55% or 60% of the cells in a sample are healthy, the sample can be included in an analysis, e.g., an analysis of signaling biology and other cellular readouts. In another embodiment, the threshold is about 65%, 70%, 75%, 80%, 85%, 90%, or 95%. For example, if less than about 65%, 70%, 75%, 80%, 85%, 90%, or 95% of the cells in a sample are healthy, the sample can be excluded from an analysis, e.g., an analysis of signaling biology and other cellular readouts. If more than about 65%, 70%, 75%, 80%, 85%, 90%, or 95% of the cells in a sample are healthy, the sample can be included in an analysis e.g., an analysis of signaling biology and other cellular readouts.

In one embodiment, a sample of cells is excluded (or not included) in an analysis if the percentage of nonviable cells in the sample is above a threshold. In one embodiment, the threshold is about 5%, 10%, 15%, 20%, 25%, 30%, 35%, 40%, 45%, or 50% nonviable cells. In one embodiment, a sample of cells is included in an analysis if the percentage of viable cells in the sample is below a threshold. In one embodiment, the threshold is about 95%, 90%, 85%, 80%, 75%, 70%, 65%, 60%, 55%, 50%, 45%, 40%, 35%, 30%, 25%, 20%, 15%, 10%, or 5% viable cells in a sample.

In another embodiment, a cell in a sample can be individually gated from an analysis based on the viability and/or health of the cell. In another embodiment, a cell in a sample can be gated from an analysis if the cell is viable and/or healthy. In another embodiment, a cell can be included in an analysis if the cell is viable and/or healthy. In another embodiment, a cell can be excluded from an analysis if the cell is not viable and/or not healthy.

A measurement of sample viability or cell health may occur at the beginning of an experiment, which can be after a ficoll separation and rest step. This time period may be called time 0 in an experiment with no modulator is applied to a sample of cells or time 15 minutes in an experiment where a modulator is applied to a sample of cells. Sample viability or cell health may also be measured at later timepoints in an experiment, such as after about 5 min, 10 min, 15 min, 20 min, 25 min, 30 min, 35 min, 40 min, 45 min, 50 min, 55 min, 1 hr, 2 hr, 3 hr, 4 hr, 5 hr, 6 hr, 7 hr, 8 hr, 9 hr, 10 hr, 11 hr, 12 hr, 13 hr, 14 hr, 15 hr, 16 hr, 17 hr, 18 hr, 19 hr, 20 hr, 21 hr, 22 hr, 23 hr, 24 hr, 30 hr, 36 hr, 40 hr, 48 hr, 72 hr, or more or with incubation with a modulator. Sample viability or cell health may be measured after about 5 min to 48 hr, 15 min to 48 hr, 30 min to 48 hr, 1 hr to 48 hr, 1 hr to 36 hr, 1 hr to 24 hr, or 12 hr to 24 hr, or with incubation with a modulator.

F. Calculation of Percent Healthy Cells

One embodiment focuses on a cell population of interest. In one embodiment, the cells are gated for leukemic blasts (P1) and healthy cells in the blast population (Healthy P1). In one embodiment intact cells are identified using light scattering properties (Forward and Side Scatter), live cells are identified using Amine Aqua, leukemic blasts are identified using Side Scatter and CD45, and non-apoptotic leukemic blasts (Healthy P1) are identified by assaying for c-PARP. A higher range of induced signaling (e.g., of an activatable element) can occur when c-PARP+ cells are removed to make a healthy P1 population. G-CSF can be used as a modulator and p-STAT3 can be the activatable element. Percent healthy analysis can be performed by looking at PI3K/Ras signaling S6 as modulated by SCF or Flt3 Ligand.

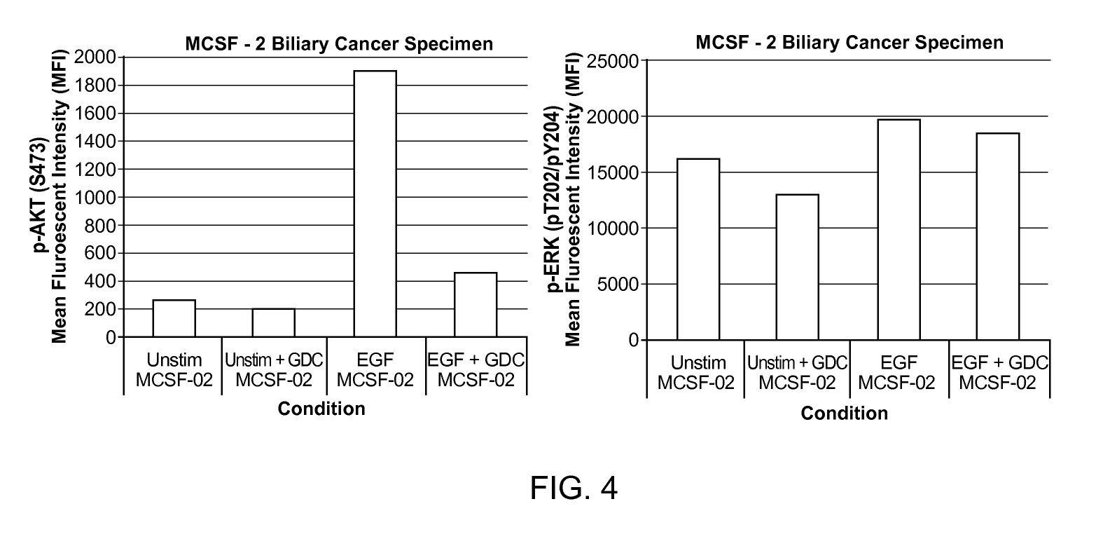

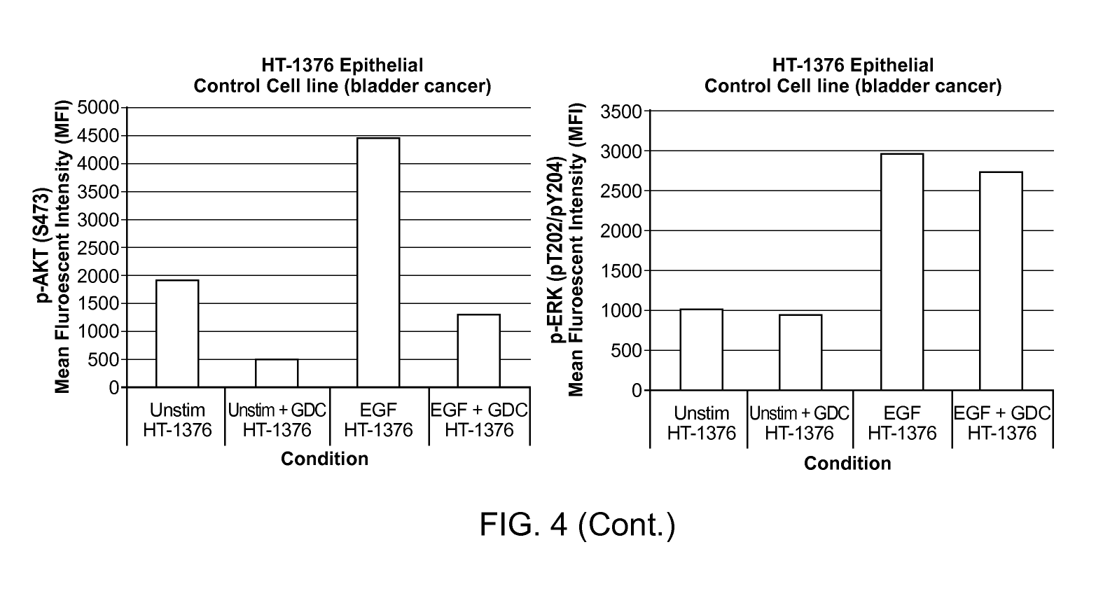

FIG. 4 of WO/2012/024546 shows that c-PARP cells can be refractory to signaling, and removal of c-PARP+ cells (via gating) can improve assay robustness. FIG. 4A of WO/2012/024546 shows SCNP flow cytometry contour plots from one leukapheresis mononuclear cell AML patient sample that is representative of the entire set. Cells were gated to remove non-viable cells (Amine Aqua+), and to remove debris. c-PARP (apoptotic) percentage is based on the viable, non-debris fraction. FIG. 4B of WO/2012/024546 shows contour plots of G-CSF induced p-STAT3 and p-STAT5 signaling from c-PARP cells (top row) or c-PARP^{neg} cells (bottom row). FIG. 4C of WO/2012/024546 shows the same two gating schemes for Pt.2 showing the basal and SCF stimulated median fluorescent intensity data for p-ERK. High statistical significant was only achieved by excluding c-PARP cells. FIG. 4D of WO/2012/024546 shows percent healthy cells versus Jak/Stat signaling.

In another embodiment, the method gates on a Healthy P1 sample, applies the appropriate preset value or threshold to remove unhealthy cells from the analysis, and then adjusts the signaling for the remaining cells, including the Healthy P1 cells, by the percent healthy. This method first removes the cells that are actually apoptosing (“unhealthy” cells) and then adjusts for the effects of those apoptosing cells on otherwise healthy cells. Otherwise healthy cells can have low signaling values because of an effect of an apoptosing cell on the otherwise healthy cells. Without wishing to be bound by theory, an apoptosing cell could affect the extracellular conditions of the healthy cells, which could affect the signaling of healthy cells. In some embodiments, samples with low percent healthy (for example, less than a preset value as recited herein, such as 25%) may be removed from the analysis even after gating for healthy cells. In another embodiment, gating does not occur before cell health analysis.

Multiple methods may be used to incorporate the cell health into the cell signaling data to improve the accuracy of predictive accuracy (measured by performance characteristic like AUC_{ROC}, Sensitivity, Specificity, Negative Predictive Value, Positive predictive value) of test classifiers. In one embodiment, cell health is determined directly for each cell. Then the cell may be excluded if the health is within or outside of a preset range, or above/below a certain threshold, or if the cell has a cell health marker, e.g., an intracellular cell health marker. Direct measurements may be made by measuring an apoptotic marker in each cell. One example of a cell health marker is the amount of c-PARP, but one or more other markers may be used. In one embodiment samples can be gated on cleaved PARP (c-PARP), allowing one to focus on the populations of c-PARP^{neg} and c-PARP⁺ cells. Since c-PARP cells (higher apoptosis) have reduced sensitivity to modulators, cells that are not gated and excluded may “dampen” a SCNP profile.

In another embodiment, MCL-1 can be used. Absence of MCL-1 is a measure (marker) of apoptosis and measurements above/below a certain threshold, or outside/within a certain range, can indicate cell health.

In another embodiment, cell health may be calculated for one cell population and applied to others in an indirect measurement. In one embodiment, the percentage of healthy cells in one sample of cells is used to adjust the measurement of an activation level of an activatable element for another sample of cells. The percent healthy calculation for AML samples, for example, can be obtained various ways, such as:

$$\frac{\# {PARP}^{neg} myeloid cells}{Total Events} or \frac{\# {PARP}^{neg} myeloid cells}{\# Intact cells} or$$

$$\frac{\# {PARP}^{neg} myeloid cells}{\# Intact myeloid cells}$$

where “Intact” is defined by a cell size and granularity gate (Forward and Side Scatter parameters), where Myeloid is defined by markers of myeloid cells (e.g., CD45 and Side Scatter, or other myeloid markers e.g., CD13, CD33), where PARP^{neg} cells are defined by c-PARP measurements within myeloid cells.

In one embodiment, the percentage of unhealthy cells in a sample is the percentage of cells in the sample with one or more cell health markers out the total number of cells in the sample. In another embodiment, the percentage of unhealthy cells in a sample is the percentage of cells in the sample with one or more intracellular cell health markers out the total number of cells in the sample. In another embodiment, the percentage of unhealthy cells in a sample is the percentage of cells in the sample with both one or more cell health markers and an intact cell membrane, out of the total number of cells with an intact cell membrane in the sample. These measures can be highly correlated.

A percent healthy calculation can be used to adjust one or more SCNP profiles for a patient, resulting in improvements in the predictive accuracy of the test classifiers (measured by performance characteristic like AUC_{ROC}, Sensitivity, Specificity, Negative Predictive Value, Positive predictive value). The adjustments to the SCNP profiles can be performed in two ways: by adjusting by residual or adjusting the population modeling. In one embodiment, adjusting by residuals comprises looking at a plot of induced signaling vs. percent healthy for a node (e.g., a modulator/activatable element pair). Generally, a plot might show that the induced signaling increases as the percent healthy increases. The data average can be fit with some linear trend. The residual can be found by measuring how far above or below the linear trend a single node data point lays. In another embodiment adjusting is by adjusting by population modeling. With regard to adjusting by population modeling, within a sample, since dead or apoptosing cells do not signal in response to certain modulators, dead or apoptosing cells (e.g., 40% of the sample) can have the same phospho-flow cytometry profile in multiple wells (e.g., multiple samples) even when treated with one or more modulators as compared to the cells that have not been treated. However, the remaining 60% healthy cells can have a different profile in different wells when treated with one or more modulators, depending on the modulator, as compared to the cells that have not been treated. Population modeling can be used to estimate the signal of the healthy cells within each examined node. The term “node” can describe a specific modulator/activatable element pair. Nodes can be represented using the notation “modulator→activatable element”. For example, IL-6→pStat5 represents the modulator IL-6 and the activatable element phosphorylated Stat5 (pStat5). Examples of activatable elements are described below.

The measurement of the percent healthy cells in a population of cells that do not change signaling profiles can be used to indirectly calculate the signaling profiles of cells that do change signaling profiles based upon varying stimulation conditions. Details of a calculation:

$$Fold = \frac{{Mean}_{stim}}{{Mean}_{unstim}}$$

Since Meanₛₜᵢₘ can be described by, (f_{Unhealthy}*Mean_{Unhealthy}+f_{healthy}*Mean_{healthy}) where Mean_{Unhealthy} is the mean for the cells that are undergoing apoptosis, Mean_{healthy} is the mean for the cells that are healthy, f_{Unhealthy} and f_{healthy} are the fraction of apoptosing and healthy cells. Since apoptosing cells are unlikely to change in level of signaling due to modulation Mean_{Unhealthy}=Meanᵤₙₛₜᵢₘ which can be known from a well with no modulation. The fold formula above can be written instead as:

Meanₛₜᵢₘ=f_{Unhealthy}*Meanᵤₙₛₜᵢₘ+f_{healthy}*Mean_{healthy}

The values of f_{healthy} and f_{Unhealthy}, the fraction of unhealthy and healthy cells, can be obtained from the percent healthy calculation within a population, leaving only Mean_{healthy} unknown and able to be calculated. The above formula can be applied within viable cells, within a specific cell population such as P1.

An apoptosis readout like c-PARP, when tested in combination with other SCNP readouts (e.g., p-Akt) in the same well, can be used to apply an adjustment for cell health at an individual cell level. For example, p-Akt readout for each cell can be weighted by the c-PARP signal for that cell, so that modulated levels of p-Akt from dying cells can be either increased or decreased when computing normalized metrics like ‘Fold’ change.

An adjustment for cell health at an individual cell level can be applied to an individual cell based on where the individual cell is on an apoptosis timeline (stage of a cell in apoptosis). An apoptosis timeline is described, for example, at: <<www.invitrogen.comisite/us/en/home/Products-and-Services/Applications/Cell-and-Tissue-Analysis/Flow-Cytometry/FC-Misc/Apoptosis.html.>> After induction of apoptosis, cell function changes can occur generally in the following order: decrease in mitochondrial membrane potential, mitochondrial transition pore opening, phosphatidylserine translocation to outer membrane, increase in caspase activity, decrease in metabolic activity, increase in DNA condensation, decrease in plasma membrane integrity, and increase in DNA fragmentation. Early and late markers of apoptosis can be used to detect events along a linear apoptosis continuum to determine how close a cell is to death. The activation status or activation level of an activatable element may be affected based on the position of the cell in the apoptosis timeline. For example, the activation level of an activatable element may decrease the closer the cell is to the end of the apoptosis timeline. Various factors can be employed to adjust the signaling level. For example, the presence of an early apoptosis marker, or even a pre-apoptosis marker (cell cycle arrest, DNA damage, presence of viral infection etc.), can indicate a small correction of cell signaling data (e.g., measurement of the activation level of an activatable element) is appropriate whereas the presence of a later apoptosis marker can indicate a larger correction of cell signaling data is appropriate, up to and including elimination of the cell from the signaling analysis. In one embodiment, an entire population of cells can be adjusted by a factor instead of individual adjustments to individual cells. In one embodiment, the activation level of an activatable element for an entire population of cells can be adjusted by a factor instead of individual adjustments to activation levels of activatable elements in individual cells.

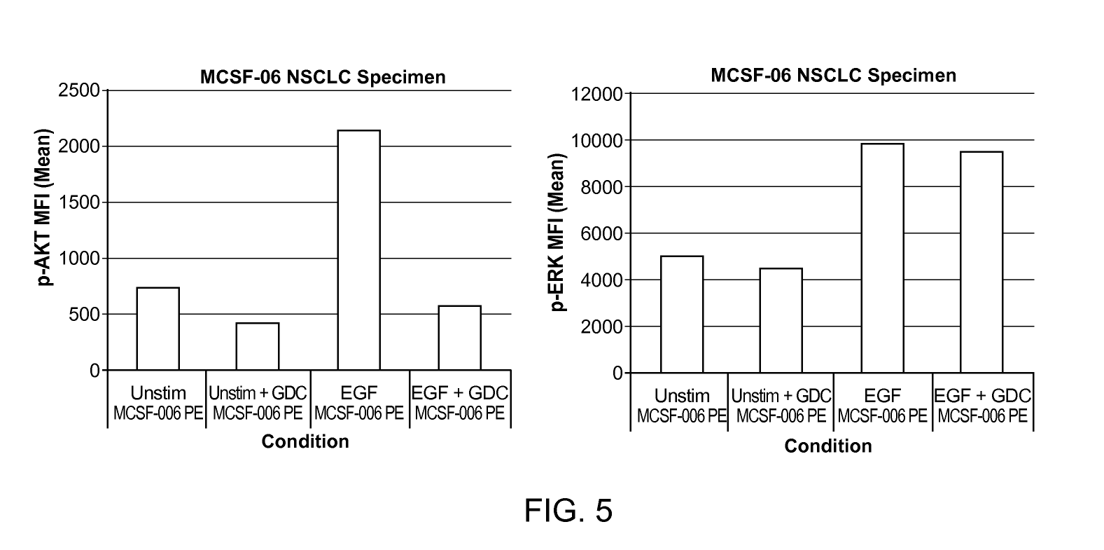

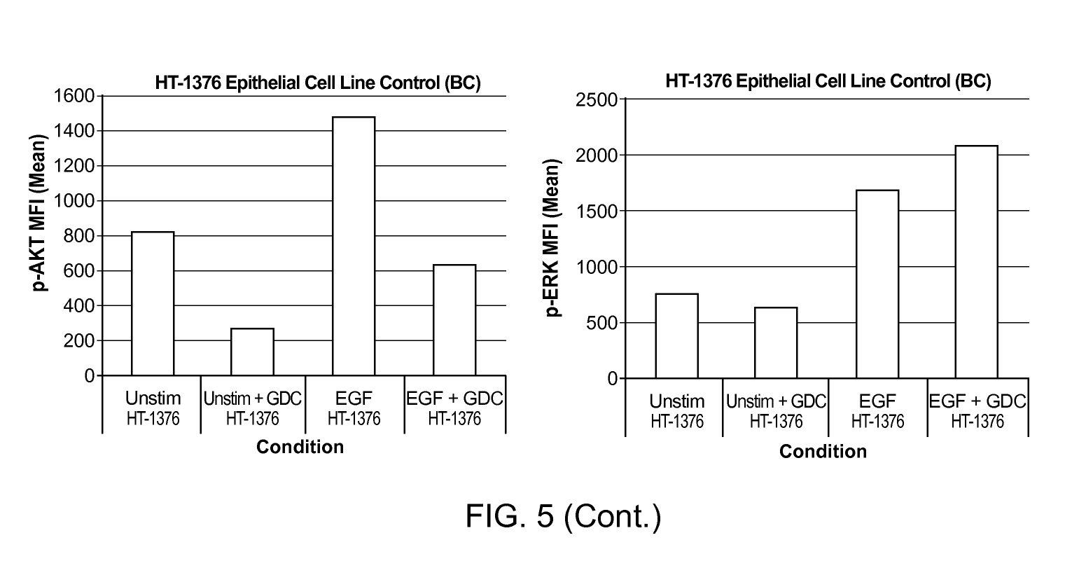

An alternative metric can be used to measure induced apoptosis at various time points, including experiment time zero. This alternative metric minimizes the effects of pre-existing sample viability, which can be affected by sample preparation, handling, cryopreservation and/or thawing before the experiment. This inherent variation in pre-existing sample viability can confound classification of samples. For example, the variability between samples may be due to sample handling and not true disease differences. The alternative metric normalizes the sample viability by dividing readouts for each sample, for each time point and modulated condition, by the associated readout for the un-stimulated condition at experimental time zero or at the matched experimental timepoint, e.g., see FIG. 5 of WO/2012/024546. An extension of this metric utilizes the normalized metrics but also integrates the various time points associated with the specific treatment (such as the use of an apoptosis inducing agent, such as etoposide) into a unified metric. This extended, and normalized, measurement can be designed to measure the total experimental effect of the specific treatment on the sample over the time points of the experiment. Time zero for testing may occur at 6 hours as the samples may need to be thawed and brought to a state ready for analysis.

The above calculations are also useful to select nodes that are less affected by apoptosis. For example, in one embodiment data are collected and then adjusted as set out above. Then, the adjusted values are plotted versus the unadjusted values to determine and the nodes are selected which are above the X/Y line.

G. Activatable Elements

The methods and compositions described herein may be employed to examine and profile the status or activation level of any activatable element in a cellular pathway, or collections of such activatable elements. Single or multiple distinct pathways can be profiled (e.g., sequentially or simultaneously), or subsets of activatable elements within a single pathway or across multiple pathways can be examined (e.g., sequentially or simultaneously).

In some embodiments, apoptosis, signaling, cell cycle and/or DNA damage pathways are characterized in order to classify one or more cells in an individual. The characterization of multiple pathways can reveal operative pathways in a condition that can then be used to classify one or more cells in an individual. In some embodiments, the classification includes classifying the cell as a cell that is correlated with a clinical outcome. The clinical outcome can be the prognosis and/or diagnosis of a condition, and/or staging or grading of a condition. In some embodiments, the classifying of the cell includes classifying the cell as a cell that is correlated with a patient response to a treatment. In some embodiments, the classifying of the cell includes classifying the cell as a cell that is correlated with minimal residual disease or emerging resistance. In some embodiments, the cell classification includes correlating a response to a potential drug treatment. See also U.S. Ser. Nos. 12/432,720 and 61/048,886 for activatable elements.

As will be appreciated by those in the art, a wide variety of activation events can find use in the methods described herein. In general, activation can result in a change in the activatable protein that is detectable by some indication (termed an “activation state indicator”), e.g., by altered binding of a labeled binding element or by changes in detectable biological activities (e.g., the activated state has an enzymatic activity which can be measured and compared to a lack of activity in the non-activated state). Using one or more detectable events or moieties, two or more activation states (e.g., “off” and “on”) can be differentiated.

The activation state of an individual activatable element can be in the on or off state. As an illustrative example, and without intending to be limited to any theory, an individual phosphorylatable site on a protein can activate or deactivate the protein. Phosphorylation of an adapter protein can promote its interaction with other components/proteins of distinct cellular signaling pathways. In another embodiment, the difference in enzymatic activity in a protein can reflect a different activation state. The terms “on” and “off,” when applied to an activatable element that is a part of a cellular constituent, can be used here to describe the state of the activatable element, and not the overall state of the cellular constituent of which it is a part.

The activation state of an individual activatable element can be represented as continuous numeric values representing a quantity of the activatable element or can be discretized into categorical variables. For instance, the activation state may be discretized into a binary value indicating that the activatable element is either in the on or off state. As an illustrative example, and without intending to be limited to any theory, an individual phosphorylatable site on a protein will either be phosphorylated and then be in the “on” state or it will not be phosphorylated and hence, it will be in the “off” state. See Blume-Jensen and Hunter, Nature, vol. 411, 17 (May 2001), pp. 355-365.

Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element can be measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell can be the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations. For more information on the measurement of activatable elements, specific activatable elements, signaling pathways, and drug transporters, see U.S. Ser. No. 61/350,864 or U.S. Pub. No. 2009/0269773.

In some embodiments, the activation levels of one or more activatable elements of a cell from a first population of cells and the activation levels of one or more activatable elements of a cell from a second population of cells are correlated with a condition. In some embodiments, the first and second homogeneous populations of cells are from solid tumor cell populations.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change can occur to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. The term “plurality” as used herein refers to two or more. In some embodiments, the activation level of at least about 2, 3, 4, 5, 6, 7, 8, 9, 10, or more than 10 intracellular activatable elements are determined.

Activation states of activatable elements can may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In some embodiments, the activatable element is a protein. Examples of proteins that can include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Pub. No. 2006/0073474 entitled “Methods and compositions for detecting the activation state of multiple proteins in single cells” and U.S. Pub. No. 2005/0112700 entitled “Methods and compositions for risk stratification”. See also U.S. Ser. Nos. 61/048,886, 61/048,920 and Shulz et al., Current Protocols in Immunology (2007) 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, Ets-1, Ets-2, SPDEF, GABPα, Tel, Tel2, WT-1, HMGA, pS6, 4EPB-1, eIF4E-binding protein, RNA polymerase, initiation factors, elongation factors.

In some embodiments, the methods described herein are employed to determine the activation level of an activatable element, e.g., in a cellular pathway. Methods and compositions are provided for the determination of a cell signaling profile (e.g., activation level of an activatable element) of a cell according to the activation level of an activatable element in a cellular pathway. Methods and compositions are provided for the determination of the cell signaling profile of a cell in a first cell population and a cell in a second cell population according to the activation level of an activatable element in a cellular pathway in each cell. The cells can be from solid tumor cells.

In some embodiments, the determination of the cell signaling profile of cells in different populations according to activation level of an activatable element, e.g., in a cellular pathway comprises classifying at least one of the cells as a cell that is correlated with a clinical outcome. Examples of clinical outcomes, staging, as well as patient responses are also shown above.

H. Signaling Pathways

In some embodiments, the methods described herein are employed to determine the activation level of an activatable element in a signaling pathway. In some embodiments, the cell signaling profile of a cell is determined, as described herein, according to the activation level of one or more activatable elements in one or more signaling pathways. Signaling pathways and their members have been extensively described. See Hunter T. Cell Jan. 7, 2000; 100(1): 13-27; Weinberg, 2007; and Blume-Jensen and Hunter, Nature, vol 411, 17 May 2001, p 355-365. Exemplary signaling pathways include the following pathways and their members: the JAK-STAT pathway including JAKs, and STATs: 2, 3, 4 and 5, the FLT3L signaling pathway, the MAP kinase pathway including Ras, Raf, MEK, ERK and Elk; the PI3K/Akt pathway including PI-3-kinase, PDK1, Akt and Bad; the NF-κB pathway including IKKs, IkB and NF-KB and the Wnt pathway including frizzled receptors, beta-catenin, APC and other co-factors and TCF (see Cell Signaling Technology, Inc. 2002 Catalog pages 231-279 and Hunter T., supra.). In some embodiments, the correlated activatable elements being assayed (or the signaling proteins being examined) are members of the MAP kinase, Akt, NFkB, WNT, STAT and/or PKC signaling pathways. See the description of signaling pathways in U.S. Pat. No. 8,227,202, U.S. Ser. Nos. 12/910,769. See also the pathways described in U.S. Ser. Nos. 12/703,741 and 12/687,873.

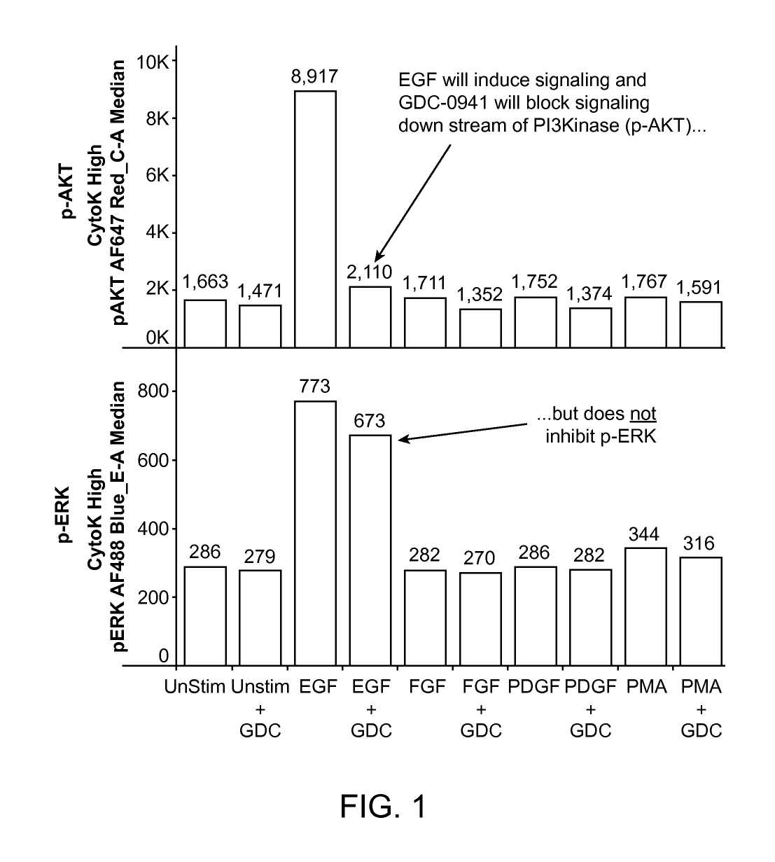

Phosphatidylinositol 3-kinases (PI3K) are ubiquitously expressed lipid kinases that phosphorylate phosphoinositides at the D-3 position of the inositol ring. The products of PI3K-catalysed reactions, phosphatidylinositol 3,4,5-trisphosphate, phosphatidylinositol 3,4 bisphosphate and phosphatidylinositol 3-phosphate are second messengers whose levels are tightly regulated by phosphatases such as Phosphatase and TENsin homologue (PTEN) that acts in an opposing role to remove the D-3 phosphate. The lipid products of PI3Ks bind or associate with pleckstrin homology containing proteins, including but not limited to the Akt serine/threonine kinases. This kinase phosphorylates a broad range of protein targets (see, for example, FIG. 1) with important consequences for a number of cellular processes, including but not limited to, proliferation, survival, metabolism, differentiation and motility. These protein target nodes are also referred to here as PI3K pathway proteins. Examples of PI3K and/or mTOR pathway proteins include, but are not limited to, p110 isoforms, PDK-1, Akt isoforms, PRAS40, Mdm2, TSC2, GSK313, BAD, FOXO transcription factors, NFkB, mTOR, p70S6 kinase, Ribosomal S6, 4EBP1, Paxillin, PKCα, PKCβ, SGK, TSC1, pBADS136, pAktS473, pPRAS40T346, pAktS308, pS6S235/236, Rictor and Raptor.

The phosphatidylinositol-3-kinase family is composed of Class 1, Class II and Class III complexes. Class I PI3Ks are heterodimeric molecules composed of a regulatory and a catalytic subunit; they are further divided between IA and IB subsets on sequence similarity. The Class IA PI3K subgroup consists of three catalytic subunits, p110α,β or δ, that form heterodimers with one of five regulatory subunits; p85α, p55α, p50α, p85β or p55γ. Class 1A PI3Ks are activated in response to many external modulators, including but not limited to, growth factors, integrins, chemokines, and cytokines. The first two p110 isoforms (α and β) are expressed in all cells, but p110δ is primarily expressed in leukocytes and it has been suggested it evolved in parallel with the adaptive immune system. The class 1B PI3K consists of one member, a heterodimer of a catalytic p110δ and a regulatory subunit which comprises p101 or p84, and is activated by G-protein coupled receptors (see Stephens, L. et al. “Phosphoinositide 3-kinases as drug targets in cancer” Curr. Opin. Pharmacology (2005) 5: 357-65).

PI3Kinases have been linked to an extraordinarily diverse group of cellular functions, including cell growth, proliferation, differentiation, motility, signal transduction, survival and intracellular trafficking Many of these functions relate to the ability of class I PI3Ks to activate Akt. The class IA PI3K p110α is mutated in many cancers (Velasco et al. Hum Pathol 37:1465-72, 2006). Many of these mutations cause the kinase to be more active. The Ptdlns(3,4,5) P3 phosphatase PTEN which antagonizes PI3K signaling is absent from many tumors. Hence, PI3K activity can contribute to cellular transformation and the development of cancer.

The PI3K pathway was first linked to cancer by the finding that the avian sarcoma virus 16 genome encodes an oncogene derived from the cellular PI3K gene. Subsequently, researchers have observed that each of the major components of the PI3K pathway is frequently mutated or overexpressed in a broad range of human cancers (Yuan, T. L. and Cantley, L. C. “PI3K pathway alteration in cancer: variations on a theme” Oncogene 27: 5497-5510, 2008). These major components of the PI3K pathway include, but are not limited to, receptor tyrosine kinases (RTKs; for example EGFR and HER2), PTEN, Akt, and the p110α subunit of PI3K. In healthy cells, ligand binding induces RTKs to activate PI3 kinase (PI3K), which phosphorylates the 3′ position of the inositol ring of phosphatidyl inositol 4-phosphate, or phosphatidyl inositol 4,5 phosphate to generate, respectively, the inositol lipid second messengers phosphatidylinositol 3,4 bisphosphate (PIP2), and phosphatidylinositol 3,4,5 bisphosphate (PIP3). These second messengers bind to the Pleckstrin Homology (PH) domains of PDK1 and Akt to recruit them to the plasma membrane, resulting in their subsequent activation through phosphorylation (for review, see Katso, R., et al., “Cellular function of phosphoinositide 3-kinases: implications for development, homeostasis, and cancer” Annu Rev Cell Dev Biol. 17: 615-75, 2001.

The p110α protein is the catalytic subunit of PI3K, and is encoded by PIK3CA. The kinase and helical domain-encoding portions of PIK3CA contain oncogenic missense mutations in up to 27% of breast, endometrial, colorectal, urinary tract, and ovarian cancers (Samuels et al., “High frequency of mutations of the PIK3CA gene in human cancers” Science 304: 554, 2004). These mutations most often occur at the hotspot codons E542, E545, and H1047, and have been shown to confer constitutive kinase activity (Samuels et al., “Mutant PIK3CA promotes cell growth and invasion of human cancer cells” Cancer Cell 7: 561-73, 2005). Furthermore, PIK3CA is frequently amplified in various human cancers (Engelman, J. A., et al. “The evolution of phosphatidylinositol 3-kinases as regulators of growth and metabolism” Nat. Rev. Genet. 7: 606-19, 2006). PTEN negatively regulates PI3K signaling in healthy cells by dephosphorylating the second messenger, PIP3. PTEN has been identified as a tumor suppressor, and mutations that inactivate PTEN are found in various cancers (Salmena, L. et al., “Tenets of PTEN Tumor Suppression” Cell 133: 403-14, 2008). Cancer cells contain PIK3CA and PTEN mutations more frequently than would be predict by chance alone, suggesting that PIK3CA gain-of-function and PTEN loss-of-function mutations are not entirely redundant in oncogenesis (Yuan, T. L. and Cantley, L. C. Oncogene 27: 5497-5510, 2008). In contrast to PIK3CA, no cancer-associated somatic mutations have been found to date in PIK3CB or PIK3CD, which encode the p110β and p110γ isoforms respectively, although overexpression of these genes in cell lines suggests that they might have tumorigenic potential. Consistent with this latter observation, increased levels of p110β and p110γ proteins have been found in a variety of cancers. Other genetic alterations within the PI3K pathway have also been also identified, including mutations in p85.

There are other clues that the different PI3K pathway components may function in different mechanisms of oncogenesis. In a mouse model of cancer generated by PTEN ablation, conditional knockout of PIK3CB, but not PIK3CA impeded tumorigenesis (Jia, S. et al., “Essential roles of PI(3)K-p110beta in cell growth, metabolism and tumorigenesis” Nature (2008) 454:776-779). Furthermore, gain-of-function mutations in PIK3CA may mediate oncogenesis through Akt-dependent and Akt-independent mechanisms (Vasudevan, K. M., et al., “Akt-independent signaling downstream of oncogenic PIK3CA mutations in human cancer” Cancer Cell (2009) 16:21-32). In some embodiments, the methods of the present invention can measure gain-of-function mutations in a PI3K pathway protein. In some embodiments, methods of the present invention can measure loss-of-function mutations in a PI3K pathway protein. In some PIK3CA-mutant cancer cell lines, P110α does not induce Akt activation, but does induce PDK1 activation and recruitment to the cell membrane. In these cell lines, PDK1 mediates SGK3 activation, which is required for survival of these cancer cells (Vasudevan, K. M., et al. Cancer Cell (2009)16:21-32). Thus, Akt signaling and SGK3 signaling may represent alternate mechanisms of PI3K-mediated oncogenesis.

Multiple studies have indicated that oncogenic alterations in PI3K signaling are not functionally equivalent, and do not necessarily just result in linear changes in signaling activity (Vasudevan, K. M., et al. Cancer Cell 16: 21-32, 2009; Yuan, T. L. and Cantley, L. C. Oncogene 27: 5497-5510, 2008). Instead, alterations in various nodes in the PI3K network may affect non-linear signaling, for example via negative feedback loops, crosstalk from other pathways, or activation of non-overlapping pathways. As an additional argument against complete functional redundancy, some mutations frequently coexist in the same tumor cell, for example PI3KCA gain-of-function and PTEN loss-of-function. There would be no co-selection for these mutations it they were functionally redundant. Instead, coexistence of two or more mutations in the pathway in the same tumor suggests selection for two or more different but synergistic mechanisms, neither of which is alone sufficient to confer oncogenecity. On the other hand, RAS mutations appear to be mutually exclusive with PIK3CA, suggesting the combination of both signaling mechanisms may be disadvantageous for cancer cells (Yuan, T. L. and Cantley, L. C. Oncogene, (2008) 27: pp. 5497-5510). Thus, the genetic background of a tumor may have important effects on the mechanism of PI3K activation. In some embodiments, the methods of the present invention include methods of measuring more than one PI3K and/or mTOR pathway protein simultaneously. The mutations can distinguish between different conditions and determine different methods of treatment to pursue.

The genetic evidence that different mechanisms of altered PI3K signaling can lead to cancer has implications for small molecule drug development. For example, the selection of which p110 isoform or isoforms to target depends on the mechanism of PI3K pathway disruption, if any, in cancer cells. Therapeutic efficacy may be improved by the selection of an appropriate targeted therapeutic or combination of therapeutics appropriate for a specific genetic background.

The p110α, β, δ and γ isoforms regulate different aspects of immune disorders. Immune disorders include inflammatory diseases, autoimmune diseases, organ and bone marrow transplant rejection and other disorders associated with T cell-mediated immune response or mast cell-mediated immune response. Non-limiting examples of immune disorders include acute or chronic inflammation, an allergy, contact dermatitis, psoriasis, rheumatoid arthritis, multiple sclerosis, type 1 diabetes, inflammatory bowel disease, Guillain-Barre syndrome, Crohn's disease, ulcerative colitis, cancer, graft versus host disease (and other forms of organ or bone marrow transplant rejection), autoimmune hemolytic anemia, autoimmune hepatitis, Berger's disease or IgA nephropathy, Celiac Sprue, chronic fatigue syndrome, dermatomyositis, fibromyalgia, Grave's disease, Hashimoto's thyroiditis, idiopathic thrombocytopenia purpura, lichen planus, multiple sclerosis, myasthenia gravis, rheumatic fever, scleroderma, Sjorgren syndrome, systemic lupus erythematosus, and vitiligo.

PI3Ks are also a key component of the insulin signaling pathway. Thus, PI3K signaling can be involved in diabetes, such as in Diabetes mellitus. The p110 associated lipid kinase activity has been demonstrated in insulinoma cells. PI3 kinase inhibition with reagents such as wortmannin and LY294002 enhances glucose-dependent insulin secretion. Activity of p110 has been demonstrated in insulinoma cells, while protein expression has been shown in human, dog, rat and mouse pancreas by immunohistochemistry.

Classic activation of the RAS/Raf/MAPK cascade occurs following ligand binding to a receptor tyrosine kinase at the cell surface, but a vast array of other receptors have the ability to activate the cascade as well, such as integrins, serpentine receptors, heterotrimeric G-proteins, and cytokine receptors. Although conceptually linear, considerable cross talk occurs between the Ras/Raf/MAPK/Erk kinase (MEK)/Erk MAPK pathway and other MAPK pathways as well as many other signaling cascades. The pivotal role of the Ras/Raf/MEK/Erk MAPK pathway in multiple cellular functions underlies the importance of the cascade in oncogenesis and growth of transformed cells. As such, the MAPK pathway has been a focus of intense investigation for therapeutic targeting. Many receptor tyrosine kinases are capable of initiating MAPK signaling. They do so after activating phosphorylation events within their cytoplasmic domains provide docking sites for src-homology 2 (SH2) domain-containing signaling molecules. Of these, adaptor proteins such as Grb2 recruit guanine nucleotide exchange factors such as SOS-1 or CDC25 to the cell membrane. The guanine nucleotide exchange factor is now capable of interacting with Ras proteins at the cell membrane to promote a conformational change and the exchange of GDP for GTP bound to Ras. Multiple Ras isoforms have been described, including K-Ras, N-Ras, and H-Ras. Termination of Ras activation occurs upon hydrolysis of RasGTP to RasGDP. Ras proteins have intrinsically low GTPase activity. Thus, the GTPase activity is stimulated by GTPase-activating proteins such as NF-1 GTPase-activating protein/neurofibromin and p120 GTPase activating protein thereby preventing prolonged Ras stimulated signaling. Ras activation is the first step in activation of the MAPK cascade. Following Ras activation, Raf (A-Raf, B-Raf, or Raf-1) is recruited to the cell membrane through binding to Ras and activated in a complex process involving phosphorylation and multiple cofactors that is not completely understood. Raf proteins directly activate MEK1 and MEK2 via phosphorylation of multiple serine residues. MEK1 and MEK2 are themselves tyrosine and threonine/serine dual-specificity kinases that subsequently phosphorylate threonine and tyrosine residues in Erk1 and Erk2 resulting in activation. Although MEK1/2 have no known targets besides Erk proteins, Erk has multiple targets including Elk-1, c-Ets1, c-Ets2, p90RSK1, MNK1, MNK2, and TOB. The cellular functions of Erk are diverse and include regulation of cell proliferation, survival, mitosis, and migration. McCubrey, J. “Roles of the Raf/MEK/ERK pathway in cell growth, malignant transformation and drug resistance” Biochimica et Biophysica Acta. (2007) 1773: 1263-1284; Friday and Adjei, Clinical Cancer Research (2008) 14, pp. 342-346.

In some embodiments, methods are employed to determine the activation level of a signaling protein in a signaling pathway known in the art including those described herein. Exemplary types of signaling proteins include, but are not limited to, kinases, kinase substrates (i.e., phosphorylated substrates), phosphatases, phosphatase substrates, binding proteins (such as 14-3-3), receptor ligands and receptors (cell surface receptor tyrosine kinases and nuclear receptors). Kinases and protein binding domains, for example, have been well described (see, e.g., Cell Signaling Technology, Inc., 2002 Catalogue “The Human Protein Kinases” and “Protein Interaction Domains” pp. 254-279).

Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, Erk1, Erk2, IKKs, GSK3a, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPB, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO, STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCyi, PLCy 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25A, Cdc25B, Cdc25C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKC β, PKCθ, PKCδ, CAMK, Elk, AFT, Myc, Egr-1, NFAT, ATF-2, Mdm2, p53, DNA-PK, Chk1, Chk2, ATM, ATR, β-catenin, CrkL, GSK3α, GSK3β, and FOXO.

In some embodiments, the methods described herein are employed to determine the activation level of an activatable element in a signaling pathway. See U.S. Ser. Nos. 61/048,886 and 61/048,920. Methods and compositions are provided for the determination of a cell signaling profile of a cell according to the status of an activatable element in a signaling pathway. Methods and compositions are provided for the determination of a cell signaling profile of cells in different populations of cells according to the status of an activatable element in a signaling pathway. In some embodiments, the determination of a cell signaling profile of cells in different populations of cells according to the activation level of an activatable element in a signaling pathway comprises classifying the cell populations as cells that are correlated with a clinical outcome. Examples of clinical outcome, staging, patient responses and classifications are shown above.

I. Binding Elements

In some embodiments, the activation level of an activatable element is determined. One embodiment makes this determination by contacting a cell from a cell population with a binding element that is specific for an activation state of the activatable element. The term “binding element” includes any molecule, e.g., peptide, nucleic acid, small organic molecule which is capable of detecting an activation state of an activatable element over another activation state of the activatable element. Binding elements and labels for binding elements are shown in U.S. Ser. No. 12/432,720, 12/229,476, 12/460,029, and 12/910,769.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999.

Methods described herein may be used to detect any particular activatable element in a sample that is antigenically detectable and antigenically distinguishable from other activatable element which is present in the sample. For example, activation state-specific antibodies can be used in the present methods to identify distinct signaling cascades of a subset or subpopulation of complex cell populations and the ordering of protein activation (e.g., kinase activation) in potential signaling hierarchies. Hence, in some embodiments the expression and phosphorylation of one or more polypeptides are detected and quantified using methods described herein. In some embodiments, the expression and phosphorylation of one or more polypeptides that are cellular components of a cellular pathway are detected and quantified using methods described herein. As used herein, the term “activation state-specific antibody” or “activation state antibody” or grammatical equivalents thereof, can refer to an antibody that specifically binds to a corresponding and specific antigen. The corresponding and specific antigen can be a specific form of an activatable element. The binding of the activation state-specific antibody can be indicative of a specific activation state of a specific activatable element.

In some embodiments, the binding element is an antibody. In some embodiment, the binding element is an activation state-specific antibody. In some embodiments, the binding element is a labeled or tagged nucelic acid.

The term “antibody” includes full length antibodies and antibody fragments, and can refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, and posses other variations. See U.S. Ser. Nos. 12/432,720, 12/229,476, 12/460,029, and 12/910,769 for more information about antibodies as binding elements.

Activation state specific antibodies can be used to detect kinase activity; however additional means for determining kinase activation are provided herein. For example, substrates that are specifically recognized by protein kinases and phosphorylated thereby are known. Antibodies that specifically bind to such phosphorylated substrates but do not bind to such non-phosphorylated substrates (phospho-substrate antibodies) can be used to determine the presence of activated kinase in a sample.

The antigenicity of an activated isoform of an activatable element can be distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of a moiety to an element, such as a phosphate moiety, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are rasied to the active site of the element, although as is known in the art, conformational changes in one area of an element may cause alterations in different areas of the element as well.

Many antibodies, many of which are commercially available (for example, see Cell Signaling Technology, <<www.cellsignal.com>> or Becton Dickinson, <<www.bd.com>>) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, SOS, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pinl prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets family transcription factors, Ets-1, Ets-2, Tel, Tel2, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1,β-catenin, FOXO, STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope can be used to produce such an antibody fragment by recombinant means well known in the art.

In alternative embodiments, aromatic amino acids of protein binding elements can be replaced with other molecules. See U.S. Ser. Nos. 12/432,720, 12/229,476, 12/460,029, and 12/910,769.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55). Further, fluorophores can be attached to such antibodies for use in the methods described herein.

A variety of recognitions structures are known in the art (e.g., Cochran et al., J. Am. Chem. Soc. (2001) 123:625-32; Boer et al., Blood (2002) 100:467-73) and can be produced using methods known in the art (see e.g., Boer et al., Blood (2002) 100:467-73; Gualillo et al., Mol. Cell. Endocrinol. (2002) 190:83-9), including for example combinatorial chemistry methods for producing recognition structures such as polymers with affinity for a target structure on an activatable protein (See e.g., Barn et al., J. Comb. Chem. 3:534-41, 2001; Ju et al., Biotechnol. 64:232-9, 1999). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In some embodiments, the recognition structure is an anti-laminin single-chain antibody fragment (scFv) (See e.g., Sanz et al., Gene Therapy 9:1049-53, 2002; Tse et al., J. Mol. Biol. 317:85-94, 2002).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986), phosphorothioate (Mag et al., Nucleic Acids Res. 19:1437, 1991; and U.S. Pat. No. 5,644,048), phosphorodithioate (Briu et al., J. Am. Chem. Soc. 111:2321, 1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895, 1992; Meier et al., Chem. Int. Ed. Engl. 31:1008). Other analog nucleic acids include those with positive backbones (Denpcy et al., Proc. Natl. Acad. Sci. USA 92:6097, 1995; non-ionic backbones (U.S. Pat. Nos. 5,386,023, 5,637,684, 5,602,240, 5,216,141 and 4,469,863; Kiedrowshi et al., Angew. Chem. Intl. Ed. English 30:423, 1991; Letsinger et al., J. Am. Chem. Soc. 110:4470, 1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395, 1994; Jeffs et al., J. Biomolecular NMR 34:17, 1994; Tetrahedron Lett. 37:743, 1996) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. pp 169-176, 1995). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used.

In some embodiments the binding element is a carbohydrate. As used herein the term carbohydrate can include any compound with the general formula (CH20)n. Examples of carbohydrates are mono-, di-, tri- and oligosaccharides, as well polysaccharides such as glycogen, cellulose, and starches.

In some embodiments the binding element is a lipid. As used herein the term lipid herein can include any water insoluble organic molecule that is soluble in nonpolar organic solvents. Examples of lipids are steroids, such as cholesterol, and phospholipids such as sphingomeylin, and fatty acyls, glycerolipids, glycerophospholipids, sphingolipids, saccharolipids, and polyketides, including tri-, di- and monoglycerides and phospholipids. The lipid can be a hydrophobic molecule or amphiphilic molecule.

Examples of activatable elements, activation states and methods of determining the activation level of activatable elements are described in U.S. Pub. No. 2006/0073474 entitled “Methods and compositions for detecting the activation state of multiple proteins in single cells” and U.S. Pub. No. 2005/0112700 entitled “Methods and compositions for risk stratification” the content of which are incorporate here by reference.

J. Modulators

In some embodiments, the methods and composition utilize a modulator. A modulator can be an activator, a therapeutic compound, an inhibitor or a compound capable of impacting a cellular pathway. Modulators can also take the form of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, a sample of cells is exposed to at least 2, 3, 4, 5, 6, 7, 8, 9, or 10 or more modulators. See U.S. Ser. No. 12/432,239 and 12/910,769 which are incorporated by reference in their entireties. See also U.S. Pat. Nos. 7,695,926 and 7,381,535 and U.S. Pub. No. 2009/0269773.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%, about 0.001% to 30%, about 0.01% to 30%, about 0.1% to 30% or 1% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, mitogens, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element. A modulator can include, e.g., a psychological stressor.

In some embodiments the modulator is selected from the group consisting of growth factors, mitogens, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g., beads, plates, viral envelopes, antigen presentation molecules such as major histocompatibility complex). In some embodiments, the modulator is a physical stimuli such as heat, cold, UV radiation, and radiation. Examples of modulators include but are not limited to Growth factors, such as Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Stromal Derived Growth Factor, (SDGF), Thrombopoietin (TPO), Transforming growth factor alpha (TGF-α), Transforming growth factor beta (TGF-β), Tumour_necrosis_factor-alpha (TNF-α), Vascular endothelial growth factor (VEGF), Keratin Derived Growht Factor (KGF), Wnt Signaling Pathway, placental growth factor (P1GF), [(Foetal Bovine Somatotrophin)] (FBS), IL-1-Cofactor for IL-3 and IL-6. Activates T cells, IL-2-T-cell growth factor. Stimulates IL-1 synthesis. Activates B-cells and NK cells, IL-3-Stimulates production of all non-lymphoid cells, IL-4-Growth factor for activated B cells, resting T cells, and mast cells, IL-5-Induces differentiation of activated B cells and eosinophils, IL-6-Stimulates Ig synthesis. Growth factor for plasma cells, and IL-7-Growth factor for pre-B cells. Cell motility factors, such as peptide growth factors, (e.g., EGF, PDGF, TGF-beta), substrate-adhesion molecules (e.g., fibronectin, laminin), cell adhesion molecules (CAMs), and metalloproteinases, hepatocyte growth factor (HGF) or scatter factor (SF), autocrine motility factor (AMF), and migration-stimulating factor (MSF). Other modulators include SDF-1α, IFN-α, IFN-γ, IL-10, IL-6, IL-27, G-CSF, FLT-3L, IGF-1, M-CSF, SCF, PMA, Thapsigargin, H₂O₂, Etoposide, Mylotarg, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L. Below are descriptions of some examples of modulators.

In one embodiment, the modulator is etoposide phosphate. Etoposide phosphate (brand names: Eposin, Etopophos, Vepesid, VP-16) can inhibit enzyme topoisomerase II. Etoposide phosphate is a semisynthetic derivative of podophyllotoxin, a substance extracted from the mandrake root Podophyllum peltatum. Etoposide can possess antineoplastic properties. Etoposide can bind to and inhibit topoisomerase II and its function in ligating cleaved DNA molecules, resulting in the accumulation of single- or double-strand DNA breaks, the inhibition of DNA replication and transcription, and apoptotic cell death. Etoposide can act primarily in the G2 and S phases of the cell cycle. See the NCI Drug Dictionary at <<www.cancer.gov/Templates/drugdictionary.aspx?CdrID=39207>>

In one embodiment, the modulator is Mylotarg. Mylotarg® (gemtuzumab ozogamicin for Injection) is a chemotherapy agent composed of a recombinant humanized IgG4, kappa antibody conjugated with a cytotoxic antitumor antibiotic, calicheamicin, isolated from fermentation of a bacterium, Micromonospora echinospora subsp. calichensis. The antibody portion of Mylotarg can bind specifically to the CD33 antigen, a sialic acid-dependent adhesion protein found on the surface of leukemic blasts and immature normal cells of myelomonocytic lineage, but not on normal hematopoietic stem cells. See U.S. Pat. Nos. 7,727,968, 5,773,001, and 5,714,586.

In one embodiment, the modulator is staurosporine. Staurosporine (antibiotic AM-2282 or STS) is a natural product originally isolated in 1977 from bacterium Streptomyces staurosporeus. Staurosporine can have biological activities ranging from anti-fungal to anti-hypertensive. See e.g., Rüegg U T, Burgess G M. “Staurosporine, K-252 and UCN-01: potent but nonspecific inhibitors of protein kinases” Trends in Pharmacological Science (1989) 10 (6): 218-220. Staruosporine can be an anticancer treatment. Staurosporine can inhibit protein kinases through the prevention of ATP binding to the kinase. This inhibition can be achieved because of the higher affinity of staurosporine for the ATP-binding site on the kinase. Staurosporine is a prototypical ATP-competitive kinase inhibitor in that it can bind to many kinases with high affinity, though with little selectivity. Staurosporine can be used to induce apoptosis. One way in which staurosporine can induce apoptosis is by activating caspase-3.

In another embodiment, the modulator is AraC. Ara-C (cytosine arabinoside or cytarabine) is an antimetabolic agent with the chemical name of 1β-arabinofuranosylcytosine. Its mode of action can be due to its rapid conversion into cytosine arabinoside triphosphate, which damages DNA when the cell cycle holds in the S phase (synthesis of DNA). Rapidly dividing cells, which require DNA replication for mitosis, are therefore affected by treatment with cytosine arabinoside. Cytosine arabinoside can also inhibit both DNA and RNA polymerases and nucleotide reductase enzymes needed for DNA synthesis. Cytarabine can be used in the treatment of acute myeloid leukaemia, acute lymphocytic leukaemia (ALL) and in lymphomas where it is the backbone of induction chemotherapy.

In another embodiment, the modulator is daunorubicin. Daunorubicin or daunomycin (daunomycin cerubidine) is a chemotherapeutic of the anthracycline family that can be given as a treatment for some types of cancer. It can be used to treat specific types of leukaemia (acute myeloid leukemia and acute lymphocytic leukemia). It was initially isolated from Streptomyces peucetius. Daunorubicin can also used to treat neuroblastoma. Daunorubicin has been used with other chemotherapy agents to treat the blastic phase of chronic myelogenous leukemia. On binding to DNA, daunomycin can intercalate, with its daunosamine residue directed toward the minor groove. It has the highest preference for two adjacent G/C base pairs flanked on the 5′ side by an A/T base pair. Daunomycin effectively binds to every 3 base pairs and induces a local unwinding angle of 11°, but negligible distortion of helical conformation.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the inhibitor is an inhibitor of a cellular factor or a plurality of factors that participates in a cellular pathway (e.g., signaling cascade) in the cell. In some embodiments, the inhibitor is a phosphatase inhibitor. Examples of phosphatase inhibitors include, but are not limited to H₂O₂, siRNA, miRNA, Cantharidin, (−)-p-Bromotetramisole, Microcystin L R, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

In some embodiments, a phenotypic profile of a population of cells is determined by measuring the activation level of an activatable element when the population of cells is exposed to a plurality of modulators in separate cultures. In some embodiments, the modulators include H₂O₂, PMA, SDF1α, CD40L, IGF-1, IL-7, IL-6, IL-10, IL-27, IL-4, IL-2, IL-3, thapsigardin and/or a combination thereof. For instance a population of cells can be exposed to one or more, all or a combination of the following combination of modulators: H₂O₂; PMA; SDF1α; CD40L; IGF-1; IL-7; IL-6; IL-10; IL-27; IL-4; IL-2; IL-3; thapsigardin. In some embodiments, the phenotypic profile of the population of cells is used to classify the population as described herein.

In some embodiments, the activation level of an activatable element in a cell is determined by contacting the cell with at least 2, 3, 4, 5, 6, 7, 8, 9, or 10 modulators. In some embodiments, the activation level of an activatable element in a cell is determined by contacting the cell with at least 2, 3, 4, 5, 6, 7, 8, 9, or 10 modulators where at least one of the modulators is an inhibitor. In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, the cell signaling profile a population of cells is determined by measuring the activation level of an activatable element when the population of cells is exposed to one or more modulators. The population of cells can be divided into a plurality of samples, and the physiological status of the population can be determined by measuring the activation level of at least one activatable element in the samples after the samples have been exposed to one or more modulators. In some embodiments, the signaling profile of different populations of cells is determined by measuring the activation level of an activatable element in each population of cells when each of the populations of cells is exposed to a modulator.

II. Prognostic and Diagnostic Methods

The methods described herein are suitable for any condition for which a correlation between the cell signaling profile of a cell and the determination of a disease predisposition, diagnosis, prognosis, and/or course of treatment in samples from individuals may be ascertained. In some embodiments, the methods described herein are directed to methods for analysis, drug screening, diagnosis, prognosis, and for methods of disease treatment and prediction. In some embodiments, the methods described herein comprise methods of analyzing experimental data. In some embodiments, the cell signaling profile of a cell population comprising a genetic alteration is used, e.g., in diagnosis or prognosis of a condition, patient selection for therapy, e.g., using some of the agents identified herein, to monitor treatment, modify therapeutic regimens, and/or to further optimize the selection of therapeutic agents which may be administered as one or a combination of agents. In some embodiments, the cell population is not associated and/or is not causative of the condition. In some embodiments, the cell population is associated with the condition but it has not yet developed the condition. The cell signaling profile of a cell population can be determined by determining the activation level of at least one activatable element in response to at least one modulator in one or more cells belonging to the cell population. The cell signaling profile of a cell population can be determined by adjusting the profile based on the presence of unhealthy cells in a sample.

In one embodiment, the methods described herein can be used to prevent disease, e.g., cancer by identifying a predisposition to the disease for which a medical intervention is available. In another embodiment, an individual afflicted with a condition can be identified and treated. In another embodiment, methods are provided for assigning an individual to a risk group. In another embodiment, methods of predicting the increased risk of relapse of a condition are provided. In another embodiment, methods of predicting the risk of developing secondary complications are provided. In another embodiment, methods of choosing a therapy for an individual are provided. In another embodiment, methods of predicting the duration of response to a therapy are provided. In another embodiment, methods are provided for predicting a response to a therapy. In another embodiment, methods are provided for determining the efficacy of a therapy in an individual. In another embodiment, methods are provided for determining the prognosis for an individual.

The cell signaling profile of a cell population can serve as a prognostic indicator of the course of a condition, e.g. whether a person will develop a certain tumor or other pathologic conditions, whether the course of a neoplastic condition in an individual will be aggressive or indolent. The prognostic indicator can aid a healthcare provider, e.g., a clinician, in managing healthcare for the person and in evaluating one or more modalities of treatment that can be used. In another embodiment, the methods provided herein provide information to a healthcare provider, e.g., a physician, to aid in the clinical management of a person so that the information may be translated into action, including treatment, prognosis or prediction.

In some embodiments, the methods described herein are used to screen candidate compounds useful in the treatment of a condition or to identify new druggable targets.

In another embodiment, the cell signaling profile of a cell population can be used to confirm or refute a diagnosis of a pre-pathological or pathological condition.

In instances where an individual has a known pre-pathologic or pathologic condition, the cell signaling profile of the cell population can be used to predict the response of the individual to available treatment options. In one embodiment, an individual treated with the intent to reduce in number or ablate cells that are causative or associated with a pre-pathological or pathological condition can be monitored to assess the decrease in such cells and the state of a cellular network over time. A reduction in causative or associated cells may or may not be associated with the disappearance or lessening of disease symptoms. If the anticipated decrease in cell number and/or improvement in the state of a cellular network do not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regime can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level, thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another cell population(s) that are associated with a good prognosis. If a beneficial population of cells is observed, measures can be taken to further increase their numbers, such as the administration of growth factors. Alternatively, individuals may be monitored for the appearance or increase in cell number of another cells population(s) associated with a poor prognosis. In such a situation, renewed therapy can be considered including continuing, modifying the present therapy or initiating another type of therapy.

In one embodiment of the invention, the present method is employed on tumor or neoplastic cells. In one embodiment the cells are from solid tumors. The solid tumor may be any solid tumor amenable to sampling for direct or indirect analysis; solid tumors include but are not limited to head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma.

A. Computational Identification of Cell Populations

In some embodiments, the activation state data of a cell population is determined by contacting the cell population with one or more modulators, generating activation state data for the cell population and using computational techniques to identify one or more discrete cell populations based on the data. These techniques are implemented using computers comprising memory and hardware. In one embodiment, algorithms for generating metrics based on raw activation state data are stored in the memory of a computer and executed by a processor of a computer. These algorithms are used in conjunction with gating and binning algorithms, which are also stored and executed by a computer, to identify the discrete cell populations. See U.S. Ser. No. 13/566,991.

The data can be analyzed using various metrics. For example, the median fluorescence intensity (MFI) is computed for each activatable element from the intensity levels for the cells in the cell population gate. The MFI values are then used to compute a variety of metrics by comparing them to the various baseline or background values, e.g., the unstimulated condition, autofluorescence, and isotype control. The following metrics are examples of metrics that can be used in the methods described herein: 1) a metric that measures the difference in the log of the median fluorescence value between an unstimulated fluorochrome-antibody stained sample and a sample that has not been treated with a stimulant or stained (log (MFIUnstimulated Stained)— log (MFIGated Unstained)), 2) a metric that measures the difference in the log of the median fluorescence value between a stimulated fluorochrome-antibody stained sample and a sample that has not been treated with a stimulant or stained (log (MFIStimulated Stained)— log (MFIGated Unstained)), 3) a metric that measures the change between the stimulated fluorochrome-antibody stained sample and the unstimulated fluorochrome-antibody stained sample log (MFIStimulated Stained)— log (MFIUnstimulated Stained), also called “fold change in median fluorescence intensity”, 4) a metric that measures the percentage of cells in a Quadrant Gate of a contour plot which measures multiple populations in one or more dimension 5) a metric that measures MFI of phosphor positive population to obtain percentage positivity above the background and 6) use of multimodality and spread metrics for large sample population and for subpopulation analysis.

In a specific embodiment, the equivalent number of reference fluorophores value (ERF) is generated. The ERF is a transformed value of the median fluorescent intensity values. The ERF value is computed using a calibration line determined by fitting observations of a standardized set of 8-peak rainbow beads for all fluorescent channels to standardized values assigned by the manufacturer. The ERF values for different samples can be combined in any way to generate different activation state metric. Different metrics can include: 1) a fold value based on ERF values for samples that have been treated with a modulator (ERFm) and samples that have not been treated with a modulator (ERFu), log 2 (ERFm/ERFu); 2) a total phospho value based on ERF values for samples that have been treated with a modulator (ERFm) and samples from autofluorecsent wells (ERFa), log 2 (ERFm/ERFa); 3) a basal value based on ERF values for samples that have not been treated with a modulator (ERFu) and samples from autofluorescent wells (ERFa), log 2 (ERFu/ERFa); 4) A Mann-Whitney statistic Uu comparing the ERFm and ERFu values that has been scaled down to a unit interval (0,1) allowing inter-sample comparisons; 5) A Mann-Whitney statistic Uu comparing the ERFm and ERFu values that has been scaled down to a unit interval (0,1) allowing inter-sample comparisons; 5) a Mann-Whitney statistic Ua comparing the ERFa and ERFm values that has been scaled down to a unit interval (0,1); and 6) A Mann-Whitney statistic U75. U75 is a linear rank statistic designed to identify a shift in the upper quartile of the distribution of ERFm and ERFu values. ERF values at or below the 75th percentile of the ERFm and ERFu values are assigned a score of 0. The remaining ERFm and ERFu values are assigned values between 0 and 1 as in the Uu statistic. For activatable elements that are surface markers on cells, the following metrics may be further generated: 1) a relative protein expression metric log 2 (ERFstain)— log 2 (ERFcontrol) based on the ERF value for a stained sample (ERFstain) and the ERF value for a control sample (ERFcontrol); and 2) A Mann-Whitney statistic Ui comparing the ERFm and ERFi values that has been scaled down to a unit interval (0,1), where the ERFi values are derived from an isotype control. See U.S. Ser. No. 13/566,991 for additional information on metrics.

The activation state data for the different markers is “gated” in order to identify discrete subpopulations of cells within the data. In gating, activation state data is used to identify discrete sub-populations of cells with distinct activation levels of an activatable element. These discrete sub-populations of cells can correspond to cell types, cell sub-types, cells in a disease or other physiological state and/or a population of cells having any characteristic in common.

In some embodiments, the activation state data is displayed as a two-dimensional scatter-plot and the discrete subpopulations are “gated” or demarcated within the scatter-plot. According to the embodiment, the discrete subpopulations may be gated automatically, manually or using some combination of automatic and manual gating methods. In some embodiments, a user can create or manually adjust the demarcations or “gates” to generate new discrete sub-populations of cells. Suitable methods of gating discrete sub-populations of cells are described in U.S. Ser. No. 12/501,295, the entirety of which is incorporated by reference herein, for all purposes.

In some embodiments, the homogenous cell populations are gated according to markers that are known to segregate different cell types or cell sub-types. In a specific embodiment, a user can identify discrete cell populations based on surface markers. For example, the user could look at: “stem cell populations” by CD34⁺, CD38^{neg} or CD34⁺, CD33^{neg} expressing cells; memory CD4 T lymphocytes; e.g., CD4⁺, CD45RA⁺,CD29^{low} cells; or multiple leukemic sub-clones based on CD33, CD45, HLA-DR, CD11b and analyzing signaling in each discrete population/subpopulation. In another alternative embodiment, a user may identify discrete cell populations/subpopulations based on intracellular markers, such as transcription factors or other intracellular proteins; based on a functional assay (e.g., dye efflux assay to determine drug transporter+cells or fluorescent glucose uptake) or based on other fluorescent markers. In some embodiments, gates are used to identify the presence of specific discrete populations and/or subpopulations in existing independent data. The existing independent data can be data stored in a computer from a previous patient, or data from independent studies using different patients.

B. Gating Methods

Gating can be used to focus on healthy cells, cells of a certain lineage, type, or to analyze cell signaling. For example, in one embodiment gating is used to identify the healthy cell subpopulation. In one embodiment, cells are identified using Forward and Side Scatter, live cells are identified using Amine Aqua, leukemic blasts are identified using Side Scatter and CD45, and non-apoptotic leukemic blasts (Healthy P1) are identified by assaying for c-PARP. This embodiment focuses the analysis on healthy cells.

In another embodiment, a user will gate cells for the cell signaling component. For example, a user may analyze the signaling in subpopulations based on surface markers. For example, the user can look at: cells that have CD45, EpCAM, or cytokeratin (cells that are CD45^{neg}/cytokeratin⁺/EpCAM⁺ are epithelial cells), “stem cell populations” by CD34⁺, CD38^{neg} or CD34⁺, CD3^{neg} expressing cells; drug transporter positive cells; i.e. C-KIT (SCF Receptor, CD117) cells⁺; FLT3⁺ cells; CD44⁺ cells, CD47⁺ cells, CD123⁺ cells, or multiple leukemic subpopulations based on CD33, CD45, HLA-DR, CD11b and analyzing signaling in each subpopulation. In another alternative embodiment, a user may analyze the data based on intracellular markers, such as transcription factors or other intracellular proteins; based on a functional assay (e.g., dye negative “side population” aka drug effluxing cells, or fluorescent glucose uptake, or based on other fluorescent markers). In some embodiments, a gate is established after learning from a responsive subpopulation. That is, a gate is developed from one data set after finding a population that correlates with a clinical outcome. This gate can then be applied restrospectively or prospectively to other data sets. See U.S. Ser. No. 12/501,295 or U.S. Pat. No. 8,227,202 for examples of gating.

Both gating embodiments can be run at the same time when a user is analyzing each well/aliquot for the activatable element that relates to cell health, for example, if each well has the reagent used for detecting the activatable element related to cell health.

In some embodiments where flow cytometry is used, prior to analyzing data the populations of interest and the method for characterizing these populations are determined. For instance, there are at least two general ways of identifying populations for data analysis: (i) “Outside-in” comparison of parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed cells under certain conditions and subsequent comparison of computationally identified cell clusters with lineage specific markers. This could be considered an inside-out approach to single cell studies as it does not presume the existence of specific populations prior to classification.

Each of these techniques capitalizes on the ability of flow cytometry to deliver large amounts of multiparameter data at the single cell level. For cells associated with a condition (e.g., neoplastic), a third “meta-level” of data exists because cells associated with a condition (e.g., cancer cells) are generally treated as a single entity and classified according to historical techniques. These techniques have included organ or tissue of origin, degree of differentiation, proliferation index, metastatic spread, and genetic or metabolic data regarding the patient.

In some embodiments, methods described herein use variance mapping techniques for mapping condition signaling space. These methods represent a significant advance in the study of condition biology because they enable comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g., two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the Chi-square (X²) test. Similarly, if two variable factors within the experiment are thought to be related, the Pearson, and/or Spearman is used to measure the degree of this relationship.

Examples of analysis for activatable elements are described in U.S. Pat. Nos. 7,563,584 and 7,393,656 the contents of which are incorporated herein by reference.

C. Molecular Labeling Methods

The methods and compositions provided herein provide binding elements comprising a label or tag. A label can be a molecule that can be directly (i.e., a primary label) or indirectly (i.e., a secondary label) detected; for example a label can be visualized and/or measured or otherwise identified so that its presence or absence can be known. Binding elements and labels for binding elements are shown, e.g., in U.S. Pat. No. 8,227,202 and U.S. Ser. Nos. 12/432,720, 12/229,476, and 12/910,769.

A compound can be directly or indirectly conjugated to a label which provides a detectable signal, e.g., radioisotopes, fluorescers, enzymes, antibodies, particles such as magnetic particles, chemiluminescers, molecules that can be detected by mass spectrometry, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments, a label can be conjugated to a binding element.

In some embodiments, one or more binding elements are uniquely labeled. Using the example of two activation state specific antibodies, “uniquely labeled” can mean that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels can fall into four classes: a) isotopic labels, which can be radioactive or heavy isotopes; b) magnetic, electrical, thermal labels; c) colored, optical labels including luminescent, phosphorous and fluorescent dyes or moieties; and d) binding partners. Labels can also include enzymes (e.g., horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be “small molecule” fluors or proteinaceous fluors (e.g., green fluorescent proteins and all variants thereof).

In some embodiments, activation state-specific antibodies are labeled with quantum dots as disclosed by Chattopadhyay, P. K. et al., “Quantum dot semiconductor nanocrystals for immunophenotyping by polychromatic flow cytometry” Nat. Med. (2006) 12, 972-977. Quantum dot labels are commercially available through Invitrogen. <<http://probes.invitrogen.com/products/qdot/>>.

Quantum dot labeled antibodies can be used alone or they can be employed in conjunction with organic fluorochrome—conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8) as well as confocal microscopy and high content cell screening or any equivalent methods.

In some embodiments, activatable elements are labeled with tags suitable for Inductively Coupled Plasma Mass Spectrometer (ICP-MS) as disclosed in Tanner et al. Spectrochimica Acta Part B: Atomic Spectroscopy, (2007) March; 62(3):188-195. See <<http www. stemspec.ca/Project/History/UofT.html.>>

Detection systems based on FRET, discussed in detail below, can be used. FRET can be used in the methods described herein, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and compositions described herein can also make use of label enzymes. A label enzyme can be an enzyme that can be reacted in the presence of a label enzyme substrate that produces a detectable product. Suitable label enzymes include but are not limited to horseradish peroxidase, alkaline phosphatase and glucose oxidase. Methods for the use of such substrates are well known in the art. The presence of a label enzyme can generally be revealed through the enzyme's catalysis of a reaction with a label enzyme substrate, producing an identifiable product. Such products may be opaque, such as the product resulting from the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989).

By radioisotope is meant any radioactive molecule. Suitable radioisotopes include, but are not limited to 14C, 3H, 32P, 33P, 35S, 125I and 131I. The use of radioisotopes as labels is well known in the art.

Labels can be indirectly detected, that is, the tag is a partner of a binding pair. “Partner of a binding pair” can mean one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide (Hopp et al., BioTechnology, (1988) 6:1204-1210; the KT3 epitope peptide (Martin et al., Science, (1992) 255: 192-194; tubulin epitope peptide (Skinner et al., J. Biol. Chem., (1991) 266:15163-15166; and the T7 gene 10 protein peptide tag (Lutz-Freyermuth et al., Proc. Natl. Acad. Sci. USA, (1990) 87:6393-6397 and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) can bind to a first antibody (second moiety) that can, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair can comprise a label, as described above. It will further be appreciated that a label allows for a tag to be indirectly labeled upon the binding of a binding partner comprising a label. Attaching a label to a tag that is a partner of a binding pair, as just described, can be referred to herein as “indirect labeling”.

“Surface substrate binding molecule” or “attachment tag” and grammatical equivalents thereof can mean a molecule have binding affinity for a specific surface substrate, which substrate is generally a member of a binding pair applied, incorporated or otherwise attached to a surface. Suitable surface substrate binding molecules and their surface substrates include, but are not limited to, poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate (Field et al., Mol. Cell. Biol., 8:2159-2165 (1988); the c-myc tag and the 8F9, 3C7, 6E10, G4, B7 and 9E10 antibody substrates thereto [Evan et al., Molecular and Cellular Biology, (1985) 5:3610-3616); and the Herpes Simplex virus glycoprotein D (gD) tag and its antibody substrate (Paborsky et al., Protein Engineering, (1990) 3(6):547-553). In general, surface binding substrate molecules include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

D. Detection Methods

In practicing the methods described herein, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods described herein can be performed according to standard techniques and protocols well-established in the art.

One or more activatable elements can be detected and/or quantified by any method that detects and/or quantitates the presence of the activatable element of interest. Such methods may include flow cytometry, mass spectrometry, radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, Far Western, Northern Blot, Southern blot, whole cell staining, immunoelectronmicroscopy, nucleic acid amplification, PCR, gene array, protein array, mass spectrometry, nucleic acid sequencing, next generation sequencing, patch clamp, 2-dimensional gel electrophoresis, differential display gel electrophoresis, microsphere-based multiplex protein assays, label-free cellular assays, etc. These techniques are particularly useful for modified protein parameters. Cell readouts for proteins and other cell determinants can be obtained using fluorescent or otherwise tagged reporter molecules. Flow cytometry and mass spectrometry methods are useful for measuring intracellular parameters. See e.g., U.S. Pat. No. 7,393,656 and Shulz et al., Current Protocols in Immunology, (2007), 78:8.17.1-20.

In some embodiments, provided herein are methods for determining an activatable element's activation profile for a single cell. The methods may comprise analyzing cells by flow cytometry on the basis of the activation level of at least two activatable elements. Binding elements (e.g., activation state-specific antibodies) can be used to analyze cells on the basis of activatable element activation level, and can be detected as described herein. Alternatively, non-binding element systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorochrome to form a detection element. Detection elements do not need to have both of the above agents, but can be one unit that possesses both qualities. These and other methods are well described in U.S. Pat. Nos. 7,381,535, 7,393,656, and 8,227,202 and U.S. Ser. Nos. 10/193,462; 11/655,785; 11/655,789; 11/655,821; 11/338,957, 12/432,720, 12/229,476, and 12/910,769.

In one embodiment, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions described herein, it will be recognized that different types of fluorescent monitoring systems, e.g., cytometric measurement device systems, can be used. In some embodiments, flow cytometric systems are used or systems dedicated to high throughput screening, e.g., 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N.J., Modern Molecular Photochemistry, Menlo Park: Benjamin/Cummings Publishing Col, Inc. (1978), pp. 296-361. Commercial instruments are available through Becton Dickinson and Beckman Coulter, among others.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy, high content cell sceening or other equivalent methods known in the art. In general, flow cytometry involves the passage of individual cells through the path of a laser beam. The scattering the beam and excitation of any fluorescent molecules attached to, or found within, the cell is detected by photomultiplier tubes to create a readable output, e.g., size, granularity, or fluorescent intensity.

In general, high content cell screening, involves labeling proteins with fluorescent tags, and finally changes in cell phenotype are measured using automated image analysis. Through the use of fluorescent tags with different absorption and emission maxima, it is possible to measure several different cell components in parallel. Furthermore, the imagining is able to detect changes at a subcellular level (e.g., cytoplasm vs. nucleus vs. other organelles). Therefore a large number of data points can be collected per cell. In addition to fluorescent labeling, various label free assays have been used in high content cell screening. In some embodiments, in invention provides mearuring the activation level of an activatable element using high content cell screening.

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, can be measured. The presence and intensity of the signals corresponding to the labels on the binding element indicates the level of activation of the activatable element on that cell (Tanner et al., Spectrochimica Acta Part B: Atomic Spectroscopy, 2007 March; 62(3):188-195.).

The detecting, sorting, or isolating step of the methods described herein can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods described herein (see e.g., WO99/54494; U.S. Pub. No. 2001/0006787).

In some embodiments, a FACS cell sorter (e.g., a FACSVantage™ Cell Sorter, Becton Dickinson Immunocytometry Systems, San Jose, Calif.) is used to sort and collect cells based on their activation profile (positive cells) in the presence or absence of an increase in activation level of an activatable element in response to a modulator. Other flow cytometers that are commercially available include the LSR II and the Canto II both available from Becton Dickinson. Other flow cytometers include the Attune Acoustic Cytometer (Life Technologies, Carlsbad Calif.) and the CyTOF (DVS Sciences, Sunnyvale, Calif.). See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g., antibodies) directed against a specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™ manual, with particular reference to sections 3-11 to 3-28 and 10-1 to 10-17, which is hereby incorporated by reference in its entirety. See the patents, applications and articles referred to, and incorporated above for detection systems.

In another embodiment, positive cells can be sorted using magnetic separation of cells based on the presence of an isoform of an activatable element. In such separation techniques, cells to be positively selected are first contacted with a specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex manual which is hereby incorporated in its entirety.

In some embodiments, methods for the determination of a receptor element activation state profile for a single cell are provided. The methods comprise providing a population of cells and analyzing the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiments, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

As will be appreciated, the methods described herein also provide for the ordering of element clustering events in signal transduction. Particularly, the methods described herein allow the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

Provided herein is a method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the methods described herein allow the individual evaluation of cells to allow true differences to be identified in a significant way.

Thus, provided herein are methods of distinguishing cellular subsets within a larger cellular population. As outlined herein, these cellular subsets often exhibit altered biological characteristics (e.g., activation levels, altered response to modulators) as compared to other subsets within the population. For example, as outlined herein, the methods described herein allow the identification of subsets of cells from a population such as primary cell populations, e.g., peripheral blood mononuclear cells that exhibit altered responses (e.g., response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such a peripheral blood mononuclear cells, or naive and memory lymphocytes.

In one embodiment of the invention, the cells from the solid tumor are present as single cells. In another embodiment of the inventions, the cells are present as a solid mass from the tumor. When necessary, cells are dispersed into a single cell suspension, e.g., by enzymatic digestion with a suitable protease, e.g., collagenase, dispase, etc; and the like. An appropriate solution is used for dispersion or suspension. Such solution will generally be a balanced salt solution, e.g., normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be fixed, e.g., with 3% paraformaldehyde, and are usually permeabilized, e.g., with ice cold methanol; HEPES-buffered PBS containing 0.1% saponin, 3% BSA; covering for 2 min in acetone at −200° C.; and the like as known in the art and according to the methods described herein.

In some embodiments, one or more cells are contained in a well of a 96 well plate or other commercially available multiwell plate. In an alternate embodiment, the reaction mixture or cells are in a cytometric measurement device. Other multiwell plates useful in the methods described herein include, but are not limited to 384 well plates and 1536 well plates. Still other vessels for containing the reaction mixture or cells and useful for the methods described herein will be apparent to the skilled artisan. Methods to automate the analysis are shown in U.S. Ser. No. 12/606,869 which is hereby incorporated by reference in its entirety.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

As will be appreciated by one of skill in the art, the instant methods and compositions find use in a variety of other assay formats in addition to flow cytometry analysis. For example, DNA microarrays are commercially available through a variety of sources (Affymetrix, Santa Clara Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, the methods and compositions described herein can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g., EGF. After incubation with the modulator, cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described herein.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods described herein include the use of liquid handling components. The liquid handling systems can include robotic systems comprising any number of components. In addition, any or all of the steps outlined herein may be automated; thus, for example, the systems may be completely or partially automated. See U.S. Ser. Nos. 12/606,869 and 12/432,239.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods described herein include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices described herein. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, fixatives, permeabilizers, assay components such as label probes, etc.

Any of the steps above can be performed by a computer program product that comprises a computer executable logic that is recorded on a computer readable medium. For example, the computer program can execute some or all of the following functions: (i) exposing reference population of cells to one or more modulators, (ii) exposing reference population of cells to one or more binding elements, (iii) detecting the activation levels of one or more activatable elements, (iv) characterizing one or more cellular pathways, (v) classifying one or more cells into one or more classes based on the activation level (vi) determining cell health status of a cell, (vii) determining the percentage of viable cells in a sample; (viii) determining the percentage of healthy cells in a sample; (ix) determining a cell signaling profile; (x) adjusting a cell signaling profile based on the percentage of healthy cells in a sample; (xi) adjusting a cell signaling profile for an individual cell based on the health of the cell; (xii) excluding or including a cell or population of cells in a cell signaling analysis based on the health of the cell or population of cells; (xiii) assaying for one or more cell health markers; and/or (xiv) assaying for one or more apoptosis and/or necrosis markers. See U.S. Ser. No. 12/606,869.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts.

The program can provide a method of determining the status of an individual by accessing data that reflects the activation level of one or more activatable elements in the reference population of cells.

E. Analysis

Advances in flow cytometry have enabled the individual cell enumeration of up to thirteen simultaneous parameters (De Rosa et al., 2001) and are moving towards the study of genomic and proteomic data subsets (Krutzik and Nolan, 2003; Perez and Nolan, 2002). Likewise, advances in other techniques (e.g., microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al., Nature Chemical Biology February 2008. Methods for the analysis of multiple parameters are well known in the art. See U.S. Ser. Nos. 11/338,957, 12/910,769, 12/293,081, 12/538,643, 12/501,274 and WO/2012/024546 for more information on analysis. See U.S. Ser. No. 12/501,295 for gating analysis. Likewise, advances in mass spectrometry allow for the identification of multiple activatable elements.

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. Flow cytometry experiments ideally include a reference sample against which experimental samples are compared. Reference samples can include normal and/or cells associated with a condition (e.g., tumor cells). See also U.S. Ser. No. 12/501,295 for visualization tools.

The patients are stratified based on nodes that inform the clinical question using a variety of metrics. To stratify the patients between those patients with No Response (NR) versus a Complete Response (CR), a prioritization of the nodes can be made according to statistical significance (such as p-value from a t-test or Wilcoxon test or area under the receiver operator characteristic (ROC) curve) or their biological relevance.

F. Indications

The methods described herein can be applicable to any cancerous condition in an individual involving, indicated by, and/or arising from, in whole or in part, an altered cell signaling profile in cells. In some embodiments, the cell signaling profile of a cell is determined by measuring characteristics of at least one cellular component of a cellular pathway in cells from different populations (e.g., different cell networks). Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pp. 232-253; Weinberg, Chapter 6, The biology of Cancer, 2007; and Blume-Jensen and Hunter, Nature, vol 411, 17 May 2001, pp. 355-365); See also U.S. Ser. No. 12/910,769. A condition involving or characterized by altered cell signaling profile can be readily identified, for example, by determining the state of one or more activatable elements in cells from different populations, as taught herein.

In some embodiments, the neoplastic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma,

Other conditions can include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz, M. Curr Opin Genet Dev 1999 October, 9(5):595-603).

### III. Kits

In some embodiments, kits are provided. Kits may comprise one or more of the state-specific binding elements described herein, such as phospho-specific antibodies. A kit may also include other reagents, such as modulators, fixatives, containers, plates, buffers, therapeutic agents, instructions, and the like. A kit can be used to assay for one or more cell health markers. A kit can be used to assay for one or more markers of apoptosis and/or necrosis.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLCγ□, PLCγ 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25A, Cdc25B, Cdc25 C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKC β, PKCθ, PKCδ, CAMK, Elk, AFT, Myc, Egr-1, NFAT, ATF-2, Mdm2, p53, DNA-PK, Chk1, Chk2, ATM, ATR, B□catenin, CrkL, GSK3α, GSK3β, and FOXO. In some embodiments, the kit comprises one or more of the phospho-specific antibodies specific for the proteins selected from the group consisting of Erk1, Erk2, Syk, Zap70, Lck, Btk, BLNK, Cbl, PLCγ2, Akt, RelA, p38, S6. In some embodiments, the kit comprises one or more of the phospho-specific antibodies specific for the proteins selected from the group consisting of Akt1, Akt2, Akt3, SAPK/JNK1,2,3, p38s, Erk1/2, Syk, ZAP70, Btk, BLNK, Lck, PLCγ, PLCγ 2, STAT1, STAT 3, STAT 4, STAT 5, STAT 6, CREB, Lyn, p-S6, Cbl, NF-kB, GSK3β, CARMA/Bcl10 and Tcl-1.

One embodiment uses a kit having the following reagents: PI3kinase or MAP kinase pathway inhibitor, EGF ligand and phenotyping, DNA content, and signaling reagents. One embodiment uses a kit further includes but not limited to: Cytokeratin-FITC, EpCAM-PerCP-Cy5.5, CD45-PE-Cy7; DNA Content dye, DAPI; Apoptosis markers, including c-PARP-AF700; and Intracellular Signaling including, pERK-PE, pAKT-AF647. In addition, various types of molecular labels for example, fluorophores, labeled nucelic acids, fluorophore-conjugated or non-conjugated antibodies and mass-tags can be supplied in the kit depending on the type of detection instrument used. In some embiodments, a kit contains insert materials with instruction on how to perform the cell anaylsis method on particular body samples. In addition in some embiodment the kit will include instructions on determining which reference profiles are mostly closely related to the patient profile generated by the cell anaylsis assay.

The state-specific binding element can be conjugated to a solid support and to detectable groups directly or indirectly. The reagents can also include ancillary agents such as buffering agents and stabilizing agents, e.g., polysaccharides and the like. The kit can further include, e.g., other members of the signal-producing system of which system the detectable group is a member (e.g., enzyme substrates), agents for reducing background interference in a test, control reagents, apparatus for conducting a test, and the like. The kit can be packaged in any suitable manner, typically with all elements in a single container along with a sheet of printed instructions for carrying out the test.

The invention provides for kits can enable the detection of activatable elements by sensitive cellular assay methods, such as mass spectrometry, IHC, laser capture microdissection, high content cell screening and flow cytometry, which are suitable for the clinical detection, prognosis, and screening of cells and tissue from patients, such as cancer patients, having a disease involving altered pathway signaling.

In some embodiments the kits can be further comprise one or more therapeutic agents. In other embodiments the kit can further comprise a software package for data analysis of cell signaling profiles, which can include reference profiles for comparison with the patient profile. In some embodiments the kits software package incudle contection to a central server to conduct for data analysis and can be reterived by the clinician, and contain a report with recommendation on disease management.

In some embodiments the kits can inculde information, such as scientific literature references, package insert materials, clinical trial results, and/or summaries of these and the like, which indicate or establish the activities and/or advantages of the composition, and/or which describe dosing, administration, side effects, drug interactions, or other information useful to a health care provider. Such information can be based on the results of various studies, for example, studies using experimental animals involving in vivo models and studies based on human clinical trials.

Kits described herein can be provided, marketed and/or promoted to health care providers, including physicians, nurses, pharmacists, formulary officials, and the like. Kits can also, in some embodiments, be marketed directly to the consumer.

### IV. Theraputeic Agents

In some embodiments, a therapeutic agent is administered to a patient based on the cell analysis method. Example therapeutic agents are described below and in the references that are incorporated above. Some compounds for inhibiting the epidermal growth factor receptor axis include Cetuximab (Eribitux), Erlotnib (Tarceva), and Gefitinib (Iressa).

In some embodiments, a therapeutic agent is administered to a patient based on the cell analysis method are molecule inhibitors. In some embodiments, a therapeutic agent is administered to a patient based on the cell analysis method are molecule inhibitors of JAK/STAT signaling. Many small-molecule inhibitors of Jak2 and other kinases are actively being developed by various pharmaceutical companies. Examples of Jak2 inhibitors and other compounds currently in development, including but not limited to: AZ-01, AZ-60, AZD 1480 (AstraZeneca-Jak2 inhibitor); ON-044580 (Onconova-non-ATP-competitive Jak2 inhibitor); SGI-1252 (SuperGen-orally available Jak2 inhibitor); TG-101348/TG-101193/TG-101209 (TargeGen-dual Jak2/Flt3 inhibitors); ITF2357 (Italfarmaco); NCB-18424, INCB-28050 (Incyte); CP-690,550; CEP-701 (Cephalon); MK-0683 (Copenhagen University Hospital Herlev-HDAC inhibitor); SB-1518, SB-1578/ONX-0805 (S*Bio); XL019 (Exelixis); bevacizumab/Avastin (Myeloproliferative DRC); Dasatinib (Bristol-Myers Squibb); Cyt-387 (Cytopia-Jak2 inhibitor); WP-1066, WP-1130 (MD Anderson Cancer Center); and VX-509 (Vertex Pharmaceuticals).

In one embodiment of the method, the compound may be an antibody conjugated to a cytotoxic drug, including, but not limited to of Mylotarg. Or the compound may be selected from the group consisting of mitoxantrone, etoposide, daunorubicin, Gleevec, Iressa, AraC, staurosporine, lenalidomide, azacitadine, Clofarabine, Zolinza and decitabine.

A large number of treatment approaches are under the process of development. Agents under investigation include Arsenic trioxide (apoptosis inducer), Sorafenib (tyrosine kinase inhibitor), Vorinostat and valproic acid (histone deacetylase inhibitors), tipifarnib and lonafarnib (farnesyl transferase), bevacizumab (anti-VEGF monoclonal antibody that inhibits angiogenesis), FG-2216 (hypoxia-inducible factor stabilizer), ezatiostat (glutathione S1 transferase inhibitor), clofarabine (nucleoside analog) (see ALAN F. LIST, et al. “Insights into the pathogenesis, Classification, and treatment of Myelodysplastic Syndromes”, Semin. Hematol. (2008) January; 45(1) 31-8). Pharmacologic differentiators, such as TLK199, (liposomal glutathione derivative) mediate proliferation and differentiation of myeloid precursors and production of GM-CSF. A TLK-199 trial on MDS patients showed hematologic improvement in all three hematopoietic lineages-erythrocytes, neutrophils, platelets. Toxicities were limited to infusion reactions, nausea, chills and bone pain. The thrombopoiesis-stimulating agent, IL-11, is an indirect thrombopoietic cytokine that helps to combat platelet dysfunction and thrombocytopenia in MDS. The major side effects of this drug include fever, fluid retention, peripheral edema, pleural effusions and atrial arrhythmias. Pegylated, recombinant human megakaryocyte growth and development factor (PEG-rHuMGDF) stimulates megakaryocyte and platelet production by binding to c-Mpl receptors.

In some embodiments, a therapeutic agent can be a FLT3 inhibitor (e.g., AC220, e.g., at 100 nM; Tandutinib [T] e.g., at 0.5 μM), a DNA damaging agent (e.g., AraC, e.g., at 0.5 μg/ml, 2 um)), A DNMT inhibitor (e.g., zazcitidine, e.g., at 2.5 μM or Decitabine, e.g., at 0.625 μM)), a PARP inhibitor (e.g., AZD2281, e.g., at 5 μM), a PI3K and mTor dual inhibitor (e.g., BEZ235, e.g., at 50 nM), a proteosome inhibitor (e.g., bortezomib at 10 nM or 50 nM), a PI3 Kdelta inhibitor (e.g., CAL-101, e.g., at 0.5 μM), a MEK inhibitor (e.g., AZD6244, e.g., at 1 μM), a DNA synthesis inhibitor (e.g., clofarabine, e.g., at 0.25 μM), a JAK inhibitor (e.g., CP690550(CP)), e.g., at 1 μM; CYT387 e.g., at 1 μM; INCB018424 at 1 μM)), a topoisomerase inhibitor (e.g., etoposide, e.g., at 15 μg/ml), a mTor inhibitor (e.g., Everolimus (RAD0001) e.g., at 10 nM), a PI3K inhibitor (e.g., GDC-0941 [G] e.g., at 1 μM), a BCR-ABL, cKit, or PDGR-R inhibitor (e.g., Imatinib e.g., at 1 μM), an HSP90 inhibitor (e.g., NVP-AUY922 e.g., at 50 nM), a VEGFR, PDGFR, RAF, FLT3, or cKIT inhibitor (e.g., Sorafenib, e.g., at 5 μM), a PDGF-R, VEGF-R, cKIT, FLT3, RET, or CSF-1R inhibitor (e.g., Sunitinib, e.g., at 50 nM), an alkylating agent (e.g., Temozolomide, e.g., at 2 μg/ml (10.3 μM), or an HDAC inhibitor (e.g., Vorinostat (SAHA, Zolinza, e.g., at 2.5 μM). See Table 1 for additional information on modulators and exemplary concentrations of the modulators. See Table 1 of PCT/US2011/01565.

A non-limiting list of chemotherapeutic agents, cytotoxic agents, and non-peptide small molecules include, but are not limited to: Gleevec® (Imatinib Mesylate), Velcade® (bortezomib), Casodex (bicalutamide), Iressa® (gefitinib), and Adriamycin; alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, the compounds or pharmaceutical composition of the present invention can be used in combination with commonly prescribed anti-cancer drugs such as Herceptin®, Avastin®, Cetuximab, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, and Zosuquidar; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Fluorocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; Taxol; thiosemicarbazone derivatives; telomerase inhibitors; arsenic trioxide; planomycin; sulindac sulfide; cyclopamine; purmorphamine; gamma-secretase inhibitors; CXCR4 inhibitors; HH signaling inhibitors; Bmi-1 inhibitors; Bcl-2 inhibitors; Notch-1 inhibitors; DNA checkpoint protein inhibitors; ABC transporter inhibitors; mitotic inhibitors; intercalating antibiotics; growth factor inhibitors; cell cycle modulators; enzymes; topoisomerase inhibitors; biological response modifiers; angiogenesis inhibitors; DNA repair inhibitors; and small G-protein inhibitors. Combinations can be made with one or more than one of the above. See U.S. Ser. No. 12/703,741.

### EXAMPLES

One embodiment of the methods described herein is applied to single cell network profiling which is referenced above. Generally, the process involves treating or inducing cells with modulator, a staining step, and a flow cytometry step. The treatment with a modulator step can start with previously frozen cells and end with cells fixed and permeabilized with a compound, such as methanol. Then the cells can be stained with an antibody directed to a particular activated protein of interest and then analyzed using a flow cytometer. These general steps are disclosed in some references referred to above, including U.S. Pat. No. 8,227,202 and U.S. Ser. No. 61/350,864.

### Example 1

### General Methology for Cell Analysis

Cell thawing, ficoll density gradient separation, and live/dead staining: Cells are thawed in a 37° C. water bath in cryovials. Once the cells are thawed, 1 mL of pre-warmed thaw buffer (RPMI+60% FBS) is added dropwise to the cryovials and then the entire contents of the cryovials are transferred to a 15 mL conical tube. The volume of each sample is brought up to 10 mL by adding the appropriate volume of thaw buffer. The 15 mL tubes are then capped and inverted 3 times.

A ficoll density gradient separation is then performed by underlaying 5 mL of ambient temperature ficoll using a Pasteur pipette on the samples. Next, the tubes are centrifuged at 400×g for 30 minutes at room temperature, the “buffy coat” aspirated, and the mononuclear cell layer transferred to a new 15 mL conical tube containing 9 mL thaw buffer. The cell layers are centrifuged at 400×g for 5 minutes, the liquid aspirated, the cell pellet gently resuspended. Subsequently, 10 mLs ambient temperature RPMI+1% FBS is added to the cell pellets and the cells centrifuged at 400×g for 5 minutes. The cell pellet is resuspended in 1 mL PBS and, if necessary, cell clumps removed by filtering (Celltrics filters) or by pipetting.

1 mL of PBS/Amine Aqua solution is added to the samples, the samples are mixed thoroughly by pipetting, and are incubated in a 37° C. water bath for 15 minutes.

After 15 minute incubation, 1 mL RPMI+10% FBS is added to the samples, a 150 μL aliquot removed from each sample and is placed in a 12×75 mm FACSTube. A cell count is performed on the AcT10 hematology analyzer. 5 mL RPMI+10% FBS are added to the samples, the cells are centrifuged at 400×g for 5 minutes, the liquid is aspirated, and the cells are resuspended at 1.25×10⁶ cells/mL in RPMI+10% FCS. The cells are kept in a 37° C. water bath until ready to array in deep-well plates.

Treatment of Cells with Modulators: A concentration for each modulator (e.g., stimulant) that is five fold (5×) more than the final concentration is prepared using Media A as diluents. The 5× modulators (e.g., stimulants) are arrayed in a standard 96 well v-bottom plate that corresponds to the well on the plate with the cells to be stimulated. Fixative is prepared by dilution of stock 16% paraformaldehyde with PBS to a concentration that is 2.4%, then placed in a 37° C. water bath. Once the plated cells have completed their incubation, the plate(s) are taken out of the incubator and placed in a 37° C. water bath next to the pipette apparatus. Prior to addition of stimulant, each plate of cells is taken from the water bath and gently swirled to resuspend any settled cells. The stimulant is pipetted into the cell plate, which is then held over a vortexer set to level “7” and mixed for 5 seconds, and followed by the return of the deep well plate to the water bath. Modulation times can include 5, 10, and 15 minutes in a 37° C. water bath. For longer incubation times, or for assays measuring induced apoptosis, cells are modulated for 6-72 hrs and restained with Amine Aqua viability dye prior to the fixation steps below.

Fixing Cells and Cell Permeabilzation: Fixation is performed using approximately 2.4% paraformaldehyde (Electron Microscopy Sciences, Hatfield, Pa.) diluted in PBS and is added to cells for a final concentration of 1.6%. The cells are pipetted up and down three times to mix and incubated for 10 minutes at 37° C. Next, the plates are centrifuged at 1000×g for 5 minutes at room temperature, the liquid aspirated from the cell pellets, and cell pellets are resuspended and the cells are permeabilized with 200 μL/well 100% ice cold methanol (SigmaAldrich), is added while vortexing. Cell plates are then covered with a foil seal and stored overnight at −80° C.

Surface and intracellular cell staining: Plates from −80° C. storage are centrifuged at 1000×g for 5 minutes at room temperature, the supernatant is aspirated, and the cell pellet is disrupted by vortexing for 10 seconds and a speed of “3000.” Then, the cell pellets are washed two times with 1 mL FACS Buffer (PBS 0.5% BSA, 0.05% NaN₃), and are incubated at room temperature at room temperature, centrifuged at 1000×g for 5 minutes at room temperature, supernatant aspirated, and the cell disrupted by vortexing as above.

Next, 20 μL of antibody cocktail is added to each well in the cell plate, the mixture is pipetted up and down 3 times to mix, and the cells are incubated at 25° C. for 1 hr or 4° C. overnight (16 hours). After incubation, cells are washed twice by the same procedure as above.

Subsequently, 10 μl of secondary antibody mix is added the cells, the mixture is pipetted up and down three times to mix, the plate covered, and the cells incubated at 25° C. for 30 minutes. After incubation, cells are washed twice by the same procedure as above.

Cell fixation and preparation for flow cytometry: The cells are then fixed by addition 1 mL of 1.6% PFA, the cells are covered and incubated at room temperature for 5 minutes. The cells are then centrifuged at 1000×g for 5 minutes, the supernatant is aspirated, the cell pellet is disrupted by vortexing as above, the cells are resuspended in 100 μL FACS Buffer, and are mixed by pipetting up and down 4 times. The mixed cells are transferred to a 96-well u-bottom plate and 100 μL of pre-diluted (40 μA into 1 mL of FACS Buffer) Sphero Rainbow 8-peak fluorescent beads to all wells. The plates are sealed with foil and placed at 4° C. in the dark until ready for acquisition on the flow cytometer.

### Example 2

### SCNP Differs Between Cleaved-PARP⁺ and Cleaved-PARR^{neg} Cell Populations

Intracellular network responses of AML patient samples were modulated by SCF and analyzed using flow-cytometry based Single Cell Network Profiling (SCNP). The protocol was similar to the protocols listed above and in U.S. Ser. Nos. 61/350,864 and 12/460,029. For each sample, the CD34⁺ cell population was further gated into cleaved-PARP⁺and cleaved-PARP^{neg} cell populations using software such as Flowjo, DIVA (available from Becton Dickinson), and/or Winlist (available from Verity Software). The Mean Fluorescence Intensity (MFI) of various signaling nodes (pAKT, pS6, and pERK) obtained by flow-cytometry based SCNP was examined in as function of cleaved-PARP gating.

Gating scheme 1 does not incorporate c-PARP as part of the analysis. Gating scheme 2 excludes all c-PARP⁺ (apoptotic) cells. Gating scheme 3 incorporates only c-PARP⁺ cells. The utility of incorporating c-PARP in the gating analysis allows one to measure signaling changes in samples or cell populations with considerable numbers of apoptotic cells.

For cell populations that appeared to have relatively few apoptotic cells, the adjusted results for the c-PARP^{neg} cell population (gating scheme 2) are similar to the results for the population which does not correct for c-PARP (gating scheme 1). However, a difference in the level of cell signaling is seen when looking at the c-PARP⁺ cells as there was very little effect from the stimulation (data not shown). Other samples showed different separation between the basal and induced state (data not shown).

For cell populations that appeared to have a relatively high number of apoptotic cells, the signaling profiles of a gated region with a majority of cleaved-PARP⁺ cells were analyzed in several donor samples. The data were also gated for cKIT⁺ in addition to cleaved-PARP⁺ or cleaved-PARP^{neg} cells. There is a difference in cell signaling when there are many apoptotic or dead cells but the cells that are c-PARP^{neg} can evoke a signal. With pAKT there is more discrimination between basal and induced signaling states in the c-PARP^{neg} population. There is no difference between the basal and induced states in c-PARP⁺ populations (gating scheme 3) or cell populations that do not adjust for c-PARP (gating scheme 1).

### Example 3

### Ananlysis of Cells in the Bladder Cancer Wash Samples

Bladder washes from non-cancer (NC) patients (pts) (n=8) and confirmed/suspected bladder cancer (BC) pts (n=20) were collected using standard medical practice and shipped overnight on ice for processing within 24 hours. Antibodies against CD45, Cytokeratin (CK), and EpCAM were used to differentiate epithelial cells from leukocytes, while measurements of DNA aneuploidy (DAPI stain) allowed for distinction between tumor and normal epithelial cells. Signaling activity in the PI3K and MAP Kinase pathways was assessed by measuring intracellular levels of p-AKT and p-ERK both at baseline and in response to pathway modulation. A process similar to that shown in Example 1 was used.

Upon delivery, cells were pelleted, counted, rested for 1 hr at 37° C., followed by a 5 minute stimulation with EGF or vehicle+/−GDC-0941 200 nM added 1 hr prior to EGF addition to inhibit PI3K signaling After cell fixation and permeabilization, samples were stained with fluorophore-conjugated antibodies/DAPI cocktail, and the data acquired using multi-parametric flow cytometry. The epithelial bladder carcinoma cell line HT-1376 was used as a control for the epithelial phenotype and EGF signaling. A DNA index was determined using the lymphoid population in the pt samples as a diploid G0/G1 reference.

66% (n=19) of the BC samples and 27% (n=4) of the NC samples met the “evaluable” criteria i.e., at least 400,000 total cells upon sample receipt and >2% of cells acquired containing an epithelial phenotype (DAPI⁺, CD45^{Low}, CK⁺, and EpCAM⁺). Measuring the DNA content with DAPI allowed us to see and gate aneuploid cells.

Of note, the majority of epithelial cells detected in the BC samples were non-apoptotic and therefore suitable for functional pathway analysis. In 3/10 BC evaluable samples a quantifiable increase in EGF-induced p-AKT and p-ERK signaling was identified (2-3 fold over baseline) and was preventable by GDC-0941 incubation, whereas no EGF-induced signaling was observed in the NC specimens.

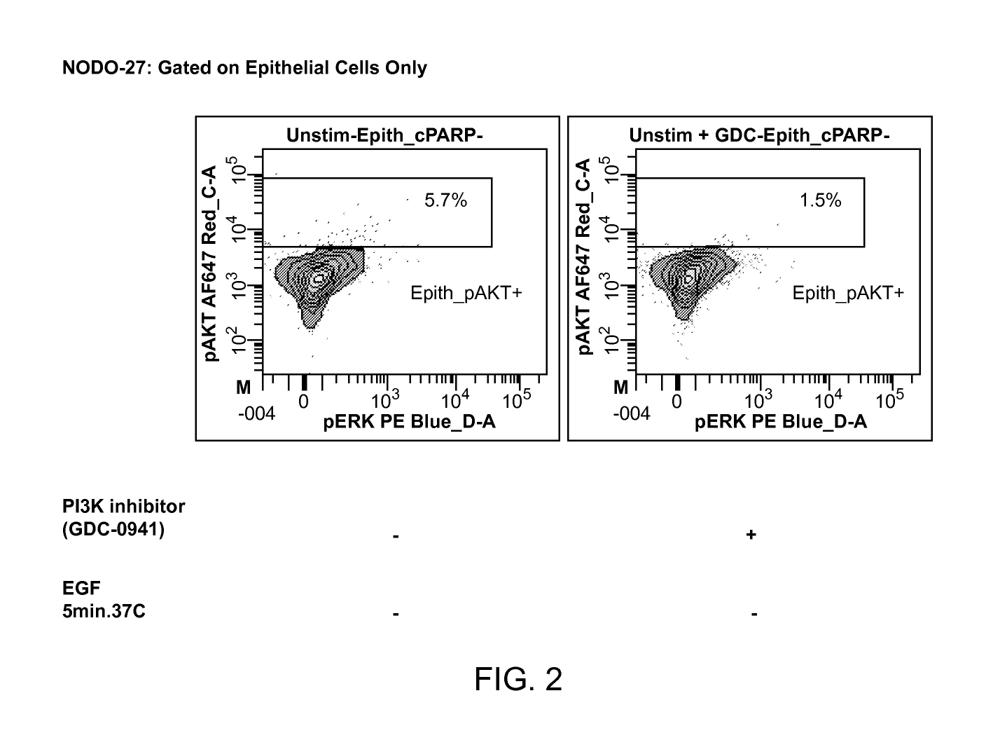

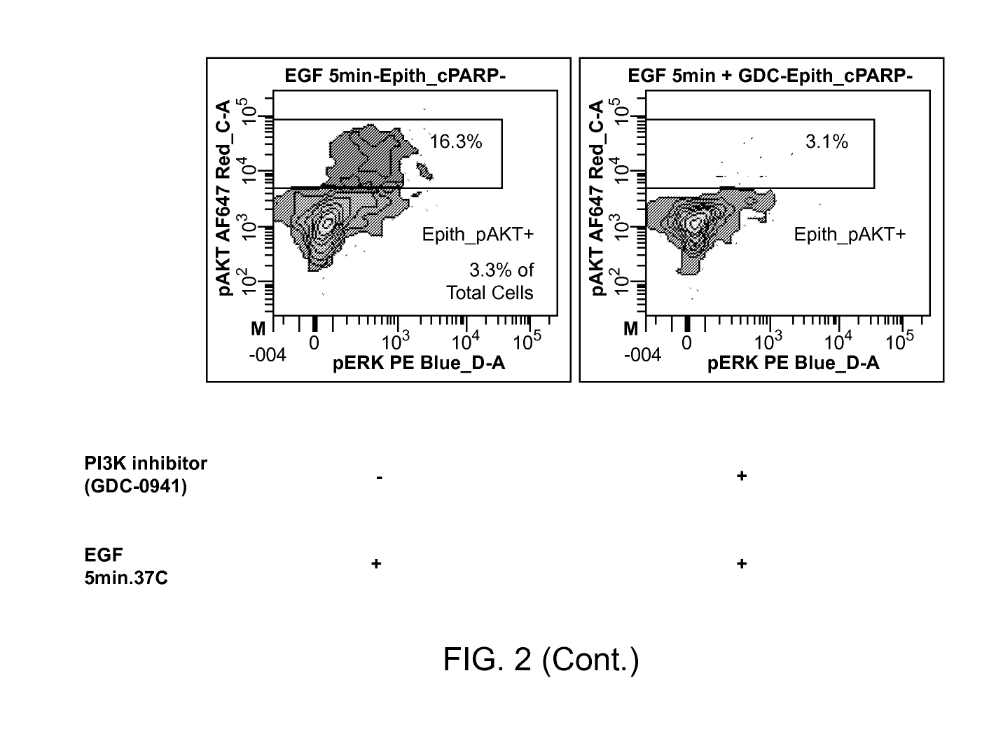

This study demonstrates the feasibility of applying SCNP, using multi-parametric flow cytometry, beyond hematological malignancies to the functional characterization of BC. Correlation of in vitro network signaling profiles with clinical outcome will provide a means to develop prognostic and predictive tools for improved BC management. See FIGS. 1 and 2 for examples of bladder cancer sample from a single patient treated in a similar manner as that shown above.

### Example 4

### Detection for Bladder Cancer Cells in Blood

A procedure similar to that shown for Example 1 was performed.

Two aliquots of 1 million cells from a bladder cancer cell line, HT-1376, were spiked into 4 mls of whole blood in an effort to determine a lower limit of detection for bladder cancer cells in blood. The epithelial cell line, HT-1376 (ATCC #CRL-1472), which has high (positive) levels of cytokeratin, low (positive) levels for EpCAM, and negative for CD45. One aliquot was preincubated with PI3K inhibitor GDC-0941 at 10 uM and another was modulated with 200 ng/mL EGF for 5 minutes at 37° C. Another aliquot of whole blood contained no HT-1376 cells or other reagents. BD phosflow fix/lyse reagent was used followed by methanol. EGF was used as a modulator to induce pAKT and pERK responses, 9.4 and 3.7 fold responses respectively. The gating scheme did not acquire any cells in the unspiked control out of 50,000 cells.

Lower numbers of HT-1376 cells were spiked into whole blood and detection limits dropped to 5 cells in 100,000 cells. Functional tests showed that the change in pAKT and pERK between stimulated and unstimulated remained relatively stable with the decreasing number of cells gated.

### Example 5

### Detection of Epithelial Cells in the Pleural Effusions

A procedure similar to that shown for Example 1 was performed. A pleural effusion was obtained from a 92 year old male patient (MCSF-04) with a diagnosis of non-small cell lung cancer with a white cell count of 21 million.

Purpose: (1) Evaluate ability to detect epithelial cells in the pleural effusion sample. (2) Measure DNA content to evaluate whether detected cells are possibly originating from the tumor (aneuploid). (3) measure basal level p-AKT and p-ERK signaling in epithelial cells. (4) Measure evoked signaling after EGF stimulation in the same cell populations. (5) Measure signaling inhibition when the EGF is added after incubation with a PI3K inhibitor (GDC-0941). (6) Culture cells in the presence and absence of EGF to observe possible growth promoting effects and to potentially characterize the signaling of cells that expand.

Cells in culture: 1 million cells were plated in two wells of a 24-well plate in the presence and absence of EGF. The cells visually appeared healthy after 48 hrs in culture with some cells with elongated appearance like an epithelial cells. Some very large round cells were also observed. At day 8, both cultures had adherent and non-adherent cells. Counts were performed. The well without EGF contained 400,000 viable, non-adherent cells while the well in the presence +EGF contained 800,000. The patient specimen contained 15.87% epithelial cells that were mostly non-apoptotic. The DNA index is 1.37 indicating the epithelial cells are aneuploid.

Gating Scheme: The epithelial phenotype is based on the cells being positive for EpCAM and Cytokeratin stains, while expressing low levels of CD45.

Results: Positive staining for c-PARP indicates apoptosis.

DNA index: The DNA index of the epithelial cells is based on the DNA content of the cells in the epithelial gate divided by the DNA content of the lymphocytes.

Epithelial cells were detected in the sample of patient MCSF-04 with 97% being negative for c-PARP staining indicating that these cells were not undergoing apoptosis and are competent for signaling.

The DNA index equaled 1.37 indicating that the epithelial cells were aneuploid and therefore tumor derived.

The high level of basal p-AKT compared to the GDC-0941 treated sample suggests that the PI3k pathway may be constitutively active. EGF induction of p-AKT and p-ERK was strong in the epithelial cells. No other cell population displayed EGF induced signaling. GDC-0941 attenuated p-AKT basal and induced signaling.

Proliferation was observed in the EGF induced condition.

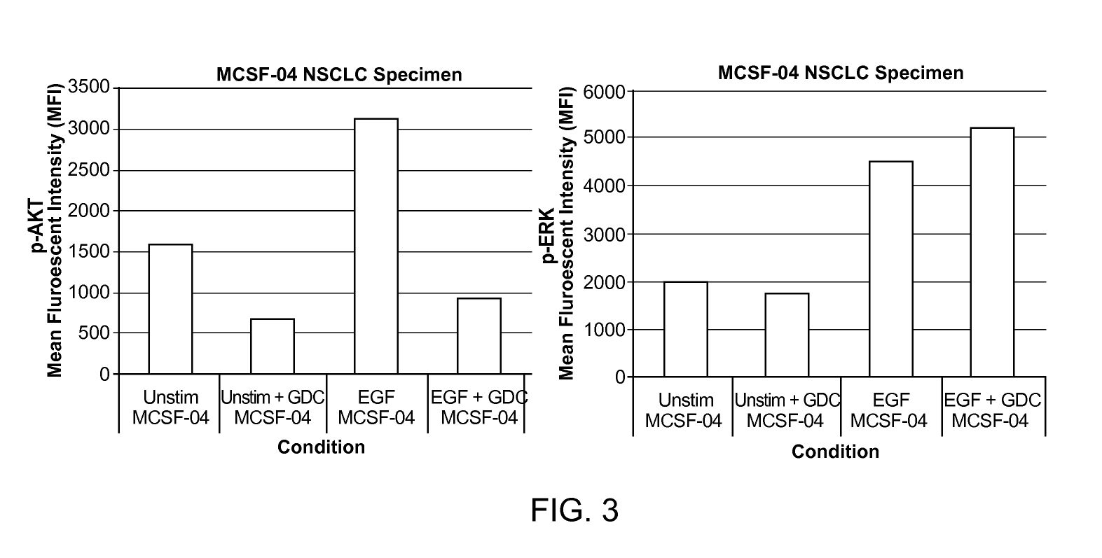

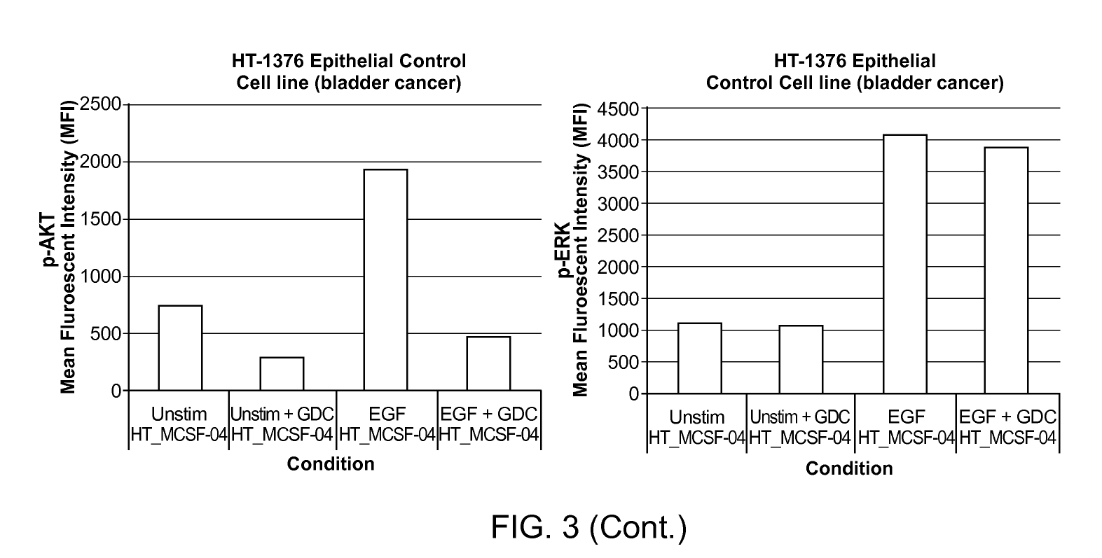

SCNP contour plots show signaling in the epithelial cell compartment only. A high level of p-AKT compared to the sample treated with PI3K inhibitor, GDC-0941, suggests that the PI3K pathway may be constitutively active. EGF stimulation (5 min., 37° C.) activates the PI3K and Ras/MEK pathways which are measured by p-AKT and p-ERK respectively. Attenuation of the EGF→p-AKT induced signaling in the presence of GDC-0941. FIG. 3 shows the median fluorescent values of the p-AKT and p-ERK levels under each treatment condition.

Overall, the data suggest that this sample contains EGF responsive cells with an epithelial phenotype, which are sensitive to inhibition with a PI3K inhibitor. Lin, et al., Cytometry A. (2010) November; 77(11):1008-19 which details the use of phosphoflow to measure IFNg induced p-STAT1 signaling in cells from pleural effusions of lung cancer patients.

### Example 6

### Detection of Epithelial Cells in Ascites Fluid

A procedure similar to that shown for Example 5 was performed. Ascites fluid was obtained from a 52 year old male patient (MCSF-02) with a diagnosis of biliary cancer with a white cell count of 4.3 million. This example has the same purpose as Example 5 as applied to ascites fluid.

Gating Scheme: The epithelial phenotype is based on the cells being positive for EpCAM and cytokeratin, while expressing low levels of CD45. Positive staining for cleaved PARP (c-PARP) indicates apoptosis. DNA index: The DNA index of the epithelial cells is based on the DNA content of the cells in the epithelial gate divided by the DNA content of the lymphocytes.

The biliary specimen contained 0.17% epithelial cells that were mostly non-apoptotic. This small percentage equates to ˜10 cells/condition. From this small population EGF induced p-AKT signaling was observed. A 9-fold increase in p-AKT levels were observed after 10 mins. EGF stimulation. Induced signaling was attenuated with a 1 hour pre-incubation with 200 nM GDC-0941 (“GDC”). There appears to be little constitutively active p-AKT due the inability of the PI3K inhibitor to decrease p-AKT beyond the basal (“unstim”) p-AKT level. This is in contrast to the control cell line. See FIG. 4.

Phosphorylated-ERK in the unstimulated condition (basal) is higher than the GDC containing sample which suggests that there is some degree of “cross-talk” between the PI3K/AKT and the MEK/ERK pathways. The cell line control has what appears to be a lower basal p-ERK level, however one must be careful when comparing absolute fluorescent levels between different cell types (ie, cell line vs primary cells) due to differences in autofluoresence and degree of non-specific antibody binding. EGF induction of p-Erk was observed in the control cell line but no significant increase above baseline was observed in the epithelial cells. The DNA index is near 1.0 indicating these cells are not aneuploid.

The vital role EGF plays in promoting angiogenesis and proliferation is thought to be primarily through activation of the RAF/MEK/ERK and PI3K/AKT/mTor pathways. Because of their efficacy in other solid tumors and the integral role of growth factors in hepatic cell carcinoma development and progression, it has been hypothesized that agents specifically targeting EGF/EGFR signaling may also be beneficial in hepatocellular carcinoma (Oishi and Wang, Int. J. Biol. Sci. 2011, 7). Recent reports suggest that the EGF receptor and HER2 pathways are suitable therapeutic targets for biliary tract carcinomas (BTCs) (Pignochino et al., BMC Cancer (2010) 10:631). BTCs express EGFR (100% for intrahepatic; 52.6% for extrahepatic), and mutations in the tyrosine kinase domain are observed in a minority of cases (15%: no report on whether these were activating mutations). EGFR inhibitors have been shown effective at blocking proliferation in BTC cell lines and in the control HT-1376 cell line.

Epithelial cells were detected in the ascites fluid of patient MCSF-2, at 0.17% of total cells. Of import to functional analysis, only a small percentage of the epithelial cells were undergoing apoptosis i.e., were cleaved-PARP positive (c-PARP⁺). c-PARP positive cells were excluded during gating analysis and the remaining cleaved-PARP negative (c-PARP^{neg}) epithelial cells were competent for signaling. No aneuploidy was detected.

EGF induction of p-AKT was strong in the epithelial cells, but no p-Erk induction above baseline was detected. No EGF induced signaling was seen in the other cell subsets analyzed.

No proliferation was observed in either condition; however the cells appear to be healthy by visual inspection.

### Example 7

### Detection of Epithelial Cells in Pleural Effusions

A procedure similar to that shown for Example 5 was performed. A pleural effusion was obtained from a 69 year old male patient (MCSF-06) with a diagnosis of non-small cell lung cancer with a white cell count of 6.8 million. This example has the same Purpose as Example 5 as applied to lung cancer. The HT-1376 epithelial bladder cancer cell line served as a positive control for EGF induced signaling.

SCNP contour plots identify p-Akt and p-ERK signaling in the epithelial cell compartment only. Basal level p-Akt is higher compared to the sample treated with GDC-0941, suggesting that the PI3K pathway may be constitutively active. EGF stimulation (5 min., 37° C.) activates the PI3K and Ras/MEK pathways which are measured by p-Akt and p-ERK respectively. Attenuation of the EGF→p-Akt but not the p-ERK signaling in the presence of GDC-0941. Median fluorescent values of p-Akt and p-ERK under each treatment condition (see FIG. 5). The control cell line was processed in parallel with the patient sample.

The patient specimen contained 0.06% epithelial cells out all nucleated cells. Epithelial cells were detected in the sample of patient MCSF-06 with 79% being negative for cleaved PARP staining, indicating that these cells were not undergoing apoptosis and are competent for signaling. DNA analysis indicated that a second epithelial G1 peak may be present with a DNA index of 1.51 indicating that the epithelial cells were aneuploid and therefore suggests the cells are tumor derived.

The elevated basal p-Akt compared to the GDC-0941 treated sample suggests that the PI3K pathway may be constitutively active. EGF induction, above basal signaling, of p-Akt and p-ERK was strong in the epithelial cells. As expected, The PI3K inhibitor GDC-0941 attenuated the basal and EGF-induced p-Akt signaling but not the p-ERK signaling. No other cell population defined by our gating hierarchy displayed EGF induced signaling.

Overall, these data suggest that this sample contains EGF responsive cells with an epithelial phenotype that are sensitive to inhibition with a PI3K inhibitor.

### Example 8

### Detection of Epithelial Cells Pleural Effusions

A procedure similar to that shown for Example 5 was performed. A pleural effusion was obtained from a 76 year old male patient (MCSF-015) with a diagnosis of non-small cell lung cancer with a white cell count of 150 million.

The HT-1376 epithelial bladder cancer cell line served as a positive control for EGF induced signaling. The control cell line was processed in parallel with the patient sample.

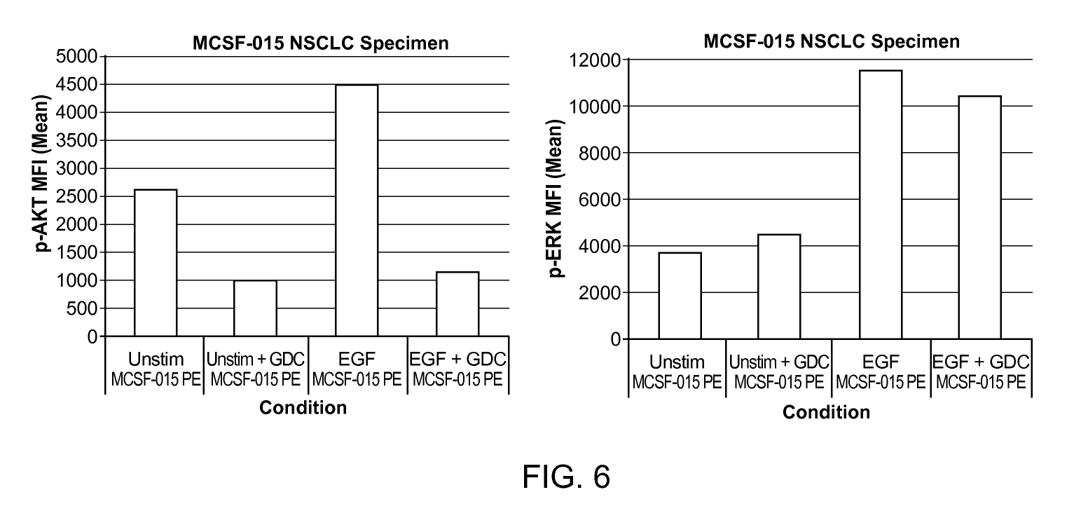

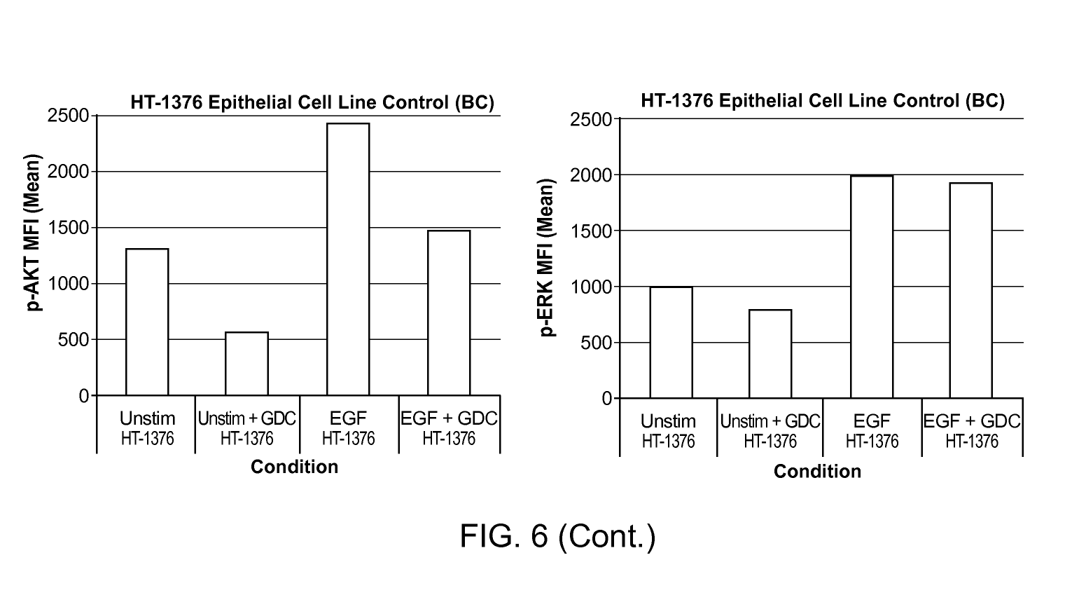

SCNP contour plots identify p-AKT and p-ERK signaling in the epithelial cell compartment only. Basal level p-Akt is higher compared to the sample treated with GDC-0941, suggesting that the PI3K pathway may be constitutively active. EGF stimulation (5 min., 37° C.) activates the PI3K and Ras/MEK pathways which are measured by p-Akt and p-ERK respectively. Attenuation of the EGF→p-AKT, but not the p-ERK signaling in the presence of GDC-0941. Median fluorescent values of p-AKT and p-ERK under each treatment condition. See FIG. 6.

The patient specimen contained 0.45% epithelial cells out all nucleated cells. Epithelial cells were detected in the sample of patient MCSF-015 with 94.5% being negative for cleaved PARP staining, indicating that these cells were not undergoing apoptosis and are competent for signaling. DNA analysis indicated that a second epithelial G1 peak may be present with a DNA index of 1.95 indicating that the epithelial cells were aneuploid and therefore suggests the cells are tumor derived.

The elevated basal p-Akt compared to the GDC-0941 treated sample suggests that the PI3K pathway may be constitutively active. EGF induction, above basal signaling, of p-Akt and p-ERK was strong in the epithelial cells. As expected, The PI3K inhibitor GDC-0941 attenuated the basal and EGF-induced p-Akt signaling but not the p-ERK signaling. No other cell population defined by our gating hierarchy displayed EGF induced signaling. Overall, these data suggest that this sample contains EGF responsive cells with an epithelial phenotype that are sensitive to inhibition with a PI3K inhibitor.

This analysis identified epithelial cells with inducible EGF signaling in ⅘ ascites or pleural effusion samples. In each of the 4 positive cases, induced signaling was inhibited by the PI3K inhibitor, GDC-0941.

### Example 9

### Isolation of CTCS in Peripheral Blood

Outline of the General method for isolation of CTCs in peripheral blood of prostate cancer patients

Cell enrichment by RosetteSep (StemCell Technologies) immunodepletion: Peripheral blood is collected in standard vacutainer blood collection tubes and RosetteSep® Human Circulating Epithelial Tumor Cell Enrichment Cocktail is added at 50 μL/mL of whole blood and incubated for 20 min at room temperature. The samples are then diluted with an equal volume of PBS+2% FBS and layered over the appropriate amount of ficoll. Next, the sample is centrifuged at 1200×g for 20 min at ambient temperature with the brake off and the plasma interface (buffy coat enriched for epithelial cells) is subsequently transferred to a new 15 mL conical tube containing 10 mL of RPMI+10% FBS and centrifuged 5 min at 400×g. The sample is then aspirated, cells resuspended in 2 mL of RPMI+10% FBS and a 30 μL aliquot is removed to determine cell count. An additional 8 mL of RPMI+10% FBS is added to the sample and then centrifuged 5 min at 400×g. The specimen is then resuspended in the appropriate volume of RPMI+10% FBS, rested for 1 hr at 37° C., followed by a 5 minute stimulation with EGF or vehicle+/−GDC-0941 (a PI3kinase inhibitor) or AZD-6244 (a MEK inhibitor) added 1 hour prior to EGF addition to inhibit PI3K or MEK signaling. After cell fixation and permeabilization, samples will be stained with fluorophore-conjugated antibodies and the data acquired using multi-parametric flow cytometry. The prostate cancer cell lines LnCAP or PC3 will be used as a control for the epithelial phenotype and EGF signaling. Sample staining will consist of antibodies against CD45, cytokeratin, EpCAM, Vimentin, N-Cadherin, and EGFR to differentiate epithelial cells (including cells undergoing epithelial to mesenchymal transition) from leukocytes. Signaling activity in the PI3K and MAPK pathways will be assessed by measuring intracellular levels of p-AKT and p-ERK both at baseline and in response to pathway modulation. A process similar to that shown in Example 1 will be used.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process for determining the signaling capability of bladder cancer cells, comprising:
Providing cells suspected of being bladder cancer cells, from an individual;
Identifying and selecting single cells that have an epithelial cell marker;
Determining aneuploidy of the cells;
Detecting and excluding cells that are undergoing apoptosis;
Contacting the cells with EGF;
Contacting the cells with a PI3kinase or MAP kinase pathway inhibitor; and
Determining the level of activatable elements in the single cells, comprising p-AKT and p-ERK.

2. A process in accordance with claim 1, further comprising administering a therapeutic agent to the individual based on the determination of the activatable elements of the single cells.

3. A process in accordance with claim 1, wherein the level of detection is less than 9 cells per 10,000,000.

4. A process in accordance with claim 1, wherein the step for determining if a cell is an epithelial cell uses reagents designed to detect EpCAM, Cytokeratin, CD45, estrogen receptor, HER2, CD44, vimentin, cadherin, or EGFR.

5. A process in accordance with claim 1, wherein the step for determining the aneuploidy of the cell uses a DNA cell content dye.

6. A process in accordance with claim 1, wherein the step for determining if a cell is undergoing apoptosis measures c-PARP, cleaved cytokeratin 18, cleaved caspase, cleaved caspase 3, cytochrome C, apoptosis inducing factor, MCL-1, BCL-2, BCL-XL, PUMA, NOXA, Bim, Bad, Bad, Bax, p53, c-myc proto-oncogene, APO-1/Fas/CD95, Annexin-V, 7-AAD, Amine Aqua, trypan blue, or propidium iodide.

7. A process in accordance with claim 1, further comprising analyzing the cells using a flow cytometer or mass spectrometer.

8. A process for analyzing solid tumor cells circulating in whole blood, comprising:
Obtaining whole blood and enriching for circulting tumor cells (CTCs);
Determining and selecting each CTC that is an epithelial cell;
Determining if the CTC is malignant, as measured by aneuploidy;
Determining and excluding any CTC that is undergoing apoptosis;
Contacting the CTC with a modulator;
Contacting the CTC with a compound to interrupt signaling in the PI3Kinase and MAP kinase pathways; and
Detecting activatable elements by contacting the cells with EGF and detecting p-Akt and P-Erk.

9. A process in accordance with claim 8, further comprising administering a therapeutic agent to the individual based on the determination of the activatable elements of the single cells.

10. A process in accordance with claim 8, wherein the level of detection is less than 9 cells per 10,000,000.

11. A process in accordance with claim 8, wherein the step for determining if a cell is an epithelial cell uses reagents designed to detect EpCAM, Cytokeratin, CD45, estrogen receptor, HER2, CD44, vimentin, cadherin, or EGFR.

12. A process in accordance with claim 8, wherein the step for determining the aneuploidy of the cell uses a DNA cell content dye.

13. A process in accordance with claim 8, wherein the step for determining if a cell is undergoing apoptosis measures c-PARP, cleaved cytokeratin 18, cleaved caspase, cleaved caspase 3, cytochrome C, apoptosis inducing factor, MCL-1, BCL-2, BCL-XL, PUMA, NOXA, Bim, Bad, Bad, Bax, p53, c-myc proto-oncogene, APO-1/Fas/CD95, Annexin-V, 7-AAD, Amine Aqua, trypan blue, or propidium iodide.

14. A process in accordance with claim 8, further comprising analyzing the cells using a flow cytometer or mass spectrometer.

15. A process for determining the signaling capability of biliary cancer cells, comprising:
Providing cells suspected of being biliary cancer cells, from an individual;
Identifying and selecting single cells that have an epithelial cell marker;
Determining aneuploidy of the cells;
Detecting and excluding cells that are undergoing apoptosis;
Contacting the cells with EGF;
Contacting the cells with a PI3kinase or MAP kinase pathway inhibitor; and
Determining the level of activatable elements in the single cells, comprising p-AKT and p-ERK.

16. A process in accordance with claim 1, further comprising administering a therapeutic agent to the individual based on the determination of the activatable elements of the single cells.

17. A process in accordance with claim 1, wherein the level of detection is less than 9 cells per 10,000,000.

18. A process in accordance with claim 1, wherein the step for determining if a cell is an epithelial cell uses reagents designed to detect EpCAM, Cytokeratin, CD45, estrogen receptor, HER2, CD44, vimentin, cadherin, or EGFR.

19. A process in accordance with claim 1, wherein the step for determining the aneuploidy of the cell uses a DNA cell content dye.

20. A process in accordance with claim 1, wherein the step for determining if a cell is undergoing apoptosis measures c-PARP, cleaved cytokeratin 18, cleaved caspase, cleaved caspase 3, cytochrome C, apoptosis inducing factor, MCL-1, BCL-2, BCL-XL, PUMA, NOXA, Bim, Bad, Bad, Bax, p53, c-myc proto-oncogene, APO-1/Fas/CD95, Annexin-V, 7-AAD, Amine Aqua, trypan blue, or propidium iodide.

21. A process in accordance with claim 1, further comprising analyzing the cells using a flow cytometer or mass spectrometer.

22. A process for analyzing solid tumor cells circulating in whole blood, comprising:
Obtaining whole blood containing CTCs;
Selecting individual CTCs that are epithelial cells;
Determining if the CTC is malignant;
Excluding any CTC that is undergoing apoptosis; and
Detecting activatable elements in the MAP Kinase or P13 kinase pathways by contacting the cells with EGF and detecting activatable elements in the MAP Kinase or P13 kinase pathways.

23. A kit for performing the processes according to claim 1:
(a) binding elements for detecting one or more proteins or peptides; and
(c) modulator for stimulating a cell's signaling pathway.

24. A kit for performing the processes according to claim 23:
(a) wherein the binding elements detect p-AKT and p-ERK;
(b) wherein the modulator is EGF.
